(19) United States
(12) Patent Application Publication
(10) Pub. No.: US 2026/0102615 A1
(43) Pub. Date: Apr. 16, 2026

(54) THALAMIC STIMULATION SYSTEM FOR THE TREATMENT OF MOTOR DISORDERS

(71) Applicant: University of Pittsburgh - Of the Commonwealth System of Higher Education, Pittsburgh, PA (US)

(72) Inventors: Elvira Pirondini, Pittsburgh, PA (US); Marco Capogrosso, Pittsburgh, PA (US); Jorge Gonzalez-Martinez, Pittsburgh, PA (US); Lucy Liang, Pittsburgh, PA (US); Jonathan Chu-an Ho, Pittsburgh, PA (US); Erinn Grigsby, Pittsburgh, PA (US)

(73) Assignee: University of Pittsburgh - Of the Commonwealth System of Higher Education, Pittsburgh, PA (US)

(21) Appl. No.: 18/863,626

(22) PCT Filed: May 15, 2023
(86) PCT No.: PCT/US2023/022238

§ 371 Date: Nov. 6, 2024

Related U.S. Application Data
(60) Provisional application No. 63/341,761, filed on May 13, 2022.

(51) Int. Cl. A61N 1/36

(52) U.S. Cl. CPC A61N 1/36067; A61N 1/36157; A61N 1/36171; A61N 1/36175

(57) ABSTRACT

Disclosed herein are methods for treating motor disorders characterized by a motor impairment. For example, particular methods comprise applying specific stimuli to ventral thalamic neurons with axons that project to premotor or motor cortex. Stimulation of areas in the ventral thalamus of a human subject with one or more electrodes with a motor disorder leads to improvement of at least one motor output associated with the motor disorder.

### CROSS REFERENCE TO RELATED APPLICATIONS

This application is the U.S. National Stage of International Application No. PCT/US2023/022238, filed May 15, 2023, which was published in English under PCT Article 21(2), which in turn claims the benefit of U.S. Provisional Application No. 63/341,761, filed May 13, 2022. The provisional is incorporated by reference in its entirety.

### FIELD

The present disclosure relates to the field of ameliorating symptoms of motor disorders by modulation of particular regions of the thalamus in a subject.

### BACKGROUND

People who suffer from a variety of disorders such as ischemic or hemorrhagic brain injury caused by stroke, traumatic brain injury, or neurodegenerative disorders such as ALS often experience motor paralysis and/or paresis in the limbs, as well as fine motor control and/or speech deficits. These deficits lead to a loss of independence, difficulty in performing everyday tasks of daily living, and/or an inability or difficulty in communicating with the external world. Although physiotherapy and speech therapy are often used to help rehabilitate lost functions or prevent further degeneration of these symptoms, the majority of the people do not recover to a satisfactory level from these conventional treatment approaches. Intense physical therapy remains the only routine intervention, but with limited efficacy, in particular for patients with moderate to severe paresis.

### SUMMARY

The primate (including humans and non-human primates) thalamus has a stereotactic and somatotopic organization that allows minimally invasive stimulation of specific thalamic nuclei. Provided herein are methods including the selective deep brain stimulation of one or more area(s) of the ventral thalamus to treat a motor disorder of a subject. In examples herein, stimulation of areas in the ventral thalamus of a human subject with a motor disorder leads to improvement of at least one motor output associated with the motor disorder. In some examples, the motor output associated with the motor disorder is a voluntary motor output (e.g., the voluntary arm, facial, and/or tongue movements of the subject). In some examples, the motor output improved by a method disclosed herein is associated with a motor impairment, but not a cognitive impairment, caused by the subject's motor disorder. In particular examples, a motor output associated with a motor disorder that is improved by a method disclosed herein is, for example, an increase in motor force and/or fine motor control.

Provided herein are methods that include the stimulation of neurons in the thalamus, which has axons that project to premotor or motor cortex; for example, to selectively stimulate the axons in one or more somatotopically and/or stereotactically defined motor and/or sensory area(s) of the ventral thalamus. In particular examples, the motor and/or sensory area(s) of the ventral thalamus includes the ventral anterior nucleus (VA), the ventral oral posterior nucleus (VOP), the ventral oral anterior nucleus (VOA), the ventral intermediate nucleus (VIM), the ventral caudal nucleus (VC), a lateral area of the VOP and/or VIM, a lateral area of the VOP and/or VIM associated with arm movements, and/or a medial area of the VOP associated with face movements. In particular examples, the motor and/or sensory area(s) of the ventral thalamus are identified using anterior commissure-posterior commissure (AC-PC)-based standard atlas coordinate calculation methods. In some examples, the motor and/or sensory area(s) of the ventral thalamus are defined by reference to one or more AC-PC-based stereotactic coordinates selected from: lateral (from about 5 to about 15 mm lateral to the AC/PC line); anterior/posterior (from about 2 to about 10 mm anterior to PC); and dorsal/ventral (from about +1 to about −2 mm from the AC/PC plane.

In some examples, the subject has a motor disorder that results from ischemic brain injury, hemorrhagic brain injury, traumatic brain injury, brain injury caused by stroke, a neurodegenerative disorder, Parkinson's disease, brain tumor(s), muscular dystrophy, myasthenia gravis, cerebral palsy, multiple sclerosis, amyotrophic lateral sclerosis (ALS), and/or transection of eloquent motor-related and/or speech-related gray or white matter.

In some examples, the motor disorder includes a motor impairment, such as, for example, a loss of muscle strength, a speech deficit, dysarthria, speech apraxia, discoordination of the oral/deglutition function, dysphagia, partial paralysis, paresis, loss of dexterity, reduced hand movement, reduced finger movement, uncontrallable muscle tone, essential tremor, and/or dystonia. In particular examples, the motor output may be a motor output associated with, for example, hand movement, finger movement, arm paresis, and/or hand paresis. In these and further examples, the motor output may be, for example, the amplitude of an arm movement of the subject, the fine motor control of an arm movement of the subject, or the amplitude of the subject's grip strength.

Methods according to some examples herein improve one or more voluntary motor outputs associated with a speech disorder that includes one or more speech motor impairment. In particular examples, the motor impairment includes, for example, dysarthria, speech arrest, and/or dysphagia. In particular examples herein, a motor output associated with a speech disorder that is improved by a method herein include one or more of, for example, a motor output associated with a lost speech deficit, an impaired speech deficit, dysphasia, and/or painful swallowing. In these and further examples, the motor output may be, for example, reduced amplitude and/or fine motor control of facial movement, neck movement, and/or tongue movement. In these and still further examples, the motor output may be, for example, reduced speech production, reduced syllabic articulation, reduced syllabic volume, increased delay between syllables, speech arrest, involuntary repetition of syllables, prolongation of sounds, interruptions in speech, indistinct pronunciation of syllables, mumbling of syllables, and/or reduced swallowing.

Provided herein are methods that include applying an electrical stimulus with one or more electrodes to the neurons in the thalamus, so as to improve the motor output(s) associated with the motor disorder. In some examples, the electrodes are configured to selectively apply the electrical stimulus to axons of the neurons one or more motor and/or sensory area(s) of the ventral thalamus. In particular examples, the electrical stimulus includes electrical pulses with an amplitude of less than about 10 mA, pulse widths between about 60 μs and about 2 ms, and/or a pulse frequency between about 20 Hz and about 100 Hz. In particular examples, the electrical pulses include charge-balanced pulses. In these and further examples, the electrical stimulus may be a continuous electrical stimulus, a closed-loop electrical stimulus, or a burst stimulation. In some examples, the frequency of the electrical stimulus is variable.

In some examples of methods disclosed herein, an electrical stimulus is applied approximately at the motor threshold of the subject. In further examples, the electrical stimulus is applied below the motor threshold. In these and still further examples, the electrical stimulus is applied approximately at the perceptual threshold of the subject. In further examples, the electrical stimulus is applied below the perceptual threshold.

In methods according to some examples herein, an electrical stimulus is applied by one or more electrodes controlled by a neurostimulator. In some examples, the neurostimulator is externalized with respect to the human subject with a motor disorder. In further examples, the neurostimulator is implanted in the human subject with a motor disorder. For example, in specific examples, the neurostimulator is fully implanted in the brain of the subject. In further examples, the neurostimulator is implanted in the chest of the subject. In still further examples, the neurostimulator is implanted in the belly of the subject. According to particular examples herein, the neurostimulator may be a daily assistive device (for example, to assist the subject in performing a voluntary movement affected by the subject's motor disorder), or may be a component of such a daily assistive device.

In some examples, a neurostimulator utilized in methods herein is a current or voltage-controlled stimulator. In particular examples, the neurostimulator controls the application of an electrical stimulus in a phasic manner. For example, the stimulation parameters of the neurostimulator may change over the course of a voluntary movement, such as, for example, one or more movements associated with speech. In some examples herein, the neurostimulator comprises at least one multiple contact lead that is configured to produce orientation-selective axonal stimulation.

Further provided herein are methods for treating a human subject having a motor disorder by selectively stimulating thalamic neurons having axons projecting to premotor or motor cortex, wherein the stimulus comprises means for selective deep brain stimulation of neurons in somatotopically and/or stereotactically defined thalamic nuclei. Means for selective deep brain stimulation of neurons in somatotopically and/or stereotactically defined thalamic nuclei include an electrical stimulus administered via implanted electrodes controlled by a neurostimulator. In examples of such methods, the selective stimulation improves at least one motor output associated with the motor disorder.

The foregoing and other objects, features, and advantages of the examples will become more apparent from the following detailed description, which proceeds with reference to the accompanying figures.

### BRIEF DESCRIPTION OF THE FIGURES

FIG. 1. Thalamic motor circuitry in primates. VOP projects to M1 which then projects to the primate facial muscles: 1: Zygomaticus minor, 2: Zygomaticus major, and 3: Orbicularis oris.

FIG. 2. Cortical activation in primates. An increased in cortical activation following VOP stimulation is identified only in M1.

FIGS. 3A and 3B. Primate EMG data. (FIG. 3A) EMGs for listed muscles. Activation increases as the frequency of the VOP stimulation increases. (FIG. 3B) EMGs for the listed muscles after thermo-coagulation. Stimulation parameters: IC—2 Hz, 2 mA, with a 3 ms PW. VOP—variable frequency (10-200 Hz), 1.5 mA (0.9 mA at 200 Hz), 100 us PW

FIG. 4. Antidromic activation from VOP stimulation. (FIG. 4A) Antidromic activation on the spinal cord following VOP stimulation (t=0). The antidromic activation is consistent stimulation frequency. There is a small amplitude decrease post-lesion. (FIG. 4B) The EMG activity of the lip. There are only clear evoked responses when the IC is also stimulated.

FIG. 5A-5C. Clinical grip force tasks. (FIG. 5A) Patients were asked to grasp a dynamometer to measure maximum grip force and grip maintenance. (FIG. 5B) We collected 6 max grip forces with stimulation (at 50 Hz) and 6 without stim (n=2 participants). We re-sampled through bootstrapping (n=10,000 iterations). We found the difference in the means of grip forces between stimulation and no stimulation to be signification (p<0.05). (FIG. 5C) We observed that VOP stimulation at 50 Hz prevented a drift in grip force that is seen with no stimulation.

FIGS. 6A-6C. Human speech therapy data. (FIG. 6A) DeepLabCut tracking markers. (FIG. 6B) Marker traces for different stimulations. (FIG. 6C) Average EMG response when smiling with different stimulations.

FIG. 7. Speech envelopes for the syllables in the phrase “tack-tad.” Top) The individual trial speech envelopes for the words Tack and Tad. Bottom) The individual trial speech envelopes during stimulation. The envelopes are slightly longer with stimulation. However, the amplitude is more stable at the middle of the envelope trace and the trace decrease is more consistent across trials.

FIG. 8. The frequency assessment for the syllables in the phrase “tack-tad.” Left) The frequency distribution for the word Tack. Notice that there is an increase in the stimulated (red) high frequency range. Also notice the slight shift and decrease in the low frequency range. Right) The frequency distribution for the work Tad. The low frequencies are consistent between stimulation and no stimulation. The only increase is for the higher frequencies.

FIG. 9. Speech articulation task. The subject was asked to perform three tongue twisters (i.e., dip-deck, tack-tad, and kick-tick) with (orange) and without (blue) stimulation on. We counted the number of errors performed by the subject and we could clearly observe that with stimulation on the number of errors was significantly reduced in all the three tongue twisters demonstrating the efficacy of VOP stimulation in restoring speech production.

FIG. 10A-10C. Characterization of monkey thalamocortical projections. (FIG. 10A) Top: High definition fiber tracking (HDFT) from VPL, VLL, and VAL nuclei (VPL: ventral posterolateral, VLL: ventral laterolateral, VAL: ventral anterolateral) of monkey motor thalamus to cortical regions (n=3) (S1: primary somatosensory cortex, M1: primary motor cortex, PMd: dorsal pre-motor cortex, SMA: supplementary motor area). Bottom: Normalized volume of thalamocortical projections (mean±standard error) from each nucleus to each cortical region normalized by the total volume of fibers. (FIG. 10B) Acute experimental setup. First, a cuff electrode was implanted around the motor branch of the radial nerve for stimulation. Animals were then implanted with a DBS electrode in the internal capsule (IC) and one in the VLL using the ROSA robot and intracortical arrays over S1 and M1. An intraspinal probe was implanted at the C6 spinal segment to record spinal local field potentials and EMG needle electrodes were inserted in arm, hand, and face muscles. A force transducer was placed in the animal's hand to measure grip force. Finally, a camera recorded the kinematics of the arm and hand. (FIG. 10C) Left: VLL Electrode implant location for each animal. Implant locations were localized from post-mortem MRI where the thermo-ablation lesion was visible (Cd: Caudate Nucleus, IC: Internal Capsule, Pt: Putamen). Right: Normalized volume HDFT projections from the area of stimulation to cortical regions (n=4).

FIG. 11A-11B. ROSA Setup/Implantation Steps. (FIG. 11A) Top: Rosa robot surgery setup. Bottom: Root mean squared registration error after registration using the fiducial screws (FIG. 11B) Step 1: plan the trajectories of each DBS probe in the ROSA One Brain planning software. Step 2: using the Rosa registration tool, register the position of the brain with fiducial screws in the skull. Step 3: an access hole is drilled into the skull along the trajectory of the probe. Step 4: fixation bolts are screwed into the skull along the probe trajectory. Step 5: DBS and IC electrodes are inserted into fixation bolts at target depth.

FIG. 12A-12D. DBS increases motor cortex excitability. (FIG. 12A) Implant location of M1 and S1 intracortical arrays. (FIG. 12B) Top: Example heatmap of average peak to peak amplitudes of cortical evoked potentials from VLL stimulation at 10 Hz across all channels over S1 and M1 for one animal. Center: Example stimulation triggered averages of cortical evoked potentials over S1 and M1 (n=40 traces) for one animal. Bottom: Histogram of peak to peak amplitudes across all channels for S1 and M1 (n=48 channels per array). (FIG. 12C) Top: Example baseline corrected spike count heatmaps in S1 and M1 for one animal. Bottom: Average spike counts over time across all channels in S1 and M1 array (n=48 channels per array). (FIG. 12D) Top: Example traces of antidromic potentials in M1 from IC stimulation without (yellow) and with (blue) conditioning from a burst of VLL stimulation for one animal (n=40 traces). Boxplot of peak to peak amplitude of the antidromic potentials when IC stimulation is conditioned by VLL stimulation at various delays (2, 5, 10, and 50 ms). For all panels, statistical significance was assessed with one-tail bootstrapping with Bonferroni correction: p<0.05 (*), p<0.01 (**), p<0.001(***).

FIG. 13A-13D. DBS amplifies motor outputs. (FIG. 13A) Example of Flexor Digitorum Minimi (FDM) motor evoked potentials (MEPs) generated by IC stimulation at 2 Hz paired with continuous VLL stimulation with gradual ramp up of amplitude (0 to 3 mA). (FIG. 13B) MEPs of one arm muscle (n=40, Biceps, Mk-JC) and one hand muscle (n=40, EDC, Mk-HS) with IC stim alone and then paired with VLL stim at 50 Hz and 100 Hz. Boxplots of the peak to peak amplitudes of the MEPs (Biceps, FCR: Flexor Carpi Radialis, EDC: Extensor Digitorum Communis). (FIG. 13C) Force transducer experimental setup and stimulation parameters (IC 45-50 Hz burst, is on, 2s off; VLL 50 Hz continuous). Example force traces (n=20) and boxplots of area under the curve (IC alone, IC with VLL at 50 Hz, and IC with VLL at 100 Hz). (FIG. 13D) MEPs of one face muscle (n=40, Orbicularis Oris, Mk-OP) with IC stim alone and then paired with VLL stim at 50 Hz and 100 Hz and boxplots of peak to peak amplitudes. For all panels, statistical significance was assessed with one-tail bootstrapping with Bonferroni correction: p<0.05 (*), p<0.01 (**), p<0.001(***).

FIG. 14. VLL Stimulation amplifies MEP across arm, hand, and face muscles. Boxplots of peak to peak amplitude of MEPs across multiple muscles (APB: Abductor Pollicis Brevis, FDC: Flexor Digitorum Communis, FDM: Flexor Digiti Minimi, EDC: Extensor Digitorum Communis, ECR: Extensor Carpi Radialis, BIC: Biceps, Buc: Buccinator, Mas: Masseter, O. Oris: Orbicularis Oris) in n=3 animals (MK-OP, MK-HS, MK-JC) where EMG recordings were performed. MEPs were recorded during IC stimulation alone and then paired with VLL stimulation at 50 Hz and 100 Hz. For all panels, statistical significance was assessed with one-tail bootstrapping with Bonferroni correction: p<0.05 (*), p<0.01 (**), p<0.001(***).

FIG. 15A-15B. VLL stimulation potentiates movements of the arm and hand. (FIG. 15A) Example kinematic trace from MK-SZ with IC alone and paired with VLL stimulation at 50 Hz and VLL at 100 Hz. (FIG. 15B) Scatter plots for the arm (wrist marker) and hand (thumb and index marker), representing the percentage variation of the peak to peak amplitude, in n=3 animals (MK-SZ, MK-HS, MK-JC). Kinematics were recorded during IC stimulation alone and then with paired VLL stimulation at 50 Hz and 100 Hz. For all panels, statistical significance was assessed with one-tail bootstrapping with Bonferroni correction: p<0.05 (*), p<0.01 (**), p<0.001(***).

FIG. 16A-16D. Motor output potentiation is not through spinal circuits. (FIG. 16A) Example of antidromic responses in the spinal cord from VLL stimulation at 10 Hz (n=30 traces) in one monkey. (FIG. 16B) Left: Heatmaps of peak to peak amplitude of the antidromic responses in the spinal cord at the C6-C7 spinal level with dorsal-ventral alignments for n=3 animals. The dashed boxes are highlighting the putative intermediate-ventral zone where we see the greatest responses. Right: Boxplots of the antidromic response latency for each animal (n=656 MK-OP, n=596 MK-JC, n=289 MK-HS). (FIG. 16C) MEPs of the wrist (ECR: Extensor Carpi Radialis, 30 traces for each animal) elicited by VLL stim at 10 Hz. (FIG. 16D) EMG reflexes of the ECR muscle elicited by radial nerve stimulation and radial nerve paired with continuous VLL stimulation at 50 Hz (30 example traces each). Boxplots of the peak to peak amplitudes of the EMG reflexes elicited by radial nerve stimulation alone and radial nerve stimulation paired with continuous VLL stimulation at 50 Hz. Statistical significance was assessed with one-tail bootstrapping with Bonferroni correction, however, in all cases the results were not significant.

FIG. 17A-17B. MEP responses from VLL stimulation. (FIG. 17A) Example MEPs (30 traces for each plot) of one arm, hand, wrist, and face muscle elicited by VLL stimulation at either 10 Hz (top row) or 50 Hz (bottom row). (FIG. 17B) Boxplots of the peak to peak MEP responses to VLL stimulation at 10 and 50 Hz for arm (BIC: Bicep), hand (ABP: Abductor Pollicis Brevis and APL: Aductor pollicis longus), wrist (ECR: Extensor Carpi Radialis, EDC: Extensor Digitorum Communis, FCR: Flexor Carpi Radialis, FDC: Flexor digitorum superficialis, and FDM: Flexor digiti minimi brevis), and face (O. Oris: Orbicularis oris) muscles for three animals (MK-OP, MK-JC, MK-HS).

FIG. 18. Radial nerve MEPs. Boxplots of the peak to peak amplitudes of the EMG reflexes for ECR (top row) and EDC (bottom row) elicited by radial nerve stimulation alone and radial nerve stimulation paired with continuous VLL stimulation at 50 Hz for 3 animals (MK-JC, MK-OP, MK-HS). For all panels, statistical significance was assessed with one-tail bootstrapping with Bonferroni correction, however, in all cases the results were not significant.

FIG. 19A-19D. Responses are modulated in a frequency-dependent manner. (FIG. 19A) Examples of frequency-dependent modulation of muscular responses. EMG responses were elicited by 2 Hz stimulation of the IC paired with different VLL stimulation frequencies (10, 50, 80, 100, and 200 Hz). The stimulation amplitude for both IC and VLL were held constant for all conditions. (FIG. 19B) The occurrence frequency of modulation patterns with respect to stimulation frequency. All patterns recorded in all muscles of 3 animals in which high-frequency stimulation was tested were included in the analysis (n=35 patterns at 10 Hz, n=56 patterns at 50 Hz, n=30 patterns at 80 Hz, n=90 at 100 Hz, and n=21 patterns at 200 Hz). (FIG. 19C) Top: Example of spinal responses in the ventral zone for IC stimulation alone and IC stimulation paired with VLL stimulation at 80 and 100 Hz (n=30 traces per plot). Bottom: Heatmaps of the average area under the curve (AUC) calculated from 5 to 10 ms after IC stimulation for all ventral channels for IC stimulation alone (n=77 MK-HS, n=37 MK-JC) and IC stimulation paired with VLL stimulation at 10 (n=227 MK-HS, n=113 MK-JC), 50 (n=222 MK-HS, n=90 MK-JC), 80 (n=233 MK-HS, n=90 MK-JC), and 100 Hz (n=234 MK-HS, n=90 MK-JC). (*) for significant potentiation and (+) for significant suppression. Bold represents p<0.001. (FIG. 19D) Top: Schematic of the experimental layout for testing frequency dependence within the motor cortex. Bottom: Example traces of the cortical evoked potential responses in the M1 array when stimulating the thalamus at different frequencies (10, 50, 80, and 100 Hz) (n=30 traces). Dashed lines show the average bound of the peak to peak values. Boxplots of the peak to peak amplitudes of the cortical evoked potentials. Statistical significance was tested by comparing 50 Hz VLL stimulation to all other stimulation conditions for potentiation using one-tailed bootstrapping with Bonferroni correction: p<0.05 (*), p<0.01 (**), p<0.001(***).

FIG. 20A-20D. DBS amplifies motor outputs after CST lesions. (FIG. 20A) Left: T2-weighted post-mortem MRI of IC lesion and VLL location (axial plane). (Cu: Caudate Nucleus, IC: Internal Capsule, Pt: Putamen). Center: HDFT of the corticospinal tract (CST) in intact and lesioned hemispheres. Right: volume of CST (mean +− standard error over animals) for both hemispheres normalized over the sum of the volumes in both hemispheres. (FIG. 20B) Example of post-lesion MEPs of one hand muscle (n=40, ECR: Extensor Communis Radialis, Mk-JC). Boxplots of peak to peak amplitude of MEP pre- and post-lesion for IC alone, and IC with VLL 50 Hz, VLL 80 Hz and VLL 100 Hz. (FIG. 20C) Boxplot of peak to peak amplitudes of MEPs in one arm muscle (Biceps), one hand muscle (EDC: Extensor digitorum communis), and one face muscle (Orbicularis Oris) for pre- and post-lesion for IC alone, and IC with VLL 100 Hz. (FIG. 20D) Top: Example of force traces (n=20). Bottom: boxplot of AUC pre- and post-lesion for IC alone, and IC with VLL 50 Hz, VLL 80 Hz, and VLL 100 Hz. For all panels, statistical significance was assessed with one-tail bootstrapping with Bonferroni correction: p<0.05 (*), p<0.01 (**), p<0.001(***).

FIG. 21. Potentiation of MEP persists after CST Lesion across arm, hand, and face muscles. Boxplots of peak to peak amplitudes of MEPs across multiple muscles (APB: Abductor Pollicis Brevis, FDC: Flexor Digitorum Communis, FDM: Flexor Digiti Minimi, EDC: Extensor Digitorum Communis, ECR: Extensor Carpi Radialis, BIC: Biceps, Buc: Buccinator, Mas: Masseter, O. Oris: Orbicularis Oris) in n=3 animals (MK-OP, MK-HS, MK-JC) where EMG recordings were performed after lesion of the CST. MEPs were recorded during IC stimulation alone before and after the CST lesion and then with paired VLL stimulation at 50, 80, and 100 Hz. For all panels, statistical significance was assessed with one-tail bootstrapping with Bonferroni correction: p<0.05 (*), p<0.01 (**), p<0.001(***).

FIG. 22A-22B. Potentiation of movements persists after CST Lesion. (FIG. 22A) Example kinematic trace from MK-JC with IC alone and paired with VLL at 50, 80, and 100 Hz after lesion of the CST. (FIG. 22B) Scatter plots for the arm (wrist marker) and hand (thumb, index, and pinky marker), representing the percentage variation of the peak to peak amplitude, in n=2 animals (MK-SZ and MK-JC). Traces were created in DeepLabCut with markers placed on the thumb, index finger, pinky finger, and wrist. Kinematics was recorded during IC stimulation alone and IC stimulation paired with VLL stimulation at 50, 80, and 100 Hz. For all panels, statistical significance was assessed with one-tail bootstrapping with Bonferroni correction: p<0.05 (*), p<0.01 (**), p<0.001(***).

FIG. 23A-23G. DBS amplifies motor outputs in humans. (FIG. 23A) Top: Experimental setup for human intraoperative experiments. Enlargement shows a schematic representing the subdural strip electrode placement over the primary motor (M1) and somatosensory (S1) cortices, and the phase reversal (PR) to identify the central sulcus. Needle electrodes were inserted in arm, wrist, and hand muscles to record MEPs and superficial electrodes were placed over the median nerve for SSEP. (FIG. 23B) Left: HDFT from the VIM/VOP to cortical regions. Right: Normalized volume (mean±standard error over n=4 subjects) of VIM/VOP projections to each cortical region normalized by the total volume of fibers. (FIG. 23C) Top: Example traces (n=122) of cortical evoked potentials elicited by VIM/VOP stimulation recorded over an S1 (left) and PR (right) contact for one subject (S04). Bottom: Box-plots of peak to peak amplitude of cortical evoked potentials at S1 and PR contact. From left to right, subjects 1 to 4 are shown (n=128, n=585, n=601, n=122 trials, respectively). (FIG. 23D) Example MEP traces (arm, biceps; wrist, flexor; n=60) with VIM/VOP stimulation alone for S03. (FIG. 23E) Example MEP traces with DCS alone and DCS paired with VIM/VOP stimulation at 50 and 100 Hz. Arm is S03 biceps (from left to right, n=48, n=60, n=60), hand is S01 abductor pollicis brevis (from left to right, n=20, n=16, n=20). (FIG. 23F) Box plots of AUC for MEPs of the arm and hand (biceps and abductor pollicis brevis respectively; n=58) with DCS alone and DCS paired with VIM/VOP stimulation at 50 and 100 Hz. (FIG. 23G) Scatter plots for arm, hand, wrist, and hand muscles of all subjects, representing the percentage of AUC variation, with respect to DCS alone, for all the different VIM/VOP stimulation frequencies (50, 80 and 100 Hz). For all panels, statistical significance was assessed with one-tail bootstrapping with Bonferroni correction: p<0.05 (*), p<0.01 (**), p<0.001(***).

FIG. 24A-24B. Human DBS Volume of Tissues Activation. (FIG. 23A) Left: Reconstructions of the VIM/VOP deep brain stimulation (DBS) electrode from one subject (S01). Simulated volume of tissue activation (VTA) at the cathode and anode (bipolar stimulation). Right: Aggregation of VTAs from n=4 human participants (S01, S02, S03, S04). (FIG. 23B) Left: Reconstructions of VIM/VOP DBS electrodes and VTA from TBI01. Right: modeled VTA from VIM/VOP DBS for TBI01.

FIG. 25. DBS potentiates MEPs across arm, wrist and hand muscles in humans. Box plots of MEPs AUC amplitudes of different muscles with DCS alone and DCS paired with VIM/VOP stimulation at 50, and/or 80 Hz and/or 100 Hz. All subjects (S01, S02, S03 and S04) are reported. Amplitudes refer to the current amplitudes for DCS. APB, abductor pollicis brevis, FLEX, flexors; EXT, extensors; BI, biceps; TRI, triceps. For all panels, statistical significance was assessed with one-tail bootstrapping with Bonferroni correction: p<0.05 (*), p<0.01 (**), p<0.001(***).

FIG. 26A-26F. DBS improves voluntary motor control after TBI. (FIG. 26A) MRI of TBI01 with segmented lesions in grey circles. (FIG. 26B) Box-plots of peak to peak amplitude of cortical evoked potentials over S1 and PR contact from VIM/VOP stimulation at 10 Hz (n=599). (FIG. 26C) Left: Example of MEPs with DCS alone and DCS paired with VIM/VOP stimulation at 50 (n=18) and 100 Hz (n=40) for hand (APB) and wrist (EDC). Right: box plots of MEPs AUC of ABP and EDC (n=58) with DCS alone and DCS paired with VIM/VOP stimulation at 50 (n=18) and 100 Hz (n=40). (FIG. 26D) Schema of the motor task performed by TB01. (FIG. 26E) Left: Example of force traces without (top) and with (bottom) VIM/VOP stimulation at 55 Hz. Right: bar plot of the root-mean-square error of the force without (yellow) and with (blue) VIM/VOP stimulation at 55 Hz. (FIG. 26F) Top: average power spectrums from 1 to 12 Hz calculated over the hold periods of the task without (yellow) and whit (blue) stimulation at 55 Hz. Bottom: boxplots of the average power spectrum over the clinical observed range for ET (6-12 Hz). For all panels, statistical significance was assessed with one-tail bootstrapping with Bonferroni correction: p<0.05 (*), p<0.01 (**), p<0.001(***).

FIGS. 27A-27C. DBS potentiates kinematics in the face during volitional movements. (FIG. 27A) Left: Schematic of the experimental setup, highlighting the position on the face we identified and tracked with DeepLabCut. Example kinematic traces are shown for three facial markers while the participant performs the smile-neutral-smile task. Right: Example frames of a participant during the smile and neutral period of the task. (FIG. 27B) Principle Component Analysis (PCA) of the different stimulation conditions (No stim, 50 Hz, and 100 Hz). PCA spaces was calculated over all markers and considered that average features, like amplitude and velocity, for all markers. There was clear separation between the stimulation conditions. Top: Two task PCA examples from SEEG-1. Dots represent individual trials while the while filled circles are the means of each condition. Bottom: SEEG-2's example PCA plots. (FIG. 27C) Box plots of individual kinematic features for SEEG-01.

FIGS. 28A-28C. DBS potentiates MEP in the face during volitional movements. (FIG. 28A) Filtered EMG traces for a single muscle (Cheek) while performing the behavioral task with different stimulation times. Trace according to stimulation condition (a—No Stim, b—VOP 50 Hz, c—VOP 100 Hz). (FIG. 28B) The average EMG trace for the cheek during the exaggerated smile period of the task. Individual trials are the thin lines, the means are the think dark traces, and the standard error through time is the shaded envelope. (FIG. 28C) Box plots of individual MEP features for SEEG-01. For all panels, statistical significance was assessed with two-tail t-test with Bonferroni correction: p<0.05 (*), p<0.01 (**), p<0.001(***).

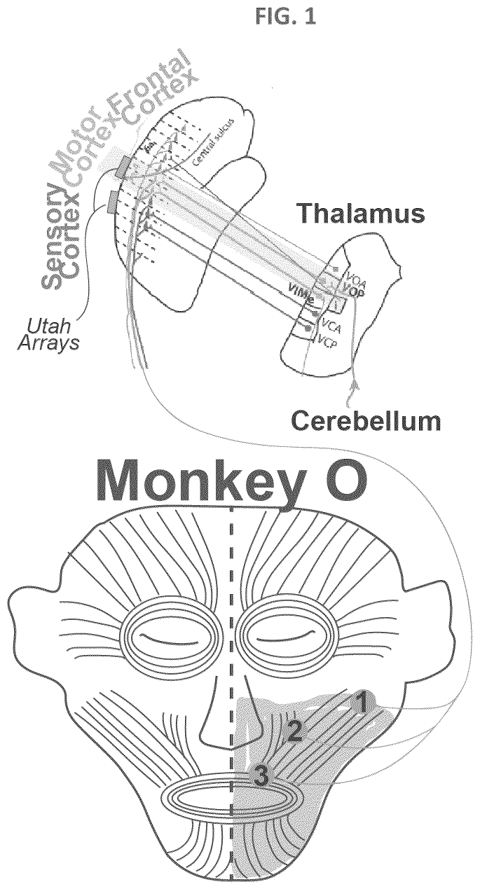

### DETAILED DESCRIPTION

### I. Introduction

Cerebral white matter tract lesions prevent cortico-spinal descending inputs from effectively activating spinal motoneurons, leading to untreatable muscle paralysis. However, in some cases the damage to cortico-spinal axons is incomplete and the spared connections could be potentiated by neurotechnologies to restore motor function. Here, by engaging direct excitatory connections to cortico-spinal motoneurons, deep brain stimulation (DBS) of the motor thalamus is used to facilitate activation of spared cortico-spinal fibers improving muscle activation of the paretic limb. Thalamic DBS in monkeys enhanced motor evoked potentials to arm, hand, and face muscles, as well as grip forces. This potentiation persisted after cerebral white matter lesions. The corresponding thalamic targets in humans (VIM/VOP nuclei) are identified, and replicated the results obtained in monkeys. A DBS protocol is disclosed herein which immediately improved voluntary grip force control in a patient with a chronic traumatic brain injury.

Methods as disclosed herein arise from the unexpected discovery that deep brain stimulation (e.g., continuous stimulation) of specific lateral areas in the thalamus leads to improvements in motor outputs of voluntary movements affected by motor disorders in human subjects. As disclosed herein, stimulation results in faster activation of facial muscles when performing speech therapy exercises than when no stimulation was applied. Further the amplitudes of arm movements and grip strength were increased when the stimulation was applied to the specific thalamic areas of subjects. These findings have broad implications for the improvement of motor impairments in subjects suffering from a motor disorder that affects voluntary movements of the neck, arms, head, and/or tongue.

Using available technology, therapeutic electrical stimuli may be administered to a subject by implanted electrodes under the control of an implanted or external neurostimulator. The electrodes and neurostimulator together comprise a system that may be provided to a subject as a daily assistive device to improve, for example, muscle weakness, muscle control, and/or speech deficits. This system may alternatively be used in combination with motor or speech rehabilitation therapies to improve long-term recovery outcomes. Because treatment methods disclosed herein can comprise electrodes implanted in the brain, they offer the only available approach for treatment of speech impairments that cannot be treated by peripheral neurostimulation, such as spinal cord stimulation or peripheral nerve stimulation. We know of no other system that can be used with this goal.

Methods provided herein are distinguished from conventional methods of thalamic stimulation that have been developed specifically to treat movement disorders such as essential tremor or tremor preponderant Parkinson's disease. Such conventional methods employ higher frequencies of stimulation than those of particular methods disclosed herein (such that a plurality of cerebral cortical areas are stimulated), and they are often insufficient to provide an adequate improvement in a subject's motor function. Particular methods disclosed herein include lower frequencies of stimulation than the foregoing conventional methods. Motor neurons fire at lower frequencies, and those frequencies capable of treating tremors in a subject are not necessarily capable of providing improvements of fine motor control. In examples herein, pre-motor cortex or motor cortex is selectively stimulated by relatively low-frequency stimulation of areas of the thalamus. As shown in the Examples, methods according to disclosed examples provide superior improvement in certain motor outputs associated with motor impairments. For example, methods disclosed herein utilize thalamic stimulation to improve motor outputs of speech disorders for which adequate methods of treatment were not previously available.

### II. Abbreviations

AC anterior commissure
AMT active motor threshold
CST cortico-spinal tract
DBS deep brain stimulation
HDFT high-definition fiber tracking
IC internal capsule
MEP motor evoked potential
MRI magnetic resonance imaging
PC posterior commissure
RMT resting motor threshold
TBI traumatic brain injury
TMS transcranial magnetic stimulation
VA ventral anterior nucleus
VAL ventral anterolateral nucleus
VC ventral caudal nucleus
VIM ventral intermediate nucleus
VLL ventral laterolateral nucleus
VOA ventral oral anterior nucleus
VOP ventral oral posterior nucleus
VPL ventral posterolateral nucleus

### III. Summary of Terms

Unless otherwise noted, technical terms are used according to conventional usage. As used herein, the term “comprises” means “includes.” Although many methods and materials similar or equivalent to those described herein can be used, particular suitable methods and materials are described below. In addition, the materials, methods, and examples are illustrative only and not intended to be limiting. The scope of the claims should not be limited to those features exemplified. To facilitate review of the various examples, the following explanations of terms are provided:

About: As used herein, the term “about” refers to an approximation of a qualitative or quantitative measurement. Whether the measurement is qualitative or quantitative should be clear from its context. With regard to quantitative measurements (for example, the amplitude of a subject's voluntary movements, “about” refers to plus or minus 5% of a reference value. For example, “about” 87 N refers to 87±4.35 N. By way of further example, “about” 0.5% max grip force refers to 0.5±0.025% (87×0.05=4.35).

Closed-loop: A stimulation mechanism wherein a sensor continuously records a feedback signal (for example, a signal correlated or causally linked to a motor deficit in a subject with a motor disorder), and a neurostimulator adjusts parameters of electrode control signals according to the feedback signal. Some examples herein utilize such a closed-loop stimulation mechanism. In specific examples, specific frequency bands, recorded from motor and/or pre-motor areas, trigger application of electrical stimulus via one or more electrodes to thalamic neurons (for example, in the ventral thalamus) including axons projecting to premotor or motor cortex.

Deep brain stimulation: Direct or indirect application of a stimulus to an area within the brain. In specific examples herein, selective deep brain stimulation of neurons in somatotopically and/or stereotactically defined thalamic nuclei is accomplished via electrical stimulation. According to alternative techniques, selective deep brain stimulation of neurons in somatotopically and/or stereotactically defined thalamic nuclei may be accomplished in other examples by optical stimulation via implanted optical fibers, magnetic stimulation, or pharmacological stimulation.

Dysarthria: A speech motor deficit characterized, for example, by an inability of a subject to pronounce words clearly and correctly. Dysarthria can include, for example, the production of slowed and/or slurred speech. As used herein, the term “dysarthria” refers to a speech motor deficit distinct from aphasia. As used herein, “aphasia” refers to a cognitive impairment characterized by, for example, partial or complete loss of speech of a subject, and/or deficits in a subject's understanding of written and spoken word.

Dysphasia: A motor deficit characterized, for example, by difficulty swallowing and weak neck muscles. Dysphasic subjects can require more time and effort to move food or liquid from the mouth to the stomach.

Electrical stimulus: the term “electrical stimulation” refers to the passing of various types of current selectively through one or more electrodes to at least one specific area of a subject's brain (for example, specific areas of the ventral thalamus). An electrical stimulus may selectively stimulate nerves or nerve fibers projecting to premotor or motor cortex at or below a motor threshold of a subject, and/or at or below a perceptual threshold of a subject.

Electrode: An electric conductor through which an electric current can pass. An electrode can also be a collector and/or emitter of an electric current. In some examples, an electrode is a solid and comprises a conducting metal as the conductive layer. Non-limiting examples of conducting metals include noble or refractory metals and alloys, such as stainless steel, tungsten, platinum, iridium, tantalum, titanium, titanium nitride, and niobium. The electrodes can be either interconnected or independently wired.

Implanting: Completely or partially placing a neural probe or device including a neural probe within a subject, for example, using surgical techniques. A device or probe is partially implanted when some of the device or probe reaches, or extends to the outside of, a subject. Implantable probes and devices may be implanted into neural tissue, such as the central nervous system, more particularly the brain, for treatment of different medical conditions and for various time periods. A neural probe or device can be implanted for varying durations, such as for a short-term duration (e.g., one or two weeks or less) or for long-term or chronic duration (e.g., one month, six months, one year, or more), as in a daily assistive device.

Motor impairment: The partial or total loss of function of a body part, for example, limbs, hands, fingers, neck, tongue, mouth, and face muscles. Particular motor impairments include loss of muscle strength, partial paralysis (paresis), loss of dexterity (such as hand finger movement), and uncontrollable muscle tone. As used herein, “motor impairment” specifically includes dysphasia, dysarthria, and speech arrest. A subject can exhibit multiple motor impairments as co-morbidities of a motor disorder.

Motor cortex and pre-motor cortex: As used herein, the term “motor cortex” refers to an area within the cerebral cortex of the brain that is involved in the planning, control, and execution of voluntary movements. The motor cortex is situated within the frontal lobe of the brain, next to the central sulcus. The motor cortex is the only motor control center above the spinal cord that can directly communicate with most of the other motor control structures, such as the thalamus. As used herein, the term “pre-motor cortex” refers to an area located just anterior to the primary motor cortex, which is involved in planning and organizing movements and actions. Neuronal activity in pre-motor cortex precedes activation of the primary motor cortex.

Motor threshold: The minimum thalamic stimulation intensity that can produce a motor output of a given amplitude from a muscle at rest (RMT) or during a muscle contraction (AMT).

Motor disorder: A disorder that comprises a loss of cortical muscle connection in the human subject. In examples herein, a motor disorder may be a speech disorder, a hand/arm motor disorder, or both (where both are independently referred to as a motor disorder). Motor disorders can result from myriad brain injuries; for example and without limitation, ischemic brain injury, hemorrhagic brain injury, traumatic brain injury, brain injury caused by stroke, brain injury caused by intraoperative stroke, a neurodegenerative disorder, Parkinson's disease, essential tremor, dystonia, brain tumor, muscular dystrophy, myasthenia gravis, cerebral palsy, multiple sclerosis, and amyotrophic lateral sclerosis (ALS), transection of eloquent motor and speech related gray or white matter, disorders causing dysarthria and/or dysphagia, and/or any other disorder resulting in discoordination of the oral/deglutition function.

Neurostimulator: A current or voltage-controlled electrical stimulation device. A neurostimulator controls the delivery of an electrical pulse, or pattern of electrical pulses, having defined parameters, for example and without limitation, pulse frequency, duration, amplitude, phase symmetry, duty cycle, pulse current, pulse width, and on-time and off-time. The controlled electrical pulse is delivered through one or more electrodes (for example, leadless electrode(s), or electrode(s) located at the end of a lead, a thin insulated wire) configured to apply the electrical stimulus to the brain of a subject. A neurostimulator may comprise at least one multiple contact lead. Neurostimulators may be utilized to apply a series of electrical pulse stimuli (e.g., charge balanced pulses) through at least one electrode; for example and without limitation, low-frequency pulse train patterns, frequency-sequenced pulse burst train patterns (e.g., wherein different sequences of modulated electrical stimuli are generated at different burst frequencies), and phasic train patterns (e.g., wherein the stimulus control parameters change over the course of feedback from a subject's movement).

Perceptual threshold: The minimum thalamic stimulation intensity necessary for a conscious organism to be aware of a particular sensation.

Subject: Living multi-cellular vertebrate organisms, a category that includes human and non-human mammals, including non-human primates, rats, mice, guinea pigs, cats, dogs, cows, horses, and the like. Thus, the term “subject” includes both human and veterinary subjects.

Treating/Treatment: Preventing, ameliorating, suppressing, and/or alleviating one or more of the symptoms of a subject's motor disorder; for example, as may be determined from one or more motor outputs affected by the subject's motor disorder.

Ventral thalamus: An area of the thalamus comprising the reticular nucleus, the zona incerta, and the ventral lateral geniculate nucleus. As used herein, “ventral thalamus” may refer to a set of particular thalamic nuclei including, for example and without limitation, ventral thalamic nuclei comprising primary thalamic relays for motor and sensory information from the body and head (e.g., the ventral anterior nucleus (VA), the ventral oral posterior nucleus (VOP), the ventral intermediate nucleus (VIM), the ventral caudal nucleus (VC), lateral areas of the VOP and/or VIM (e.g., a lateral area of the VOP and/or VIM associated with arm movements), and medial areas of the VOP associated with face movements). Certain motor and sensory thalamic nuclei herein may be stereotactically defined by reference to one or more the following AC-PC-based stereotactic coordinates: lateral (from about 5 to about 15 mm lateral to the AC/PC line); anterior/posterior (from about 2 to about 10 mm anterior to PC); and dorsal/ventral (from about +1 to about −2 mm from the AC/PC plane.

The motor and/or sensory area(s) of the ventral thalamus includes the ventral anterior nucleus (VA), the ventral oral posterior nucleus (VOP), the ventral oral anterior nucleus (VOA), the ventral intermediate nucleus (VIM), the ventral caudal nucleus (VC), a lateral area of the VOP and/or VIM, a lateral area of the VOP and/or VIM associated with arm movements, and/or a medial area of the VOP associated with face movements.

### IV. Overview

The examples of this disclosure show that DBS of the motor thalamus facilitates the recruitment of cortico-spinal neurons within the motor cortex, which in turn increases motor output in paretic limbs after lesions of the CST. This is promising support for DBS of the VIM/VOP as a therapy to improve motor deficits in people with lesions of the CST.

Identification of optimal DBS stimulation parameters is often guided by trial and error in consequence of the lack of understanding of the underlying neural mechanisms. The examples of this disclosure present experiments aimed at identifying the mechanisms mediating the increase in motor cortex excitability in order to guide the design of optimal stimulation strategies. The examples disclosed herein show that DBS of motor thalamus increased motor output by augmenting the recruitment of cortico-spinal motor neurons within the primary motor cortex via excitatory synaptic inputs from the targeted thalamic nuclei. The 50-100 Hz range was used to sustainably increase motor output. This range is different from commonly used DBS stimulation frequencies (>130 Hz), which is demonstrated here to suppress motor output. A few previous studies had already investigated the use of DBS to treat post-stroke muscle weakness and paresis. However, limited understanding of the underlying mechanisms of stimulation led to suboptimal choices of the implant location and stimulation protocols affecting size and consistency of the observed effects.

DBS of the VIM/VOP facilitated M1 DCS MEPs in the upper extremity muscles via the CST and enhanced grip forces, which suggests that this therapy could be effective in restoring motor functions by addressing two of the main symptoms after damage to the CST: muscle weakness and loss of strength. DBS of the VIM/VOP availed a fully implanted TBI patient to volitionally modulate grip force, demonstrating that continuous DBS improves fine motor control. This method may be applied to stroke- or TBI-induced spasticity as well. Finally, in addition to increasing motor output of hand and arm muscles, DBS of the VIM/VOP induced MEP potentiation of face muscles suggesting that the methods disclosed herein could improve speech motor deficits such as dysarthria and apraxia.

Surprisingly, the observed effects appeared immediately upon turning on thalamic stimulation and stopped as soon as it was turned off, suggesting an “assistive power” of thalamic DBS. DBS-mediated increased motor cortex excitability may have an immediate therapeutic effect with DBS ON and the potential for persistent long-term motor recovery in the absence of DBS stimulation. Further, the assistive effects of DBS may enable patients to engage in exercises that they would not otherwise be able to perform, promoting the advent of DBS-combined behavioral interventions. Finally, the therapeutic effects of VIM/VOP-DBS might be more clinically relevant when compared to non-invasive cortical stimulation because of the higher selectivity and continuous nature of DBS, akin to DBS therapy in patients with Parkinson's disease and Essential Tremor. In fact, non-invasive systems cannot be implanted and consequently be utilized all day. The continuous nature of the DBS stimulation, instead, would enable daily use of the paretic limb possibly driving further plasticity.

Procedure- and hardware-related adverse events of DBS implantation are extremely low (<0.5% of patients). Moreover, surgical risk in patients with a history of stroke or TBI can be minimized by careful pre-operative management of anticoagulants and by delaying the implantation for at least three months following the brain insult.

### V. Deep Brain Stimulation of Specific Ventral Thalamic Nuclei to Treat Motor Disorders

Provided herein are methods for treating a subject (for example, a human subject) having a motor disorder. Motor disorders treatable by methods disclosed herein may result from at least one of ischemic brain injury, hemorrhagic brain injury, traumatic brain injury, brain injury caused by stroke, a neurodegenerative disorder, Parkinson's disease, brain tumor, muscular dystrophy, myasthenia gravis, cerebral palsy, multiple sclerosis, amyotrophic lateral sclerosis (ALS), or transection of eloquent motor-related and/or speech-related gray or white matter. Methods according to particular examples disclosed herein may be utilized to treat (i.e., prevent, ameliorate, suppress, and/or alleviate) a subject's motor disorder in an acute or a chronic phase of the motor disorder. In particular examples herein, a disclosed method is utilized to treat a subject with dysarthria or speech and oral apraxia. Particular examples of methods provided herein may be used to treat stroke subjects that suffer from arm and hand paresis, and/or lost or impaired speech deficits. In particular examples, methods provided herein are used to treat speech and vocalization impairments caused by motor control deficits; for example and without limitation, muscle weakness, dysarthria, dysphagia, apraxia of speech, and speech arrest. These motor control deficits affect about 50% of all acute stage stroke patients, and a third of chronic stage stroke patients.

In some examples herein, stimulation (e.g. continuous stimulation) of motor and sensory areas in the ventral thalamus leads to improvements in vocalization and dysarthria as well as reduced speech arrest. In particular examples, such stimulation results in faster activation of a subject's facial muscles when performing speech therapy exercises than when no stimulation is applied. In particular examples, such stimulation results in increased amplitude of a subject's arm movements, and/or grip strength, than when no stimulation is applied.

In some examples, a method for treating the subject having a motor disorder comprises applying a stimulus (e.g., an electrical stimulus) to neurons in the thalamus, wherein the neurons comprise axons projecting to premotor or motor cortex. The electrical stimulus can be applied with one or more electrodes controlled by a neurostimulator. The electrical stimulus improves at least one motor output associated with the motor disorder. In particular examples, measurements of the motor output are used to adjust the parameters of the electrical stimulus to provide an improvement thereof.

Some examples of methods disclosed herein include the specific stimulation of one or more ventral nuclei of the thalamus, for example, one or more motor and sensory areas of the thalamus (e.g., the ventro-oralis posterior (VOP), the ventral intermediate nucleus (VIM), and/or the ventral oral anterior nucleus (VOA)). In particular examples, stimulation of specific areas of the ventral thalamus targets fibers connecting the thalamus to the pre-motor and motor cortices, thereby increasing the excitability of motor and pre-motor circuits to amplify voluntary motor output to peripheral circuits controlling muscles, for example, such that the stimulation facilitates the subject's natural arm, facial, and/or tongue movements. In particular examples, the stimulation is applied at or below the subject's motor threshold and/or at or below the subject's perceptual threshold. For example, the stimulation may be applied below the motor threshold and below the perceptual threshold; below the motor threshold, but at or above the perceptual threshold; or below the perceptual threshold, but at or above the motor threshold.

In some examples, the electrical stimulus includes electrical pulses defined by parameters including, for example and without limitation, amplitude, pulse width, and pulse frequency. Such electrical pulses may include charge-balanced pulses. In these and further examples, the electrical stimulus may be a continuous electrical stimulus, a closed-loop electrical stimulus and/or a stimulation burst. In particular examples, the electrical stimulus includes electrical pulses with an amplitude of less than about 10 mA; for example, less than 10 mA, less than 9 mA, less than 8 mA, less than 7 mA, less than 6 mA, less than 5 mA, less than 4 mA, less than 3 mA, less than 2 mA, or less than 1 mA. In particular examples, the electrical stimulus includes electrical pulses with pulse widths between about 40 μs and about 2 ms, between 40 μs and 2 ms, between about 60 μs and about 2 ms, between 60 μs and 2 ms, between about 80 μs and about 2 ms; for example, between 80 μs and 2 ms, between 100 μs and 2 ms, between 200 μs and 2 ms, between 300 μs and 2 ms, between 400 μs and 2 ms, between 500 μs and 2 ms, between 600 μs and 2 ms, between 700 μs and 2 ms, between 800 μs and 2 ms, between 800 μs and 2 ms, between 900 μs and 2 ms, between 1 ms and 2 ms, between 1.5 ms and 2 ms, between 80 μs and 1.5 ms, between 100 μs and 1.5 ms, between 200 μs and 1.5 ms, between 300 μs and 1.5 ms, between 400 μs and 1.5 ms, between 500 μs and 1.5 ms, between 600 μs and 1.5 ms, between 700 μs and 1.5 ms, between 800 μs and 1.5 ms, between 800 μs and 1.5 ms, between 900 μs and 1.5 ms, between 1 ms and 1.5 ms, between 1.5 ms and 2 ms, between 80 μs and 1 ms, between 100 μs and 1 ms, between 200 μs and 1 ms, between 300 μs and 1 ms, between 400 μs and 1 ms, between 500 μs and 1 ms, between 600 μs and 1 ms, between 700 μs and 1 ms, between 800 μs and 1 ms, between 800 μs and 1 ms, and between 900 μs and 1 ms. In particular examples, the electrical stimulus includes a pulse frequency between about 20 Hz and about 100 Hz; for example, between 20 Hz and 100 Hz, between 20 Hz and 90 Hz, between 20 Hz and 80 Hz, between 20 Hz and 70 Hz, between 20 Hz and 60 Hz, between 20 Hz and 50 Hz, between 20 Hz and 40 Hz, between 20 Hz and 30 Hz, between 50 Hz and 80 Hz, between 50 Hz and 100 Hz, between 60 Hz and 80 Hz, between 60 Hz and 100 Hz, between 70 Hz and 80 Hz, and between 70 Hz and 100 Hz. In some particular examples, the electrical stimulus includes a pulse frequency of less than 100 Hz, less than 90 Hz, less than 80 Hz, less than 70 Hz, or less than 60 Hz.

In some examples the method includes applying an electrical stimulus to the VIM and the VOP of the thalamus at a pulse frequency of between about 50 Hz and about 80 Hz, to treat a motor impairment of the arms, fingers, or hands causing a loss of muscle strength, partial paralysis, paresis, loss of dexterity, reduced movement, uncontrollable muscle tone, or essential tremor. In some examples, the method includes applying an electrical stimulus to the VOA of the thalamus at a pulse frequency of between about 50 Hz and about 100 Hz, to treat a speech disorder resulting in at least one speech motor impairment. In another example the electrical stimulus may include electrical pulses with an amplitude of between about 1.5 and about 3.5 mA, pulse widths between about 100 μs and about 200 μs, and/or a pulse frequency between about 40 Hz and about 80 Hz.

Accordingly, the electrical stimulus in particular examples herein includes electrical pulses having an amplitude of less than about 10 mA, a pulse width of between about 80 μs and about 2 ms, and a pulse frequency between about 20 Hz and about 100 Hz. In some examples, the electrical stimulus includes electrical pulses having an amplitude of between 0.5 mA and 5 mA, pulse widths between 80 μs and 200 μs, and a pulse frequency between 20 Hz and 80 Hz. For example, the electrical stimulus may include electrical pulses having an amplitude of between about 1.5 and about 3.5 mA, pulse widths between about 100 μs and about 200 μs, and a pulse frequency between about 40 Hz and about 80 Hz.

In some examples, disclosed methods are effected by the use of an implanted neurostimulator that controls the stimulation (e.g., electrical stimulation via one or more implanted electrode(s)) according to predetermined parameters or parameters determined by feedback in a closed-loop system. In particular examples, the one or more electrodes and the neurostimulator comprise a daily assistive device that improves muscle weakness and/or speech deficits. In particular examples, methods disclosed herein can be used in combination with motor or speech rehabilitation therapy to improve long-term recovery outcomes. In some examples, disclosed methods are effected by the use of an implanted neurostimulator that controls the stimulation without feedback, in an open-loop system.

Deep brain stimulation is an advanced neurosurgical procedure involving the implantation of one or more electrode(s) that deliver an electrical stimulus under the control of an externalized or implanted neurostimulator unit. Implantation of the electrode(s), and/or a neurostimulator in examples where the neurostimulator is not externalized, is typically performed by a clinical team including neurologists, neurosurgeons, neurophysiologists, and other specialists trained in the assessment, treatment, and care of neurological conditions. Typically, following selection of an appropriate subject and determination of the area of the subject's brain to be stimulated, precise placement of at least one electrode in the area of the patient's ventral thalamus is carried out in an operating room setting, typically utilizing brain imaging technology and stereotactic targeting made possible by the stereotypical organization of different areas of the thalamus. After administration of local anesthesia, the subject undergoing electrode implantation experiences little discomfort, and is generally kept awake during the implantation procedure to allow communication with the surgical team. In some examples, the methods can be carried out using robotic assistance, as described in Ho et al., 44th Annual International Conference of the IEEE Engineering in Medicine & Biology Society (EMBC) (2022) 3115-18. In some examples, surgical methods such as those described in Sharma & Vavilala, Anesthesiology clinics (2012) 30: 333-46, Mehdi et al., Clin Med (Lond) (2016) 16:535-40, or Dawson, J. et al. The Lancet (2021) 397:1545-53 are used for implantation of the one or more electrodes.

Some examples herein employ an implant that includes one or more electrodes and/or neurostimulator implanted (e.g., fully or partially implanted) in the brain of a subject. Further examples herein employ an implant that includes one or more magnets or optical fibers, and/or a neurostimulator implanted in the brain of a subject.

Numerous types and styles of neural implants (for example, implants including one or more electrodes for providing an electrical stimulus) are available. Any neural implant for specific stimulation of a ventral thalamic area in a subject may be utilized in specific examples. In some examples, more than one electrode is implanted, such as an array of electrodes. In additional examples, a device is provided that can include one or more electrodes. Non-limiting examples include deep brain stimulators, EcoG grids, electrode arrays, microarrays (e.g., Utah and Michigan microarrays), and microwire electrodes and arrays.

In some examples, an implanted neurostimulator can be used for stimulating bio-electric (e.g., neural) signals to motor cortex or pre-motor cortex in a subject. For example, an implanted neurostimulator may be implanted so as to specifically stimulate one or more area of a subject's ventral thalamus for a period of at least 1 month; for example, at least 2, 6, 12, 18, 24, 30, 36, or more months.

In some examples, circuitry is implanted connecting a neurostimulator to one or more electrodes. In particular examples, the circuits are fully implanted (typically in a subcutaneous pocket within a subject's body), or are partially implanted in the subject. The operable linkage of the neurostimulator to the electrode(s) can be by way of one or more leads, although any operable linkage capable of transmitting a stimulation signal from the circuitry to the electrodes may be used in specific examples.

In some examples, electrodes used in accordance with the invention are positioned in specific areas of the ventral thalamus, so as to be capable of selective application of an electrical stimulus to the specific area, by any of the methods conventionally used for positioning of electrodes for deep brain stimulation. The particular procedures used will vary according to the available equipment, training of personnel, and the circumstances of each case. Detailed examples of such procedures are described, for example, in Benabid et al., Movement Disorders 17 (Suppl. 3): S123-129 (2002), and in Schrader et al., Movement Disorders 17 (Suppl. 3): S167-174 (2002). In some examples, the procedures for placement and testing of electrodes are divided into several steps including mounting of a stereotactic ring (also known as a CRW head ring) on the patient's skull, and imaging by high resolution stereotactic commuted tomographic (CT) scanning of the head. The stereotactic CT scan is preferably preceded by high resolution, volumetric, and three tesla magnetic resonance imaging (MRI) in advance of placement of the stereotactic head ring. Planning of the surgical target sites within the brain and trajectories for approach to the selected targets can be achieved using the MRI images and computer software designed for stereotactic targeting, for example, Stereoplan™ Plus 2.3 (Stryker-Leibinger, Friedburg, Germany), and SNS™ 3.14 (Surgical Navigation Specialists, Mississauga, Canada).

Post-operative control of selective electrical stimulation of the thalamic area by the implanted electrode is provided in some examples by a neurostimulator that may be externalized or implanted; for example, subcutaneously (e.g., in the chest or belly of the subject). Following recovery from the implantation, surgery, and connection of electrode leads to the neurostimulator, the subject may be monitored and tested to establish parameters for the electrical stimulation based on the subject's motor disorder and motor impairment; for example, by monitoring one or more motor output(s) that provide a measurement of the extent of the motor impairment and the subject's response to stimulation. In some examples, electrical stimulation by the implanted electrode(s) is delivered to at least one specific area of the subject's ventral thalamus while the subject performs a voluntary activity or task affected by the subject's motor disorder; for example, forelimb tasks (e.g., reaching, grabbing, picking with opposable thumbs, grip squeezing, and fine motor tasks involving precision finger movements) or speech-related tasks (e.g., pronunciation of words and/or syllables, such as “tongue-twisters,” swallowing, production of sounds, facial movement, neck movement, tongue movement, and fine motor tasks involving precision movements of the face, neck and/or tongue).

In some examples, the parameters of the electrical stimulus controlled by the neurostimulator are adjusted according to changes in the one or more motor output(s) that are monitored while the subject performs the specific task, for example, so as to improve the motor outputs, thereby treating the subject's motor disorder. In particular examples, the adjusted neurostimulator is part of a daily assistive device to treat the subject over an extended period of time. In specific examples, the operation of the device and/or the neurostimulator can be at least partially under the control of the subject once the subject is released from a clinical setting. In these and further examples, the subject is taught how to use the device and/or the neurostimulator.

Our method seeks to benefit the chronic stroke patient population that suffers from arm and hand paresis and lost or impaired speech deficits. Specifically, we are targeting the speech and vocalization impairments caused by motor control deficits like muscle weakness, dysarthria, dysphagia, apraxia of speech and speech arrest. These deficits affect about 50% of all acute stage and a third of chronic stage stroke patients. While our method was developed initially for the stroke patient population, this stimulation method could be extended to any population that suffers the above motor control deficits. This extended group could include ALS, Multiple sclerosis, Parkinson's, traumatic brain injury, brain tumors, muscular dystrophy, generalize myasthenia gravis, and cerebral palsy.

### VI. Additional Embodiments

Clause 1. A method for treating a human subject having a motor disorder, the method comprising:
applying an electrical stimulus to neurons in the thalamus, wherein the neurons comprise axons projecting to premotor or motor cortex, and wherein the electrical stimulus is applied with one or more electrodes controlled by a neurostimulator,
wherein the electrical stimulus improves at least one motor output associated with the motor disorder.

Clause 2. The method according to clause 1, wherein the electrical stimulus comprises electrical pulses having an amplitude of less than about 10 mA, pulse widths between about 80 μs and about 2 ms, and/or a pulse frequency between about 20 Hz and about 100 Hz.

Clause 3. The method according to clause 2, wherein the electrical stimulus comprises electrical pulses having an amplitude of between about 1.5 and about 3.5 mA, pulse widths between about 100 μs and about 200 μs, and/or a pulse frequency between about 40 Hz and about 80 Hz.

Clause 4. The method according to any of clauses 1-3, wherein the one or more electrodes are configured to selectively apply the electrical stimulus to axons of the neurons in at least one motor and/or sensory area(s) of the ventral thalamus.

Clause 5. The method according to clause 4, wherein the at least one motor and/or sensory area(s) of the ventral thalamus is selected from the group consisting of the ventral anterior nucleus (VA), the ventral oral posterior nucleus (VOP), the ventral intermediate nucleus (VIM), the ventral caudal nucleus (VC), a lateral area of the VOP and/or VIM, a lateral area of the VOP and/or VIM associated with arm movements, and a medial area of the VOP associated with face movements.

Clause 6. The method according to any of clauses 1-5, wherein the neurostimulator satisfies at least one condition selected from the group of conditions consisting of:
the neurostimulator is externalized;
the neurostimulator is implanted in the subject;
the neurostimulator is implanted in the chest of the subject;
the neurostimulator is implanted in the belly of the subject;
the neurostimulator is fully implanted in the brain of the subject;
the neurostimulator is a daily assistive device or is a component of a daily assistive device;
the neurostimulator is a current or voltage-controlled stimulator;
the neurostimulator comprises at least one multiple contact lead configured to produce orientation selective axonal stimulation; and
the neurostimulator controls the applying of an electrical stimulus comprising at least one of:
   charge balanced pulses,
   a continuous electrical stimulus, and
   a closed-loop electrical stimulus.

Clause 7. The method according to any of clauses 1-6,
wherein the motor disorder is a disorder comprises a motor impairment causing a loss of muscle strength, a speech deficit, dysarthria, partial paralysis, paresis, loss of dexterity, reduced hand movement, reduced finger movement, discoordination of the oral/deglutition function, dysphagia, uncontrollable muscle tone, essential tremor, and/or dystonia, and
wherein the motor disorder results from at least one of ischemic brain injury, hemorrhagic brain injury, traumatic brain injury, brain injury caused by stroke, a neurodegenerative disorder, Parkinson's disease, brain tumor, muscular dystrophy, myasthenia gravis, cerebral palsy, multiple sclerosis, amyotrophic lateral sclerosis (ALS), and transection of eloquent motor-related and/or speech-related gray or white matter.

Clause 8. The method according to any of clauses 1-7, wherein the at least one motor output associated with the motor disorder is a voluntary motor output comprising at least one of:
a motor output associated with hand movement;
a motor output associated with finger movement;
a motor output associated with arm paresis;
a motor output associated with hand paresis;
the amplitude of an arm movement of the subject;
the fine motor control of an arm movement of the subject; and
the amplitude of grip strength of the subject.

Clause 9. The method according to any of clauses 1-7, wherein the motor disorder of the subject is a speech disorder resulting in at least one speech motor impairment.

Clause 10. The method according to clause 9, wherein the at least one speech motor impairment comprises dysarthria, speech arrest, and/or dysphagia.

Clause 11. The method according to clause 10, wherein the at least one speech motor impairment comprises dysarthria.

Clause 12. The method according to any of clauses 9-11, wherein the at least one motor output associated with the speech disorder is a voluntary motor output comprising at least one of:
a motor output associated with a lost speech deficit;
a motor output associated with an impaired speech deficit;
reduced amplitude and/or fine motor control of facial movement;
reduced amplitude and/or fine motor control of neck movement;
reduced amplitude and/or fine motor control of tongue movement;
reduced speech production;
reduced syllabic articulation;
reduced syllabic volume;
increased delay between syllables;
speech arrest;
involuntary repetition of syllables,
prolongation of sounds;
interruptions in speech;
indistinct pronunciation of syllables;
mumbling of syllables;
a motor output associated with dysphasia;
reduced swallowing; and
a motor output associated with painful swallowing.

Clause 13. The method according to any of clauses 1-12, wherein the neurostimulator controls the application of an electrical stimulus in a phasic manner, and wherein the stimulation parameters change over the course of a voluntary movement or while the subject is speaking.

Clause 14. The method according to any of clauses 1-13, wherein the electrical stimulus is applied at or below a motor threshold of the human subject, and/or at or below the perceptual threshold of the human subject.

### EXAMPLES

The following examples are provided to illustrate particular features of certain examples, but the scope of the claims should not be limited to those features exemplified.

### Example 1

### Deep Brain Ventral Thalamic Stimulation Potentiates Motor Output

We leveraged our high-precision technique to position a probe in the posterior limb of the internal capsule (IC) where the corticospinal tract connecting the brain to the spinal cord passes and another in the VOP using MRI-guided stereotaxic surgery in monkeys. We also implanted 96 intracortical arrays in the motor and sensory cortices, a linear probe in the spinal cord at the C6/C7 spinal level, and electromyographic (EMG) leads in upper-limb muscles. We have used this innovative set-up to demonstrate the following characteristics of stimulation of the motor and sensory areas of the thalamus.

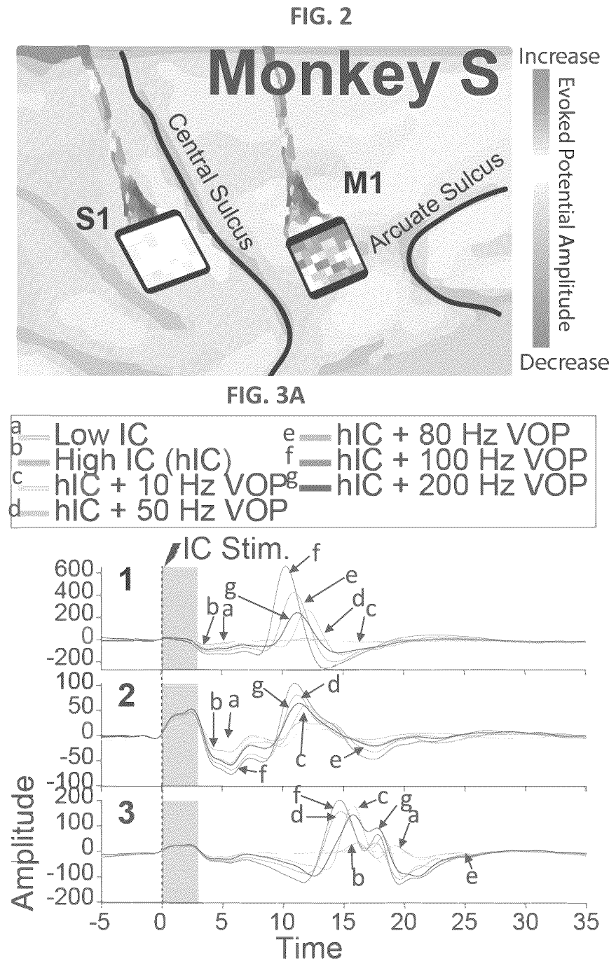

Stimulation increases excitability of the (pre-) motor cortices. We stimulated the VOP at 10 Hz and record evoked potentials from the cortical arrays. We observed a clear cortical evoked potential in the motor cortex (FIG. 2). This demonstrates that stimulation of the thalamus is directly increasing excitability of motor and pre-motor circuits.

Stimulation increases cortico-spinal motor output. We then demonstrated that this increase of the post-synaptic potentials in the (pre)-motor cortices amplifies voluntary motor output to peripheral circuits controlling muscles. For this, we stimulate the internal capsule at 2 Hz to mimic voluntary motor outcome and record EMG and kinematic activity. We then added stimulation of the VOP at different frequencies (10-200 Hz) and amplitudes.

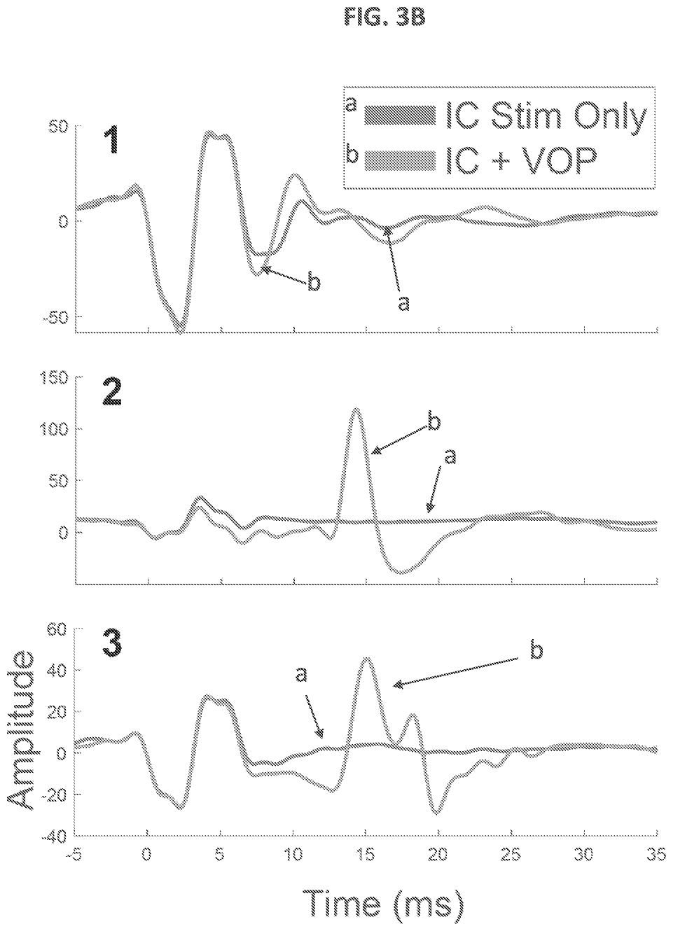

We observed that muscle responses elicited by stimulation of the internal capsule were increased for both the arm and face muscles when the VOP was stimulated (FIG. 3A) proving that stimulation of the VOP increases cortico-spinal motor output. Importantly, this potentiated motor output exhibited a frequency consistent with the internal capsule stimulation, further demonstrating that VOP was only indirectly driving this increase through excitation of the motor cortex and not directly through current leakage to the internal capsule.

We also demonstrated that VOP stimulation increases cortico-spinal motor output when the cortico-spinal projections are destroyed as in the case of neurological conditions (stroke, traumatic brain injury, ALS, etc.). For this, we performed a thermo-coagulation of internal capsule to emulate a stroke. We then stimulated the internal capsule alone or simultaneously to the VOP. We observed that VOP stimulation increased the motor evoked potentials elicited by the internal capsule stimulation also when the fibers were destroyed proving that this technique will be effective in improving motor output in patients with neurological conditions (FIG. 3B).

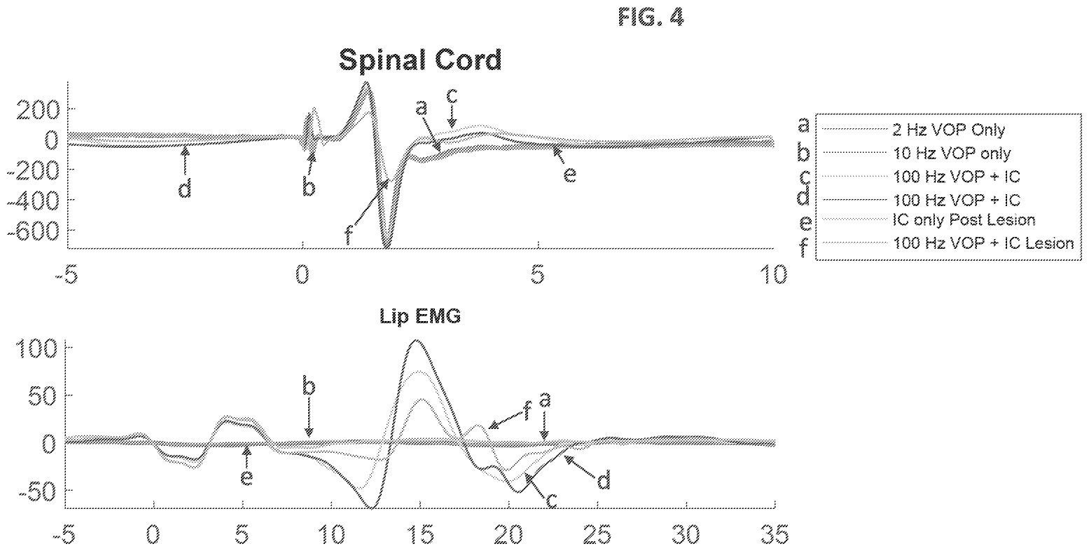

Increased cortico-spinal motor outputs were not caused by antidromic activation of the spinal cord. We used the linear probe implanted in the spinal cord to record spinal activation during VOP stimulation. We observed antidromic volleys in the spinal cord approximately 1.5 ms after stimulation of the VOP, suggesting a potential antidromic recruitment of spino-thalamic axons (FIG. 4). Despite the presence of this antidromic activation in the cord, we did not see any increased motor response within the arm. Indeed, VOP stimulation alone did not result in increased EMG activity regardless of parameter settings. It was only when we simultaneously stimulated VOP and internal capsule that we observed an increase in motor responses. This demonstrates that stimulation of the VOP is increasing voluntary motor control via an indirect increase of the motor cortex excitation.

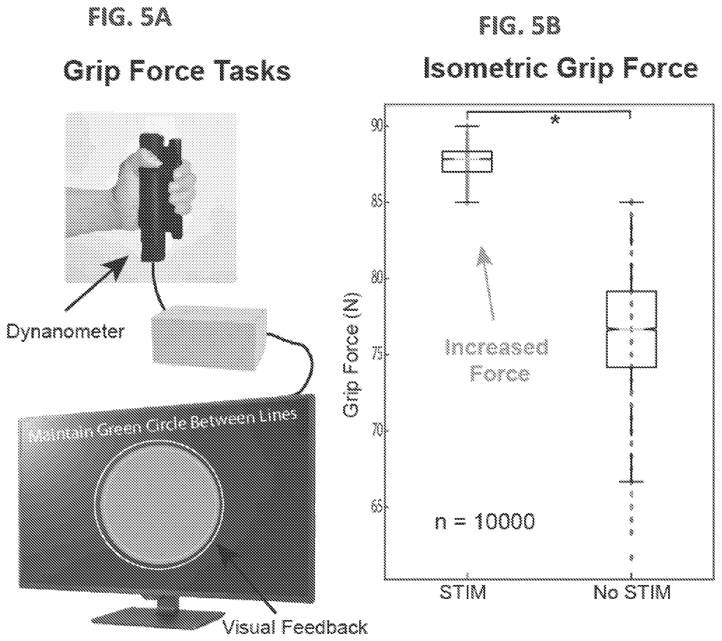

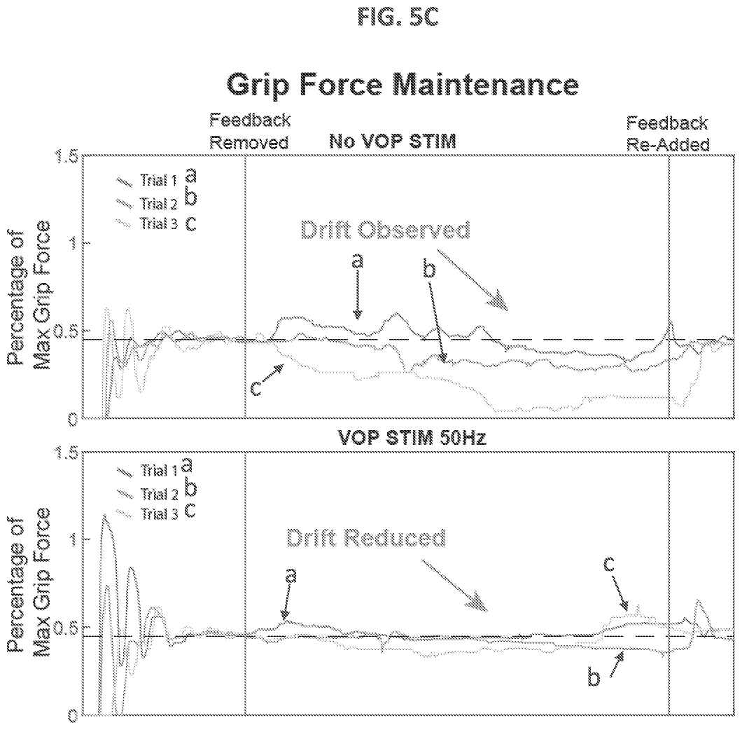

Stimulation potentiates motor output in humans. Following our successful tests in anesthetized monkeys, we recruited n=2 human participants with a history of chronic seizures who are implanted with 10-15 SEEG electrodes in the thalamus and frontal cortical area for seizure monitoring. This procedure is typical for localizing seizure locus and tissue removal and lasts for one to two weeks. These participants had no history of stroke or observed impairments of motor output. We tested whether VOP stimulation increased motor output using two different tasks. In a first task, the subjects were asked to squeeze a grip force dynamometer with their maximum force with and without VOP stimulation (FIG. 5A). We could notice that the force was significantly increase when the stimulation was on (FIG. 5B). In the second task, the subjects were instructed to keep a constant force around 45% of their maximum force while the visual feedback was removed. We could clearly observe that VOP stimulation at 50 Hz prevented a drift in grip force that was visible with no stimulation (FIG. 5C). These results support our hypothesis that VOP stimulation can increase force output and voluntary motor control.

### Example 2

### Deep Brain Ventral Thalamic Stimulation Enhances Speech Production

If VOP stimulation proves beneficial to augment motor output in a specific and reproducible manner in primates, then it could be extended to improving speech production for human patients with compromised corticobulbar tracts, such as in stroke or in traumatic brain injury. As this system is implanted in the brain, it would offer a solution to treat speech impairments that cannot be treated by more peripheral neurostimulations such as spinal cord stimulation or peripheral nerve stimulation. However, to achieve this ambitious goal, we first assessed VOP stimulation in non-speech impaired human participants since non-human primates are unable to produce the vocal responses necessary to determine feasibility.

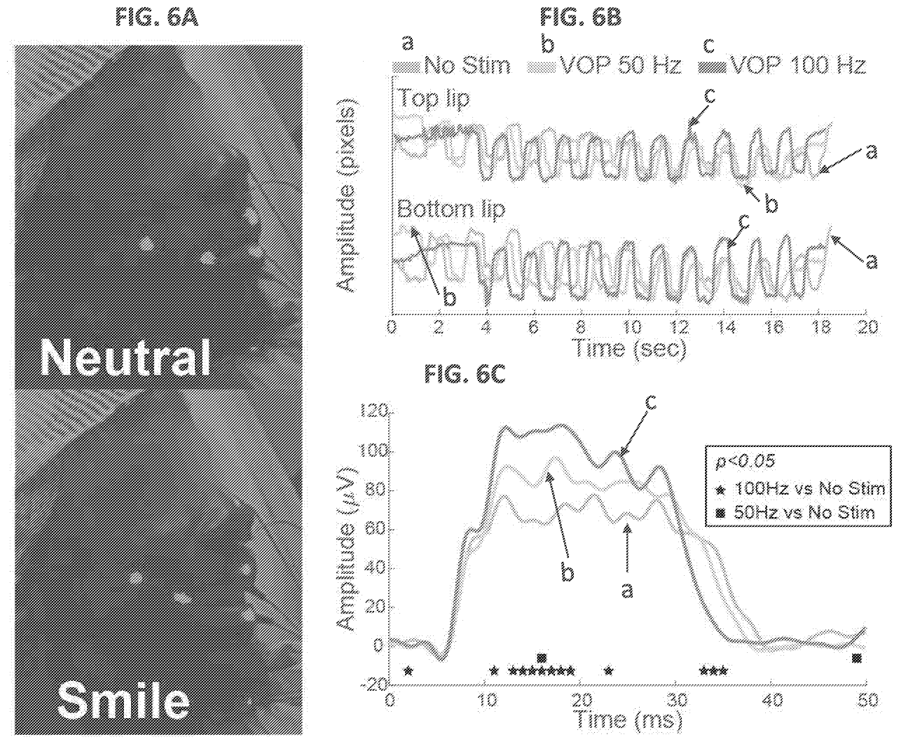

Stimulation increases corticobulbar motor output in human subjects without observed impairments of speech. We recruited human participants with a history of chronic seizures who are implanted with 10-15 SEEG electrodes in the thalamus and frontal cortical area for seizure monitoring. This procedure is typical for localizing seizure locus and tissue removal, and lasts for one to two weeks. These participants had no history of stroke or observed impairments of speech. During the epilepsy monitoring, we collected data from 60-minute sessions of VOP stimulation paradigms. Surface EMGs were used to monitor the contralateral muscles: geniohyoid (tongue), masseter (jaw), and zygomaticus (cheek). Subjects performed speech-therapy exercises that involve moving from an instructed facial expression to a neutral expression as many times as possible in a 20-second window. These tasks were repeated without VOP stimulation and with VOP stimulation at 50 Hz stimulation and 100 Hz. We could accurately track the movements of the mouth using DeepLabCut on the video recordings (FIG. 6A). We could clearly see that during VOP stimulation facial movements (FIG. 6B) and EMG activity (FIG. 6C) were increased in amplitude and more consistent over repetitions.

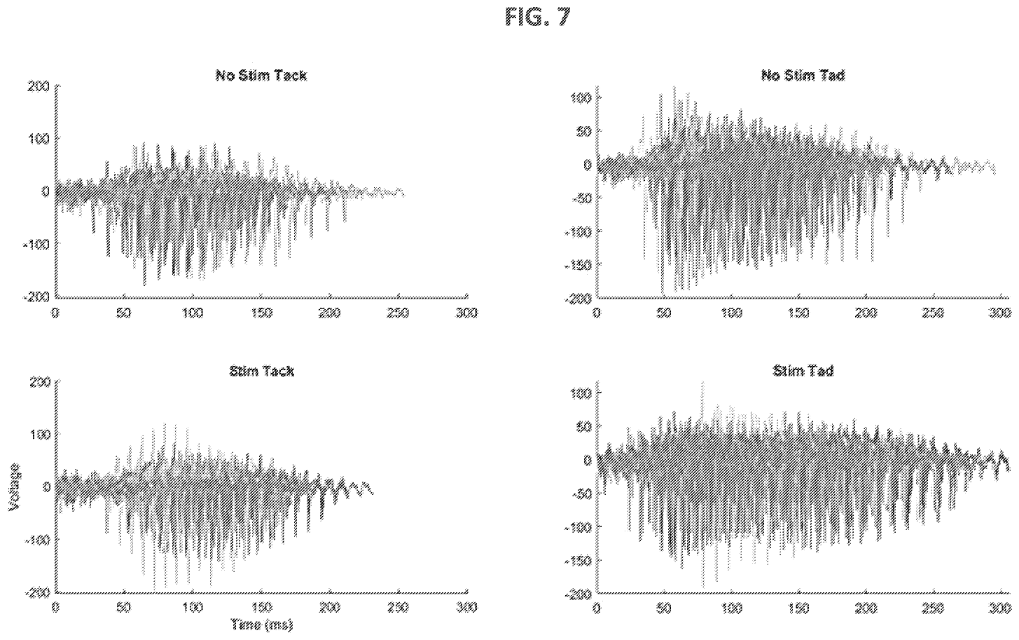

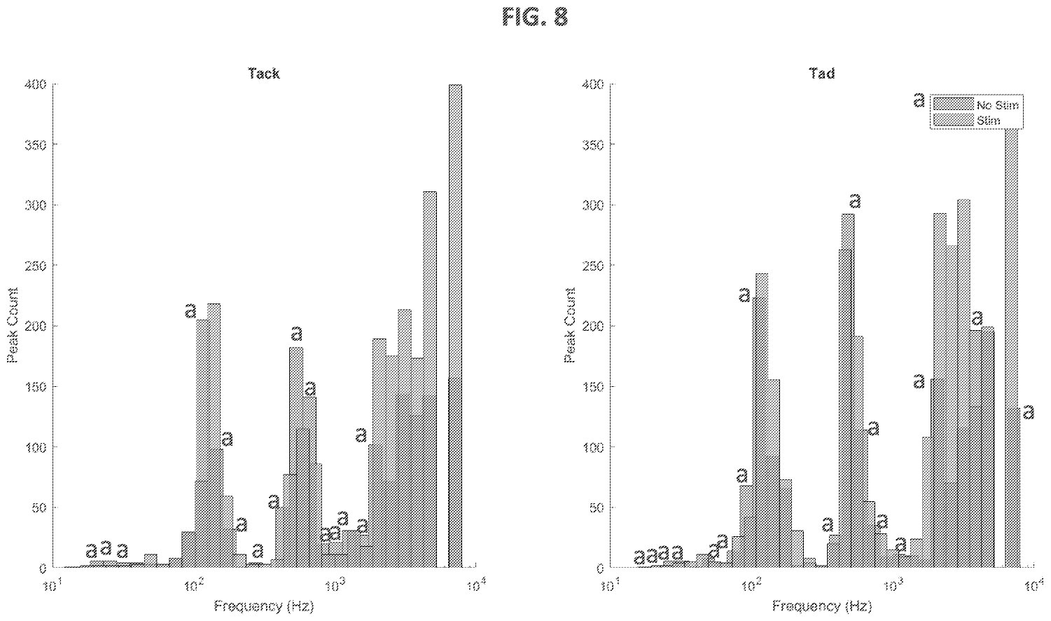

Stimulation increases speech resonance in subjects without observed impairments of speech. Following the speech therapy tasks, subjects were asked to complete a variety of speech articulation tasks. These articulation exercises consist of the subjects repeating two to six syllable phrases that were potential “tongue twisters”. These tasks were performed with and without VOP stimulation. Video and audio recordings of the participants were taken throughout the experiment. We observed that during stimulation the individual syllables sounded louder, faster, and potentially had a slightly improved articulation. We analyzed the trials and found that the speech envelopes were longer when stimulation was ON as compared to stimulation OFF (FIG. 7). The longer speech envelope meant that there was a more stable vowel envelope, which would result in better articulation and reduced slurring. To account for the perceived volume differences, we analyzed the sounds in the frequency domain. We found that during stimulation there was an increase in the production of higher frequencies, i.e. above 1200 Hz (FIG. 8). This increase in higher frequencies results in greater resonance of the syllable, resulting in increased volume. Overall, these results prove that VOP stimulation could help on augmenting articulation, one of the main issues in neurological conditions such as stroke and ALS.

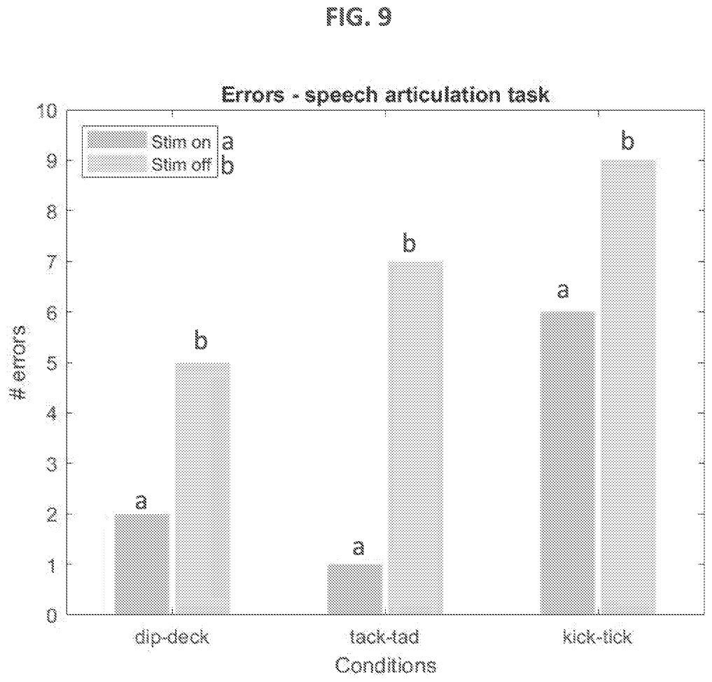

Stimulation enhances speech production in subjects with speech impairments. We recruited one human participant with a history of chronic seizures who was implanted with 10-15 SEEG electrodes in the thalamus and frontal cortical area for seizure monitoring. This procedure is typical for localizing seizure locus and tissue removal, and lasts for one to two weeks. The subject had chronic (i.e., more than 6 months before the test) infarct of the left frontal parietal lobe, resulting in a stroke of the motor cortex speech area with damages to the corticobulbar tracts. This damage manifested in the subject having difficulty in articulating words, experiencing increased syllable slurring during fast repetition, and reduced/impaired breath support. The corticobulbar tract conducts impulses from the brain to the cranial nerves, which control the muscles of the face and neck and are involved in facial expression, mastication, swallowing, and speech articulation. We believe that VOP stimulation is increasing the motor output of these tracts. To demonstrate this, the subject was asked to complete an articular exercise, which consisted of the subject repeating two to six syllable phrases that were potential “tongue twisters” (FIG. 9). These tasks were performed with and without stimulation. Video and audio recordings of the participants were taken throughout the experiment. We analyzed the audio recordings to determine the number of speech errors within a given trial. The list of speech errors we monitored for included stutter, pronounced slurring or swallowing of a syllable, incorrect word replacement, and speech arrest, i.e. difficulty initiating the sound. When we counted the number of errors in the tongue twisters when the stimulation was on and off and we could clearly observe a reduction in the number of errors when the stimulation (50 Hz) was active. These results demonstrate that VOP stimulation enhances speech articulation and so speech production in patients with stroke lesions.

### Example 3

Deep Brain Ventral Thalamic Stimulation with Multicontact Leads

The VOP has a somatotopic and stereotactic organization where more medial areas present neuronal activity more related to face movements and more lateral areas neuronal activity more related to arm and leg movements. Therefore, depending on the desired increased motor output, the stimulation should be selectively oriented towards axons in the more medial or more lateral areas of the VOP. For instance, in order to treat motor deficits of the upper-limb, such as force, the stimulation should be target more to the arm area of the VOP; whereas to treat speech articulation deficits the stimulation should be directed towards the face area of the VOP. For this, the use of recently developed multiple contact leads will be pivotal because different configurations of these electrodes can produce orientation selective axonal stimulation.

### Example 4

### Materials and Methods for Examples 5-14

### Animals

4 Adult Macaca fascicularis (3 male: MK-SC 4 y.o. 5.4 kg, MK-SZ 4 y.o. 5.9 kg, MK-JC 6 y.o. 8.2 kg, 1 Female: MK-OP 6 y.o. 6 kg) and 1 adult male Macaca mulatta (MK-HS 7 y.o. 12 kg) were utilized. When possible, animals were pair housed with another animal prior to any surgical procedures. The animals were not water or food restricted and were given daily enrichments (novel food, toys, puzzles). Detailed information on which animals were involved in specific experimental procedures are reported in Table 1.

TABLE 1
Summary of monkey demographic and experiments performed.

| Animal | | | Age | | |
|---|---|---|---|---|---|
| ID | Species | Sex | (Yr) | Weight | Experiments |
| MK-SC | Macaca | M | 4 | 5.4 kg | Cortical Evoked Potentials/Spike Counts |
| | Fascicularis | | | | |
| MK-SZ | Macaca | M | 4 | 5.9 kg | Cortical Spike Counts |
| | Fascicularis | | | | Kinematic Recording (pre/post lesion) |
| MK-OP | Macaca | F | 6 | 6 kg | Cortical Evoked Potentials |
| | Fascicularis | | | | Antidromic Potentials |
| | | | | | EMG Recordings (pre/post lesion) |
| | | | | | Kinematic Recording (pre/post lesion) |
| | | | | | Radial Nerve Control |
| | | | | | Frequency Analysis |
| MK-HS | Macaca | M | 7 | 12 kg | Cortical Evoked Potentials/Spike Counts |
| | Mulatta | | | | Antidromic Potentials |
| | | | | | EMG Recordings (pre/post lesion) |
| | | | | | Kinematic Recording (pre/post lesion) |
| | | | | | Radial Nerve Control |
| | | | | | Frequency Analysis |
| MK-JC | Macaca | M | 6 | 8.2 kg | Cortical Evoked Potentials |
| | Fascicularis | | | | Antidromic Potentials |
| | | | | | EMG Recordings (pre/post lesion) |
| | | | | | Kinematic Recording (pre/post lesion) |
| | | | | | Radial Nerve Control |
| | | | | | Frequency Analysis |
| | | | | | Force Measurements (pre/post lesion) |

### Animal Surgical Procedure

For each animal, one survival and one terminal surgical procedure was performed. During the survival surgical procedure, five fiducial titanium screws (1.5 mm×4 mm, KLS Martin) were implanted in the skull at non-coplanar depths. A computed tomography (CT) scan (250 μm isotropic, Epica Vimago GT30) of the skull was then performed of the skull with the implanted screws. The procedure was performed using standard aseptic techniques under full anesthesia induced with ketamine (10 mg/km, i.m.) and maintained under isoflurane (1-3%, 2 L/min, inhalant). Over the next 7 days, injected anti-inflammatory drugs once per day (Rymadyl 4 mg/kg, Dexamethsasone 0.4 mg/kg) were injected once per day.

During the terminal surgery the following procedures were performed: 1) peripheral nerve and muscle implantation, 2) robotic deep probe implantation, 3) intra-cortical electrodes arrays implantation, 4) spinal probe implantation, and 5) subcortical lesioning. These procedures were performed under full anesthesia induced with ketamine (10 mg/kg, i.m.) and maintained under continuous intravenous infusion of propofol (1.8-5.4 ml/kg/h) and fentanyl (0.2-1.7 ml/kg/h). Certified neurosurgeons performed these surgical procedures. At the end of the experiments, the animals were euthanized with a single injection of pentobarbital (86 mg/kg) and perfused with 4% paraformaldehyde (1 L/kg) for further tissue imaging.

### Peripheral Nerve and Muscle Implantation

The lateral epicondyle of the arm was dissected around the deep branch of the radial nerve (motor branch) and a cuff electrode (FNC-2000-V-R-A-30 bipolar nerve-cuff Micro-Leads Neuro, Ann Arbor, MI, USA) was implanted. Two branches of the radial nerve were electrically stimulated, and the EMG response was assessed to verify the motor branch from the cutaneous branch. EMG needle electrodes (disposable single 13 mm Subdermal needle electrode, Rhythmlink) were then inserted into into the extensor carpi radialis (ECR), extensor digitorum communis (EDC), flexor digiti minimi (FDM), flexor carpi radialis (FCR), flexor digitorum communis (FDC), abductor pollicis brevis (APB), and biceps (hand and arm muscles). Additionally, in n=3 animals (MK-OP, MK-HS, MK-JC), EMG electrode needles were implanted in the Masseter (Mas), Orbicularis Oris (O. oris), and Buccinator (Buc) face muscles.

### Robotic Deep Probes Implantation

Each depth electrode was implanted electrode using the ROSA One® Robot Assistance Platform (ROSA robot) to allow for highly accurate implantations of the ventral laterolateral (VLL) thalamic nucleus and the hand area of the internal capsule (IC). Prior to the terminal surgery, preoperative T1-weighted magnetic resonance imaging (MRI) scan (400 μm isotropic, TR/TE: 6000/3.7 ms, 7T Siemens whole body human system) and CT imaging were obtained to estimate precise targets and trajectories for each electrode. Both imaging procedures were performed with the animals secured in a customized plastic stereotactic frame to facilitate co-registration. MRI and CT images were then co-registered using ROSA One Brain Application and three targets were selected: the hand area of the cortico-spinal tract (CST) within the IC at the AC-PC level (stimulating electrode), a target 2 cm ventral to create a thermal-ablation lesion (lesioning electrode), and the VLL nucleus of the motor thalamus. The trajectories of the probe were planned in the ROSA software, avoiding vasculature and the ventricles. The entrance of the probes was positioned 2 cm in front of the central sulcus to keep the motor and somatosensory cortices intact for the implantation of the intra-cortical electrode arrays. Monkeys were then placed in a prone position in a stereotaxic head frame (Kopf, Model 1530, Tujunga, CA, USA) and the ROSA robot was registered to the monkey using the implanted fiducial screws. The robot guided the drilling of penetration holes and the precise implant of the fixation bolts. For the two probes within the internal capsule, a radiofrequency cannula (S-100 5 mm ActiveTip Straight cannula 22G, Abbott) and a radiofrequency electrode (RF-SE-10 Reusable Stainless Steel Electrode, Abbott) were inserted and fixed at the correct depth into the brain. Correct positions of the probes within the IC were estimated by recording evoked electromyography (EMG) potentials from stimulation of the IC at 2 Hz at amplitudes between 800 uA and 2 mA that should produce mono-synaptic activation of the cervical motoneurons. For the stimulation of the VLL, a 16-channel Dixi electrode (DIXI Microdeep® SEEG Electrodes) was inserted into its fixation bolt. Correct position within the VLL was confirmed by recording evoked potentials in the cortex during electrical stimulation (1 Hz) at amplitudes between 1 and 4.8 mA that elicited clear evoked potentials in the motor cortex, but not in the somatosensory cortex. Detailed information on the amplitudes and pulse durations used for stimulation of the IC and the VLL are reported in Table 2.

TABLE 2
Simulation parameters for IC, VLL, and radial nerve.

| | IC Stimulation | VLL Stimulation | VLL Burst | Radial Nerve |
|---|---|---|---|---|
| | Parameters | Parameters | Parameters | Stimulation Parameters |
| Animal | (Amplitude, | (Amplitude, Pulse | (Amplitude, | (Amplitude, Pulse |
| ID | Pulse Width) | Width) | Pulse Width) | Width) |
| MK-SC | — | 4.8 mA, 2 ms | — | — |
| MK-SZ | 800 μA, 800 μs | 1 mA, 2 ms | — | — |
| MK-OP | 2 mA, 3 ms | 1.5 mA, 100 μs | 1 mA, 100 μs | 120 μA, 100 μs |
| MK-HS | 1 mA, 1 ms | 2 mA, 300 μs | 2 mA, 300 μs | 90 μA, 100 μs |
| MK-JC | 2 mA, 1 ms | 2 mA, 300 μs | 2 mA, 300 μs | 105 μA, 100 μs |

— means that that stimulation was not performed in that particular animal

### Intra-Cortical Electrodes Arrays Implantation

A 20 mm diameter craniotomy was performed over the central sulcus and dura was removed to expose primary motor and primary somatosensory cortices. Functional motor areas of the arm were identified through anatomical landmarks and intra-surgical micro-stimulation. The position of the primary somatosensory area (S1) was then determined in relation to the hand representation of the primary motor cortex (M1). Intracortical arrays in M1 (48 channels) and S1 (48 channels) were then implanted for a total of 96 channels (400 μm pitch, electrode length 1.0 mm Blackrock Microsystems, Salt Lake City, UT, USA). In MK-JC, a 64-channel array was implanted into M1 and a 48-channel array into S1 (total 112 channels, 400 μm pitch, electrode length 1.0 mm Blackrock Microsystems, Salt Lake City, UT, USA). The arrays' implantation was achieved using a pneumatic compressor system (Impactor System, Blackrock Microsystems).

### Spinal Probe Implantation

After the completion of the cranial surgery, a laminectomy was performed from C3 to T1 vertebrae exposing the cervical spinal cord. A 32-channel linear spinal probe was implanted (A1×32-15 mm-100-177-CM32 Linear Probe with 32 pin Omnetics Connector, NeuroNexus, Ann Arbor, MI, USA) in the gray matter at the C6-C7 spinal segment to record spinal local field potentials and multi-unit spikes. To implant the probe, the dura mater was opened and a small hole was placed in the pia using a surgical needle through which penetration of the probe with micromanipulators was possible. The probe was implanted using Kopf micromanipulators (Kopf, Model 1760, Tujuna, CA, USA).

### Subcortical Lesioning

A radiofrequency generator (NeuroTherm NT 1100) was utilized to create the lesions using the methods of Vinas, F. et al. Stereotactic and functional neurosurgery (1992) 58, 121-133. Time and temperature parameters used for the lesion for each animal are summarized in Table 3. At the end of the experiments before perfusion a small lesion (60° C. for 5 seconds) was created in VLL through the Dixi electrode to visualize the implant location post-mortem.

TABLE 3
Temperature and time used to create a lesion
of the CST within the internal capsule.

| Animal ID | Temperature | Time |
|---|---|---|
| MK-SC | 90° C. | 60 seconds |
| MK-SZ | 75° C. | 60 seconds |
| MK-OP | 80° C. | 60 seconds |
| MK-HS | 80° C. | 60 seconds |
| MK-JC | 80° C. | 30 seconds |

### Data Acquisition and Electrophysiology

Stimulation of the IC, VLL, and radial nerve was provided using an AM stimulator (model 2100 A-M Systems, Sequim, WA, USA). Stimulation was delivered as either single pulses or bursts of cathodic, charge balanced, symmetric square pulses. The stimulation intensity for the IC and the nerve was set for each animal at the motor threshold (i.e., when movements became visible). Detailed information on the amplitudes and pulse widths used for stimulation of the IC, the VLL, and the radial nerve are reported in Table 2. All electrophysiological and neural data was amplified, digitally processed, and recorded using the Ripple Neuro Grapevine and Trellis software at a sampling frequency of 30000 Hz. Neural events were determined for each channel of the Utah arrays and linear probes by applying a broadband filter between 300 Hz and 3 kHz and setting a voltage threshold of 3 root-mean-square. LFPs were filtered between 0 and 500 Hz and then downsampled to a sample frequency of 1000 Hz.

### Grasp Force Data Acquisition

To produce a grasp motion from an anesthetized monkey (MK-JC), the IC was stimulated every two seconds with a 1-second pulse train at 47 Hz and 1 mA. Grasp force data was collected using a 6-axis low-profile force and torque (F/T) sensor (Mini40, ATI Industrial Automation, North Carolina). The sensor was powered using a multi-axis (F/T) transducer system (ATI DAQ F/T) that also calibrated the force data. The sensor was able to measure three degrees of force (+/−810 N for Fxy, +/−2400 N Fz) and torque (+/−19 Nm Txy, +/−20 Nm Tz) simultaneously. A small rod was mounted to the xy-plane of the sensor to be a grip handle for the animal and the system was secure under the animal's left arm. The force and torque data were digitized and recorded using the Ripple Neuro Grapevine and Trellis software at a sampling frequency of 30000 Hz.

### Animal Post-Mortem High Definition Fiber Tracking (HDFT)

After perfusion, the brain was dissected for post-mortem T2-weighted MRI (125 μm isotropic resolution, TR/TE: 1500/60.57 ms) and diffusion-weighted MRI (0.5 mm isotropic resolution, b-values=1800, 3400, and 5200 s/mm², with 30, 40, and 90 diffusion directions, respectively, and 6 A₀ images). Image data were acquired using a 9.4 T/31 cm horizontal-bore Bruker AV3 HD animal scanner equipped with a high-performance 12-cm gradient set, capable of 660 mT/m maximum gradient strength, and a 72 mm quadrature birdcage RF coil. Diffusion tensor estimation and tractography were performed using DSI studio. The accuracy of b-table orientation was examined by comparing fiber orientations with those of a population-averaged template using the methods of Yeh, et al. NeuroImage (2018) 178:57-68. The restricted diffusion was quantified using restricted diffusion imaging using the methods of Yeh, et al. Magnetic resonance in medicine (2017) 77:603-12. The diffusion data were reconstructed using generalized q-sampling imaging (using the methods of Yeh, et al. IEEE transactions on medical imaging (2010) 1626-35) with a diffusion sampling length ratio of 0.6.

A tracking threshold of 0, angular threshold of 0, and a step size of 1 mm was used for fiber tracking to characterize the thalamocortical projections. Tracks with lengths shorter than 20 mm or longer than 200 mm were discarded. A total of 10,000 tracks were placed. Topology informed pruning (as described in Yeh, et al. Neurotherapeutics (2019) 16:52-58) was applied to the tractography with 2 interactions to remove false connections. Each of three regions of the monkey motor thalamus (the ventral anterolateral region (VAL), ventral laterolateral region (VLL), and ventral posterolateral region (VPL)) were selected as a seed to create tracks that projected to the cortical areas of interest: the primary somatosensory cortex (S1), the primary motor cortex (M1), the dorsal premotor cortex (PMd), and the supplementary motor area (SMA). Cortical and subcortical regions were mapped using the built-in primate CVIM atlas (described in Calabrese et al. Neuroimage (2015) 117:408-16) and confirmed by certified neurosurgeons. The volume of the tracts was calculated from projects of each motor thalamic region to each cortical region. The volume of each tract was then normalized by the total volume of projections from all motor thalamic regions to all cortical regions.

A receiver operating characteristic (ROC) curve was computed to analyze the selectivity of projections to only M1 from each of the motor thalamus nuclei comparing the true positive rate and false positive rate of projections to M1 compared to those not to M1. Both rates were calculated while thresholding at each unique normalized volume. The area under the ROC curve was computed for the VPL, VLL, and VAL.

A region based on the size of the VLL thermal-ablation lesion was manually drawn to quantify the projections from the area of stimulation of the VLL DBS electrode. This region was then mirrored to the intact contralateral side (due to disruption of thalamocortical fibers by the lesion) and selected as the seed. S1, M1, PMd, and SMA were selected as regions of interest. The same tracking parameters were used as above. The volume of each tract normalized by the total volume of tracts from the area of stimulation to all cortical areas was then calculated.

A tracking threshold of 0, angular threshold of 0, and a step size of 0 mm to quantify the damaged cortico-spinal tract (CST). Tracks with lengths shorter than 30 mm or longer than 500 mm were discarded. A total of 10,000 tracks were placed. Topology informed pruning was applied to the tractography with 2 interactions to remove false connections. Seed regions were manually drawn in the left and right cerebral peduncles and the respective left and right motor cortices as regions of interest to reconstruct the CST and confirmed by a neurosurgeon. The volume of each CST was then calculated and normalized by the combined volume of the left and right CST.

### Analysis of Cortical Activity

The broadband cortical data was first bandpass filtered between 10 and 5000 Hz with a 3rd order Butterworth filter and 1 ms blanking was applied over stimulation artifacts. A 25 ms window (5 ms before stimulation, 20 ms after stimulation) was then extracted to capture the entirety of the cortical evoked potential. The peak to peak amplitude was then calculated and averaged across all stimulation trials. Multi-unit activity was quantified offline by calculating the average spike count across all trials for each channel. Cortical spikes were detected with a threshold of 3-3.5 (MK-SC: 3, MK-SZ: 3, MK-OP: 3.5, MK-HS: 3, MK-JC: 3) standard deviations above baseline for each channel of the 2 Utah arrays. The average spike counts were baseline corrected and were calculated using a bin size of 2 ms. Stimulation artifacts were removed by blanking 13 ms after stimulation.

Local field potentials were recorded in the M1 Utah array when stimulating the IC at 2 Hz to analyze antidromic potentials to IC stimulation. A 100 ms 100 Hz burst of stimulation was then sent to the VLL preceding a single pulse of IC stimulation at various delays (2 ms, 5 ms, 10 ms, 50 ms). The same filtering was applied to the cortical data as above. A 45 ms window was extracted (5 ms before IC stimulation, 40 ms after IC stimulation) to capture the entire antidromic potential. The peak to peak amplitude of the antidromic potential was then calculated and averaged across all stimulation trials.

Bandpass filtered cortical evoked potentials over M1 had 12 ms windows extracted (2 ms before stimulation, 10 ms after stimulation) for each stimulation pulse at a variety of VLL frequencies (VLL stim 10, 50, 80, and 100 Hz) to calculate the frequency dependent cortical responses to VLL. The peak to peak amplitude of the evoked potential over the time range of 1.5 to 10 ms after the simulations was calculated and averaged across all stimulation trials.

### Analysis of Muscle Activity and Kinematics

Electromyographic activity was bandpass filtered between 30 and 800 Hz with a 3rd order Butterworth filter. Stimulation triggered averages (window from 5 ms to 25 ms after IC stimulation) of motor evoked potentials were then computed and the peak to peak amplitude for each pulse of IC stimulation and each muscle were calculated.

Kinematics were recorded by a GoPro® Camera and analyzed through Deeplabcut (described in Mathis, A. et al. Nature neuroscience (2018) 21:1281-89), a neural network for 2D and 3D markerless pose estimation based on transfer learning. A subset of video frames (40-80 frames) per condition were selected by Deeplabcut to capture diversity of the movement. The thumb, index finger, wrist, and elbow were manually labeled in each of those frames. Deeplabcut was used with the labeled frames to create a training network by merging all extracted labeled frames. The trained network was used to analyze all the videos and extract the kinematic traces for each of the labeled body points. Peak to peak amplitudes in pixels of each joint for each pulse of IC stimulation were calculated.

### Analysis of Frequency Dependent Modulation of Muscle Responses

For each animal, muscle, and IC paired VLL stimulation (10, 50, 80, 100, and 200 Hz) protocol all MEP responses from each IC stimulation pulse (window from 0 ms to 25 ms after IC stimulation) were concatenated. These MEP traces were visually inspected and characterized according to five criteria: 1) “no potentiation”—the responses are consistent with IC alone stimulation, 2) “initial adaptation”—the responses at the start of the stimulation train are variable and then become potentiated, 3) “potentiation”—the responses are increased as compared to IC alone, 4) “attenuation”—the responses decrease through time, 5) “suppression”—the responses are suppressed through time. In order to ensure that the classifications were not biased by the muscle type or stimulation protocol, the MEP traces were unlabeled and presented in a random order during the visual inspection. The probability distribution over all muscles and animals was calculated for each unique stimulation protocol.

### Analysis of Spinal Activity

The same filtering and extraction process was applied to the spinal evoked potentials as to the cortical evoked potentials (filter 10-5000 Hz 3rd order Butterworth and 25 ms windows). The peak to peak amplitude and response onset within the first 5 ms following VLL stimulation were calculated across all channels of the intraspinal probe to identify the antidromic response from the VLL stimulation. The ventral, intermediate, and dorsal areas were distinguished on the probe using the intraspinal probe map. The peak to peak amplitude by the maximum value for each animal was normalized for each animal independently to compare the responses across animals. The IC stimulation artifact was blanked from 250 us before stimulation to 500 us after the stimulation pulse ended when assessing the frequency dependent effects of VLL stimulation paired with IC stimulation on the spinal responses. The area under the curve (AUC) of the spinal evoked potentials was then calculated for each VLL stimulation protocol (10, 50, 80, and 100 Hz). The AUC were calculated from 5 to 10 ms after IC stimulation to exclude any stimulation artifacts and secondary post-synaptic responses. The AUC were compared against the AUC of the spinal responses from IC stimulation alone for potentiation or attenuation.

### Human Participants

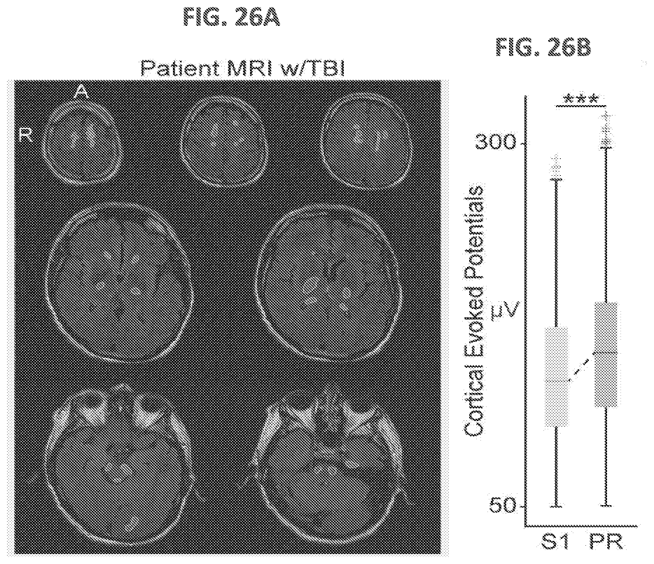

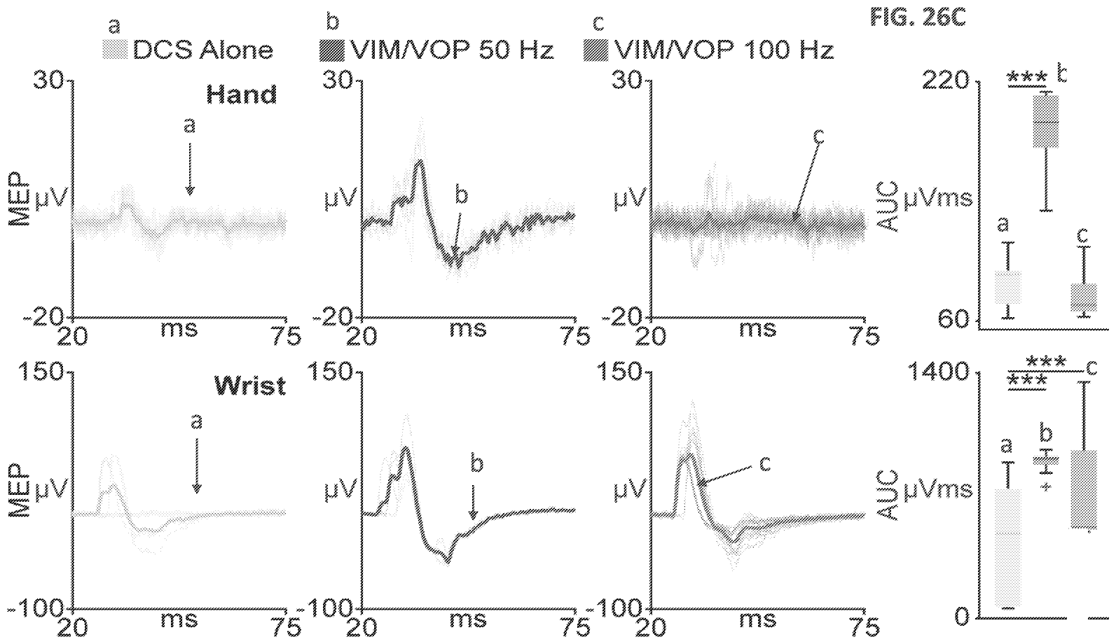

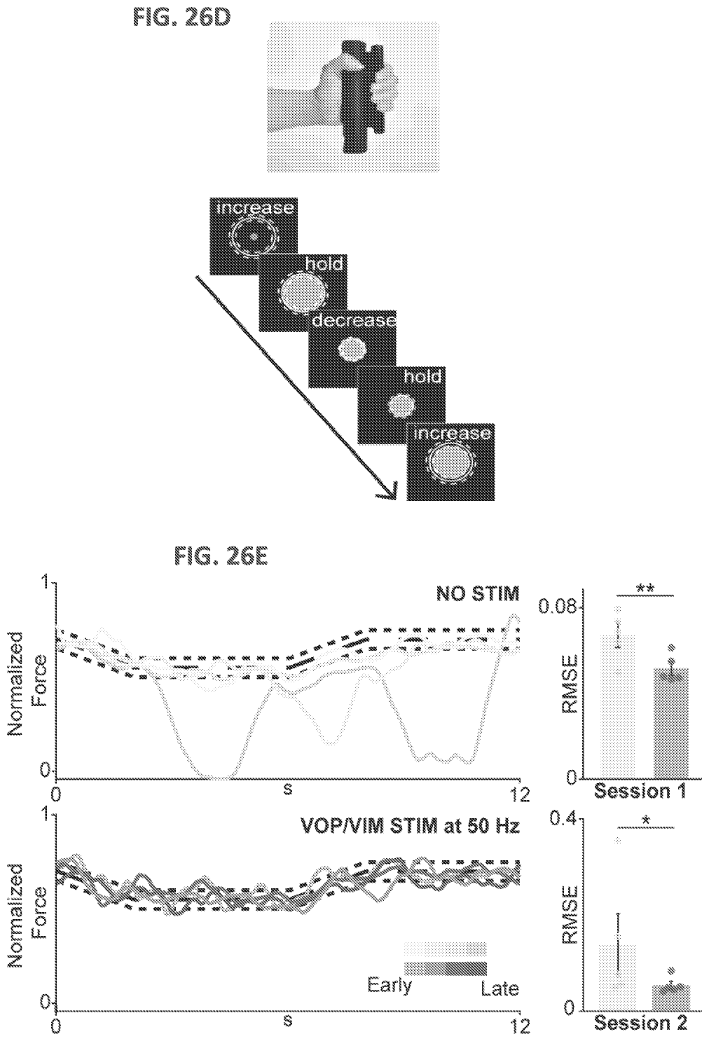

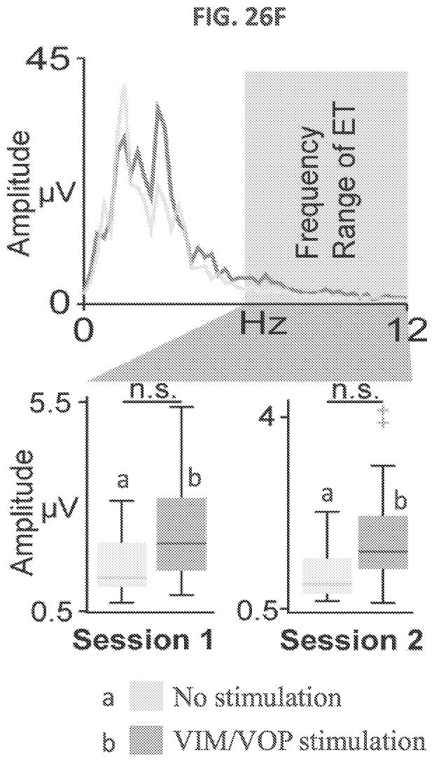

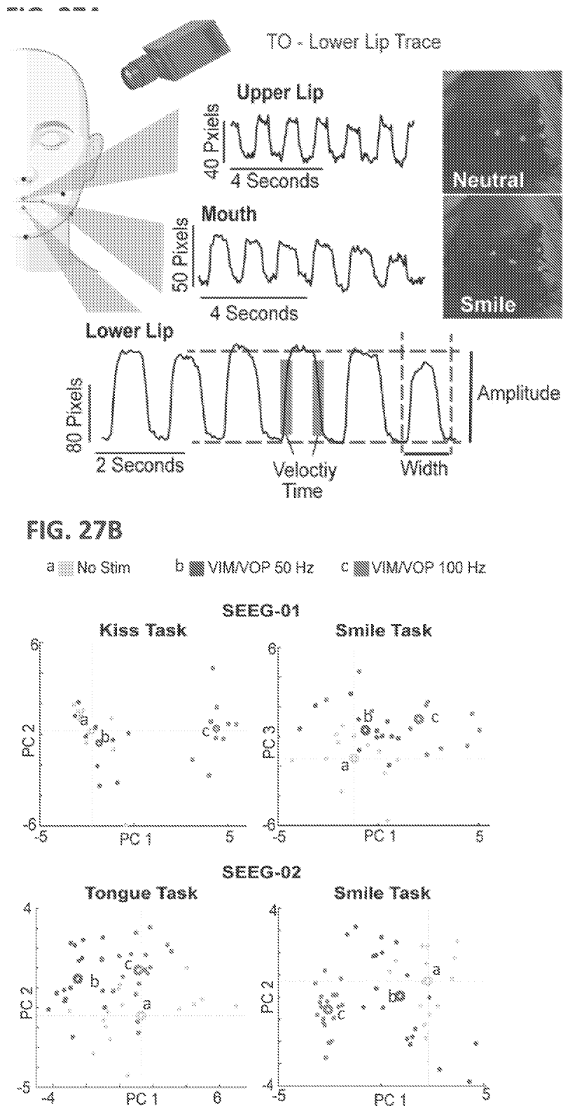

Electrophysiological experiments were performed on n=4 human subjects (2 males and 2 females) of age 69.5±8.54 (mean±std) who presented with medically-intractable asymmetric Essential Tremor (ET) symptoms and were undergoing DBS implantation of the VIM/VOP nucleus (ventralis intermediate nucleus/ventralis oralis posterior), which corresponds to the VLL nucleus in monkeys. The least symptomatic arm was tested to better approximate normal function. Additionally, a traumatic brain injury (TBI01) patient (male in his 40's) was tested who had suffered subarachnoid hemorrhaging from a motor vehicle accident. This hemorrhage led to multiple foci of traumatic infarcts in both frontal lobes (FIG. 26A, more pronounced in the left than the right hemisphere) and evidence of diffuse axonal injury involving the CST and bilateral edema of the cerebral peduncles and pons. Consequently, TBI01 suffered from hemiparesis of the right side and left upper extremity muscle weakness and tremor. The patient was recommended for bilateral DBS electrode implant to treat the post-traumatic tremor.

### Human Intraoperative Data Acquisition and Electrophysiology

Pairs of needle electrodes (Rhythmlink) were implanted subcutaneously in the deltoid, biceps brachii, triceps brachii, flexors (flexor carpi ulnaris), extensor (extensor carpi radialis longus), and abductor pollicis brevis (APB) to record MEPs. Prior to DBS insertion, a subdural strip electrode (6 contact platinum subdural electrode, AD-TECH Medical Instrument Corporation, Oak Creek, WI) was implanted over the cortical surface with verification of positioning provided using median nerve somatosensory evoked potential (SSEP) phase reversal (PR) mapping to locate the hand representation of S1 cortex and the approximate location of the central sulcus as described in Cedzich et al., Neurosurgery (1996) 38: 962-70. The electrode position was adjusted using direct cortical stimulation (DCS) and recording of DCS MEPs to contralateral upper extremity muscles. The contact was located over the precentral gyrus that generated the largest amplitude MEP in the hand muscle (APB). DCS of the hand representation of M1 was provided using trains of 5 stimulation pulses (0.5 ms) at 400 Hz every two seconds at amplitudes up to 15 mA using an intraoperative neurophysiological monitoring system (XLTEK Protektor, Natus Medical). Detailed information on the stimulation parameters used for DCS for each subject are reported in Table 4. After implantation of the subdural strip electrode, the surgery continued following standard clinical procedures and DBS electrodes were implanted in the VIM/VOP nucleus bilaterally using subcortical mapping techniques. The electrophysiology experiments were performed following completion of the clinical procedure, with the subject maintained under anesthesia with propofol. Specifically, DCS MEPs from stimulation of the optimal electrode contact over the hand representation of the primary motor cortex were recorded without and with continuous stimulation of the VIM/VOP nucleus (DBS contacts −1 +8) at 50, 80, and 100 Hz with pulses of 100 us and amplitude of 3 mA. MEPs were collected for 100 ms trials following each DCS stimulation burst and recorded using the XLTEK system at a sampling frequency of 6000 Hz. Stimulation of the VIM/VOP was delivered via the Boston Scientific clinician programmer that connects via Bluetooth to an external trial stimulator. The same DBS electrode was used to generate cortical evoked potentials in the subdural strip electrode that were recorded using the Ripple Neuro Grapevine and Trellis software at a sampling frequency of 30000 Hz.

TABLE 4
Stimulation parameters for DCS and VIM/VOP in humans.
The amplitude of stimulation of the VIM/VOP was
always at 3 mA and within contact −1 and +8.

| Subject | DCS amplitude | VIM/VOP frequency |
|---|---|---|
| S01 | 8 mA, 9 mA, 10 mA | 50 Hz, 100 Hz |
| S02 | 13 mA, 14 mA, 15 mA | 50 Hz, 80 Hz |
| S03 | 7 mA, 11 mA | 50 Hz, 80 Hz, 100 Hz |
| S04 | 8 mA, 10 mA, 12 mA | 50 Hz, 80 Hz, 100 Hz |

### Behavioral Experimental Recordings and Analysis

To measure the level of voluntary force control, the participant was asked to grasp a force dynamometer (Pinch/Grip Digital Myometer, MIE Medical Research, Yorkshire, UK), which recorded the force in newtons at a sampling rate of 30000 Hz. A task was prepared where the participant modulated their force between two limits (55-70% of maximum voluntary contraction measured with DBS OFF). A screen provided feedback of the target force and the participant's applied force. The root-mean-squared error between the applied force and the expected force was calculated at each time point. To calculate the frequency power of the force trace, filtration was performed with a band-pass 2nd order Butterworth filter (cut-off frequencies of 2-5000 Hz) and a fast fourier transform was performed of the hold portion of the force task. The average power from 6-12 Hz (frequency range of Essential Tremor) during VIM/VOP stimulation on and off was compared.

### Analysis of Intraoperative Cortical and Muscle Activity

Electrocorticographic data were filtered with a band-pass 2nd order Butterworth filter (cut-off frequencies 10-500 Hz) and a 60 Hz notch 2nd order Butterworth filter. The stimulation artifact was blanked. Epochs of 30 ms (10 ms before and 20 ms after the stimulus onset) were extracted and stimulation triggered averages were computed. The peak to peak amplitude of each cortical evoked potential was calculated as the difference between the maximum and minimum voltage value in the interval 3-15 ms following the stimulus onset.

To quantify DCS MEPs recorded from triggered electromyographic activity, stimulation triggered averages (EMG from 10 ms to 75 ms after stimulation) were computed and the AUC for each muscle was calculated.

### Human HDFT, Volume of Activation, and Lesion Segmentation

### HDFT

The diffusion images were acquired on a SIEMENS Prisma Fit scanner using a diffusion sequence (2 mm isotropic resolution, TE/TR=99.2 ms/2490 ms, 257 diffusion sampling with maximum b-value 4010 s/mm²). The accuracy of b-table orientation was examined by comparing fiber orientations with those of a population-averaged template as described in Yeh et al. NeuroImage (2018) 178:57-68. The tensor metrics were calculated using DWI with b-value lower than 1750 s/mm².

For fiber tracking, a tracking threshold of 0, angular threshold of 50, and a step size of 1 mm were used. Tracks with lengths shorter than 30 mm or longer than 500 mm were discarded. A total of 10,000 tracks were placed. Topology informed pruning was applied to the tractography with 2 interactions to remove false connections. To quantify the projections from the area of stimulation of the VIM/VOP DBS electrode, a region at the stimulation contact was manually redrawn and considered as the seed region. S1, M1, PMd and SMA were the regions of interest. The volume of each tract normalized by the total volume of tracts from the area of stimulation to all cortical areas was then calculated.

### Volume of Activation

After DBS implantation of the VIM/VOP DBS electrode, patients underwent post-implant CT imaging (1.25 mm isotropic, GE Medical Systems) to reconstruct the DBS leads. With the reconstructed DBS leads, the volume of tissue activation (VTA) in each patient was modeled using the DBS analysis module inside BrainLab Quentry®. Specifically, a 3D reconstruction of VIM, DBS electrode, and the simulated VTAs from the anode and cathode of the DBS leads was visualized. The VTA was then aggregated from the non-TBI DBS patients (excluding TB01) and overlaid them on axial and coronal T2-weighted MRI images (1.6 mm isotropic, TR/TE=9690/91 ms, 3T Siemens Prisma Fit).

### Lesion Segmentation

In TB01, areas of lesion caused by traumatic brain injury were highlighted. Lesion segmentation was performed by board-certified neurosurgeon from axial T2-flair images acquired by a 3T Siemens Prisma Fit Scanner (1.6 mm isotropic, TR/TE=9690/91 ms).

### Statistical Procedures

All statistical comparisons of means presented in the following examples were performed using the bootstrap method, a non-parametric approach which makes no distributional assumptions on the observed data. Instead, bootstrapping uses resampling to construct empirical confidence intervals for quantities of interest. For each comparison, bootstrap samples were constructed by drawing a sample with replacement from observed measurements, while preserving the number of measurements in each condition. 10,000 bootstrap samples were constructed and the difference in means of the resampled data was calculated for each. One or two tailed bootstrapping with alphas of 0.05 (95% confidence interval), 0.01 (99% confidence interval), or 0.001 (99.9% confidence interval) was employed. The null hypothesis of no difference in the mean was rejected if 0 was not included in the confidence interval of the corresponding alpha value. If more than one comparison was being performed at once, a Bonferroni correction was used by dividing the alpha value by the number of pairwise comparisons being performed.

### Example 5

Thalamocortical Projections from the Monkey Motor Thalamus

In order to achieve the aim of increasing the excitability of cortico-spinal neurons within the primary motor cortex, it was sought to identify the optimal stimulation target by localizing a subcortical region that has a high number of direct excitatory projections to the motor cortex and that could be targeted by existing DBS clinical leads and neurosurgical implant strategy. It was posited that the motor thalamus could be this optimal target. In humans, the motor thalamus includes four nuclei that have different preferential projection targets: the ventral anterior (VA), the ventral oral posterior (VOP), the ventral intermediate (VIM), and the ventral caudal nucleus (VC). Thalamocortical fibers originating in the VC project preferentially to the somatosensory cortex, whereas those originating in the VIM/VOP connect to the motor cortex, and those in the VA to the pre-motor cortex. High-resolution diffusion magnetic resonance imaging (MRI) data using high-definition fiber tracking (HDFT) in monkeys (n=3) was acquired and analyzed to confirm whether the monkey motor thalamus had a similar anatomical organization. Analysis was focused on the three nuclei of the monkey motor thalamus: the ventral anterolateral (VAL), the ventral laterolateral (VLL), and the ventral posterolateral (VPL) nuclei. All the likely axonal pathways between these nuclei and the somatosensory, motor, and premotor cortical areas were reconstructed, and the relative strength of these connections was quantified by calculating the volume of thalamocortical projections from each nucleus to each cortical region normalized by the total volume of fibers (FIG. 9A). A clear functional and somatotopic organization of the thalamic nuclei that parallel the organization of the human thalamocortical connections was confirmed, with axonal projections preferentially towards the somatosensory cortex from the posterior motor thalamus (VPL) and preferentially towards motor and pre-motor cortices from the anterior motor thalamus (i.e., VLL and VAL, respectively). Using selectivity detection analysis among the three nuclei, it was found that the VLL nucleus, which corresponds to the human VIM/VOP nucleus, had the greatest selectivity of the projections towards M1 (VPL: 0.67, VLL: 0.89, VAL: 0.33). It was therefore considered the optimal target for the DBS strategy.

### Example 6

### A Robotic Stereotactic Approach to the Motor Thalamus in Monkeys

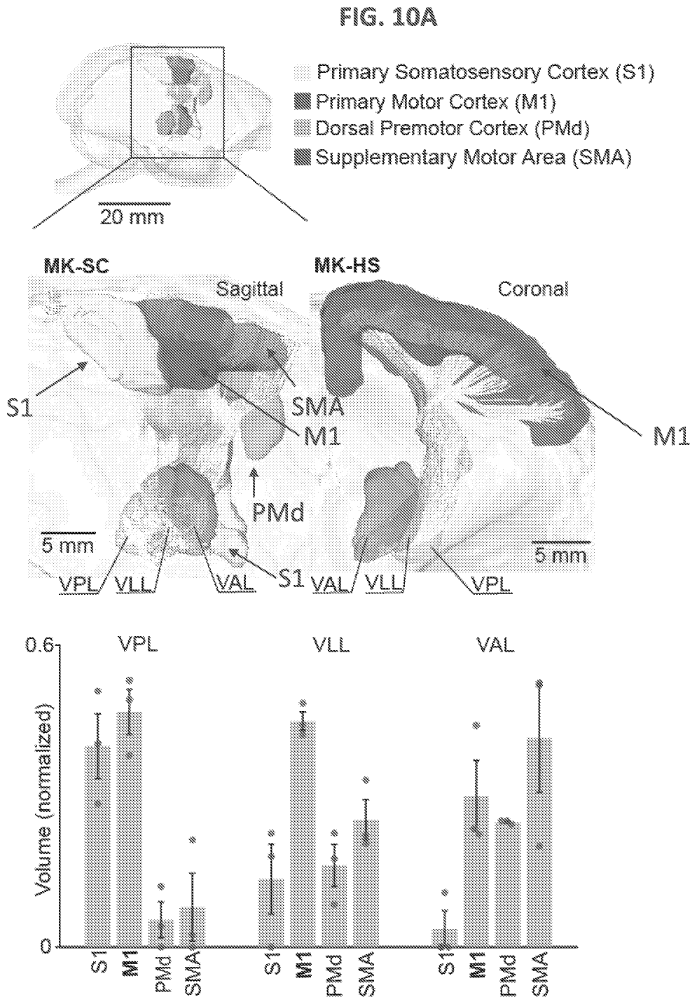

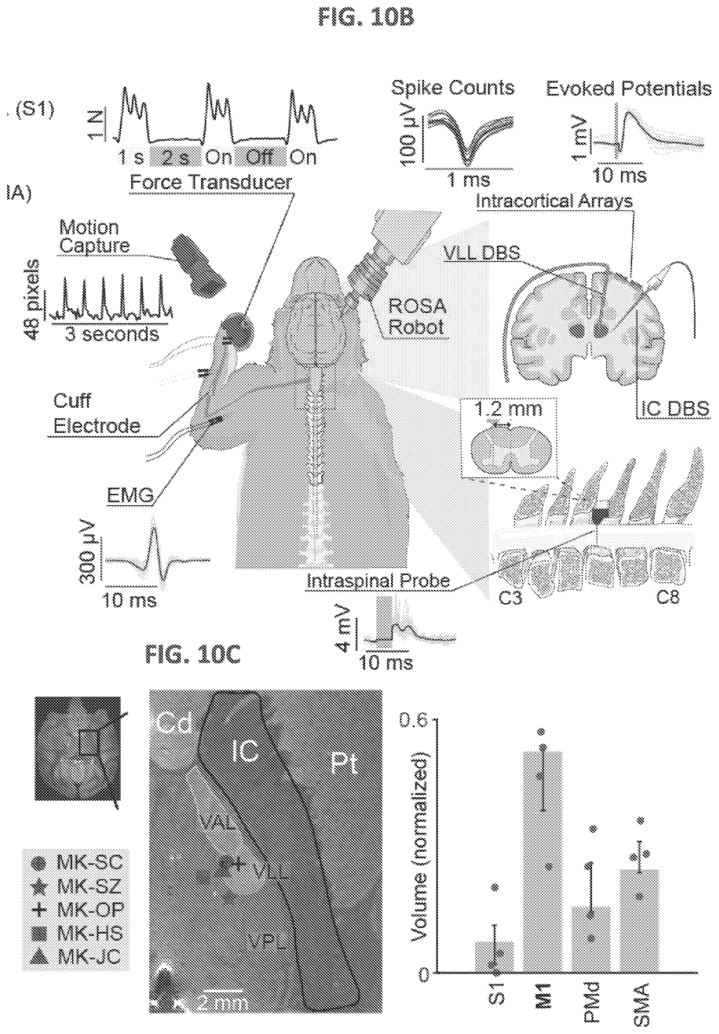

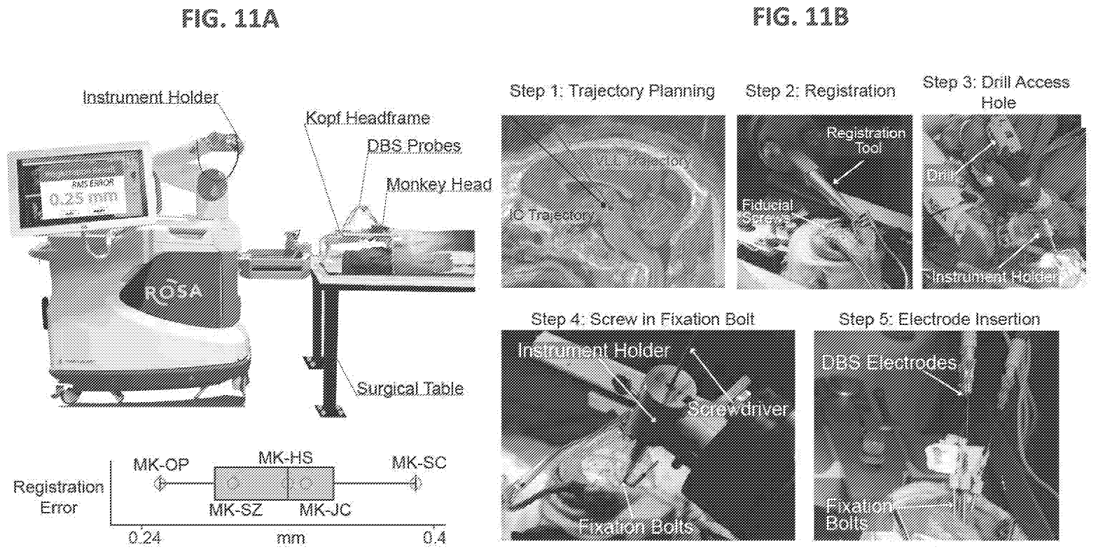

To enable a highly precise implantation of the stimulating electrodes in the VLL in monkeys, a medical-grade MRI-guided robotic stereotactic device was repurposed (FIG. 10B). This allowed implantation procedures similar to those that could be surgically applied in humans thus facilitating immediate translation of results to patients. A ROSA ONE® Robot Assistance Platform (ROSA robot, Zimmer Biomet, Warsaw, Indiana, USA) was used to place a human-grade electrode (Microdeep® SEEG Electrodes, DIXI Medical, Marchaux-Chaudefontaine, France) within the VLL nucleus in anesthetized monkeys (n=5) (FIG. 11). High-definition anatomical MRIs were obtained to plan a single trajectory to the VLL that avoided the sensory and motor cortices. MRIs were then fused to computer tomography (CT) scans obtained after implantation of five fiducial titanium skull screws to use as reference points for co-registration of the robot during surgery. Co-registration errors were small (MK-SC: 0.39 mm, MK-SZ: 0.32 mm, MK-OP: 0.25 mm, MK-HS=0.29 mm, MK-JC=0.33 mm), thus ensuring highly precise implantation. These errors were consistent with previous report (FIG. 11) and similar to the errors observed in human DBS implantations (0.27±0.07 mm). At the end of the experimental procedures, a small thermocoagulation lesion was created at the electrode tip to allow post-mortem visualization of the implant location. High-resolution post-mortem structural MRI confirmed the accurate location of the DBS electrode within the VLL (FIG. 10C). Additionally, HDFT was used between the electrode implantation region and the somatosensory, motor, and pre-motor cortical areas and it was confirmed that the largest volume of fibers within the stimulation field projected to M1 (on average 55% of the fibers) (FIG. 10C).

### Example 7

### Targeted Deep Brain Stimulation of the VLL Specifically Excites the Motor Cortex

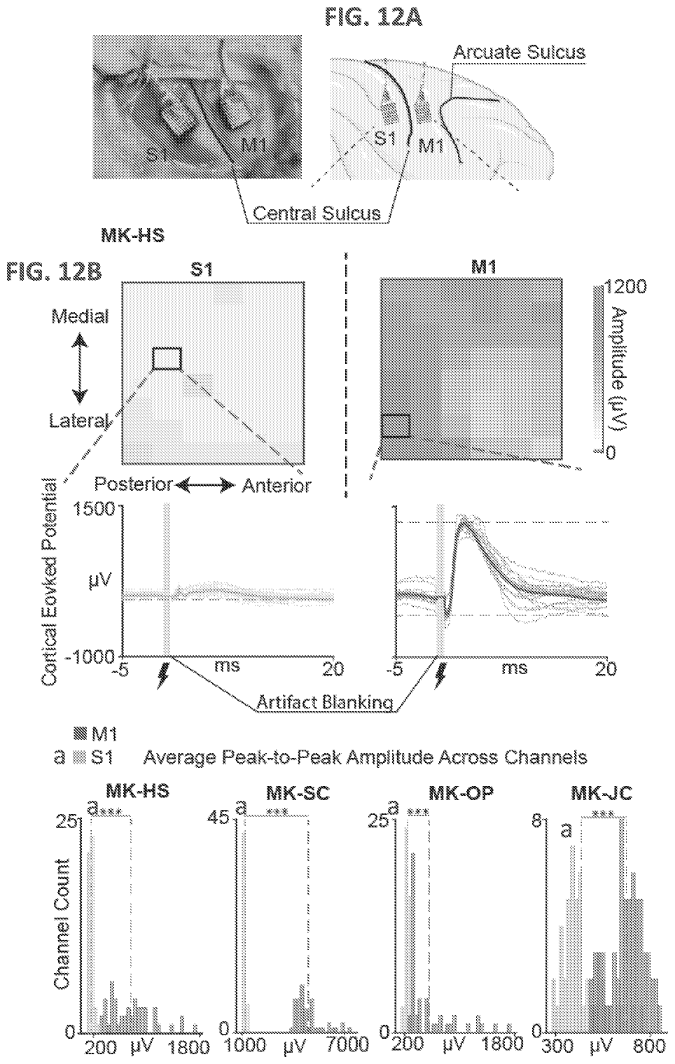

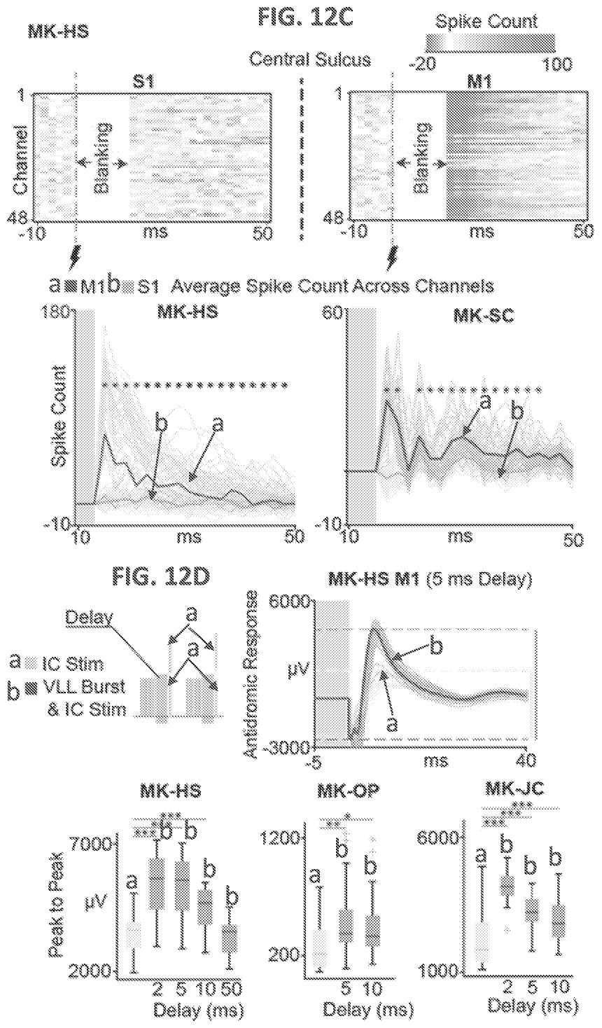

Thalamocortical projections, as identified with our HDFT analysis, are glutamatergic. Therefore, it was hypothesized that stimulation of the VLL would excite the motor cortex. To test this hypothesis, two 48-channels intracortical arrays (Utah arrays), in addition to the DBS electrode within the VLL nucleus, were implanted in the primary motor and somatosensory cortices, respectively (FIG. 10B). In n=4 anesthetized monkeys, continuous stimulation of the VLL at 10 Hz was applied (see Table 2 for details on stimulation parameters) while recording local field potentials and multi-unit neuronal activity from these cortical areas (FIG. 12A). VLL stimulation triggered averages of evoked potentials in M1 and S1 electrodes were analyzed, and clear cortical evoked potentials with peak latencies at 5-10 ms post-stimulation were observed (see example FIG. 12B) confirming a direct monosynaptic pathway between the VLL and cortex. Analysis of peak to peak amplitude across the entire arrays showed significantly larger responses in M1 as compared to S1 in all monkeys (see histograms FIG. 12B), confirming preferential projections of VLL axons towards M1. Interestingly, these responses were larger along the pre-central gyrus where the hand and arm representation in M1 are located (see heat maps FIG. 12B). Stimulation-evoked spike counts corroborated these findings showing an increase in multi- and single-units firing rates post-stimulation (FIG. 12C). This increase was significantly stronger in M1 as compared to S1.

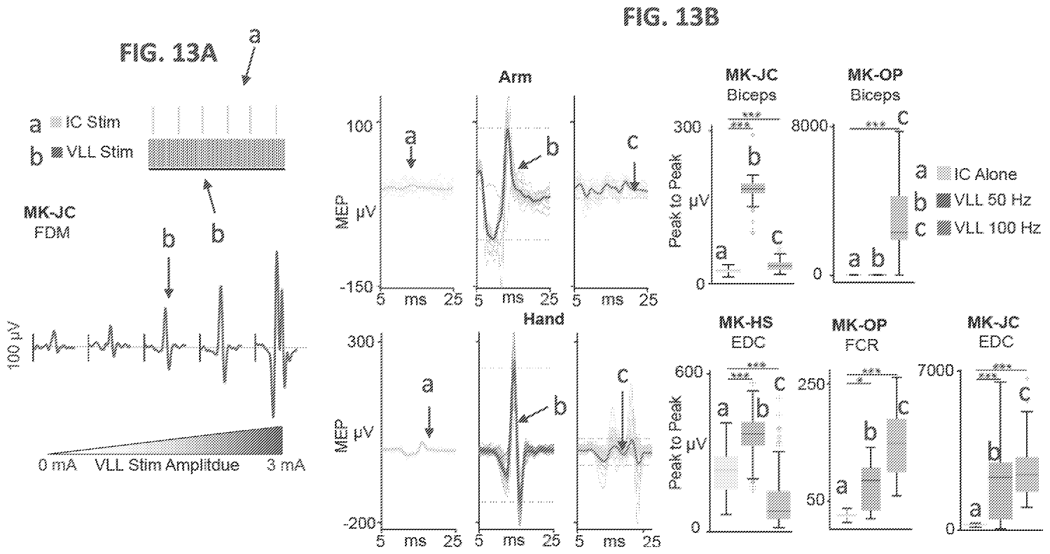

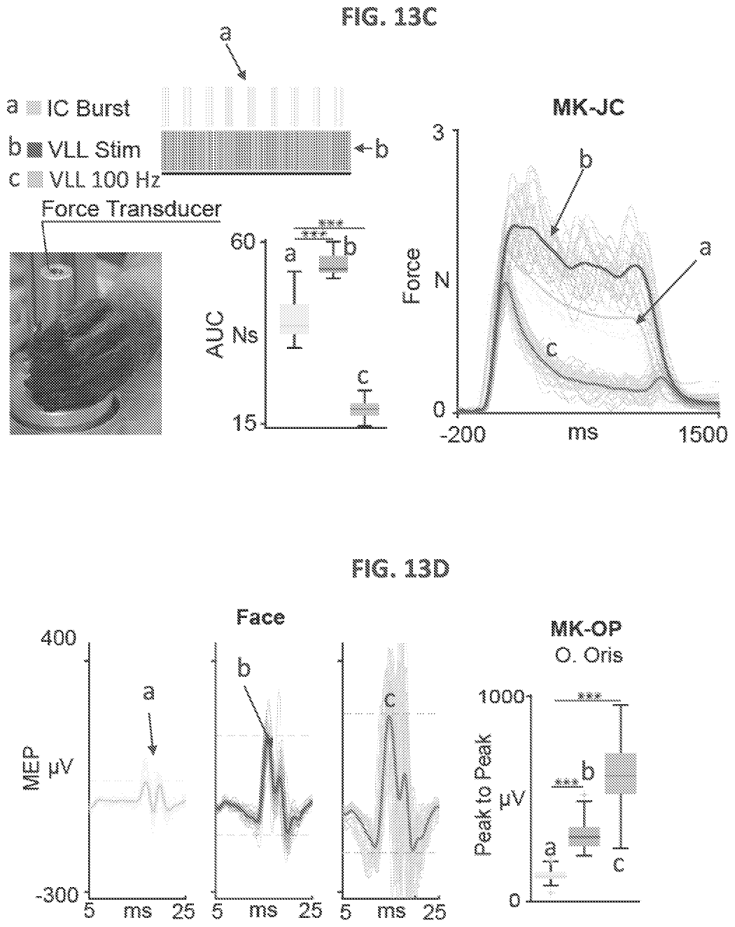

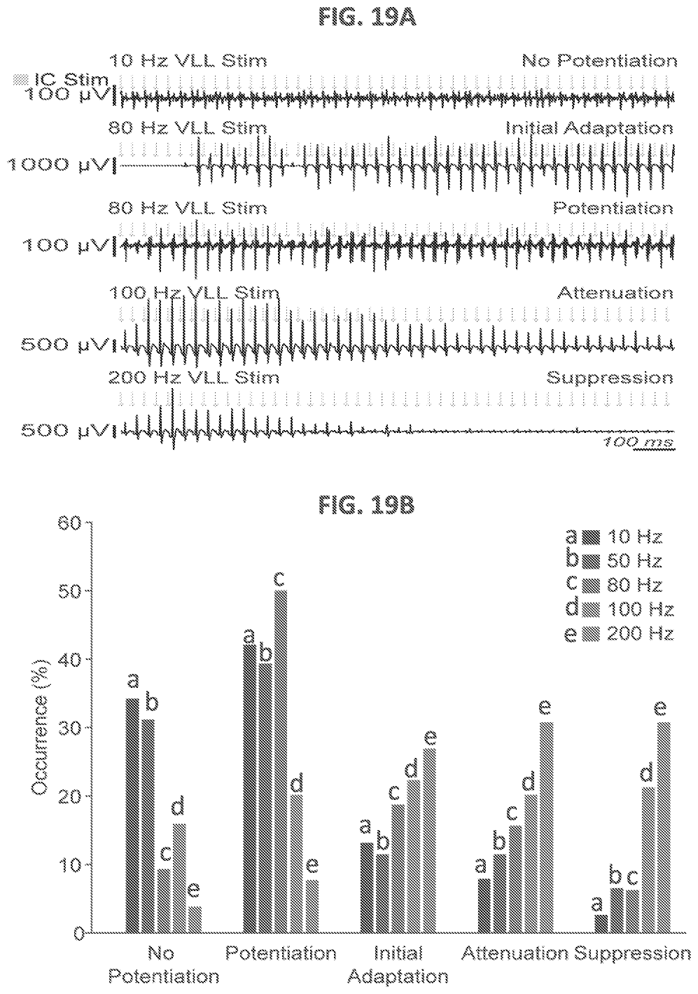

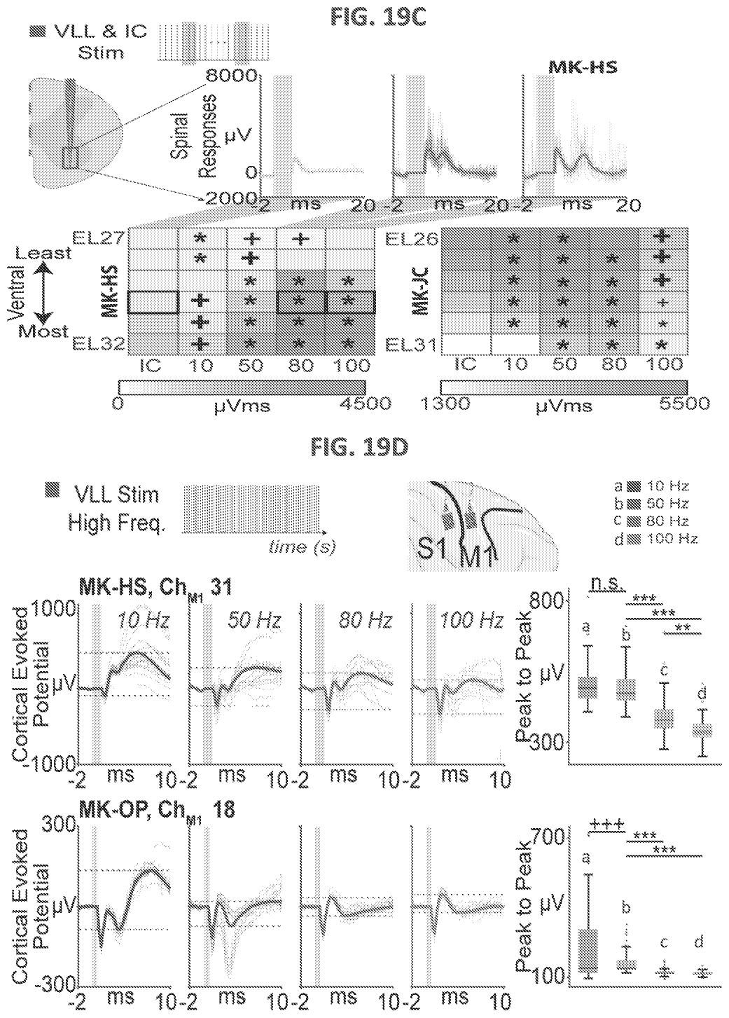

Cortical evoked potentials elicited from VLL stimulation should be indicative of increased excitability of cortico-spinal neurons. If this is true, the amplitude of antidromic neural responses elicited in cortical-spinal axons from stimulation of the CST and recorded in M1 should be larger when VLL stimulation is active because of decreased cortical thresholds in these neurons. To test this property, a stimulating electrode was implanted into the posterior limb of the internal capsule (IC) (FIG. 10B), which contains the cortical-spinal axons originating in the motor cortex and projecting to the spinal cord. The hand representation of the CST was targeted. Direct stimulation of these axons creates both orthodromic action potentials towards the spinal motoneurons (FIG. 19C) and antidromic action potentials toward the cell body of the pyramidal neurons in M1, which could be recorded through the intracortical electrodes (FIG. 12D). Implantation within the CST passing through the IC was confirmed, observing MEPs of upper arm and hand muscles (10-15 ms post-stimulation, FIG. 13B) and antidromic evoked potentials in M1 at shorter latencies (5 ms post-stimulation, FIG. 12D). Single pulse stimulation was then delivered to the IC conditioned by a 100 ms burst at 100 Hz to the VLL nucleus at different delays (2-50 ms). The peak to peak amplitude of the antidromic neural responses in M1 was found to be significantly higher when a burst of VLL stimulation conditioned IC stimulation (n=3) (FIG. 12D) confirming that VLL stimulation increases excitability of cortico-spinal neurons. At delays higher than 10 ms, the antidromic potentials returned to amplitudes similar to those of IC stimulation alone.

Overall, these results demonstrate that DBS of the VLL nucleus increases the excitability of cortico-spinal neurons specifically within M1.

### Example 8

### Motor Thalamus Stimulation Potentiates Upper-Limb Motor Outputs

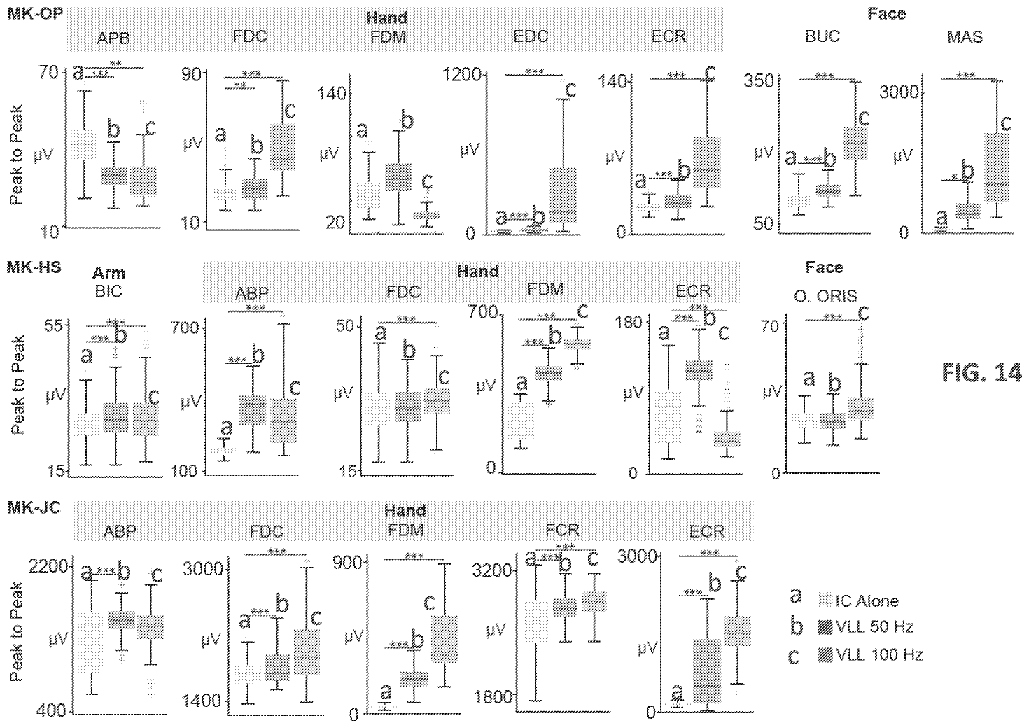

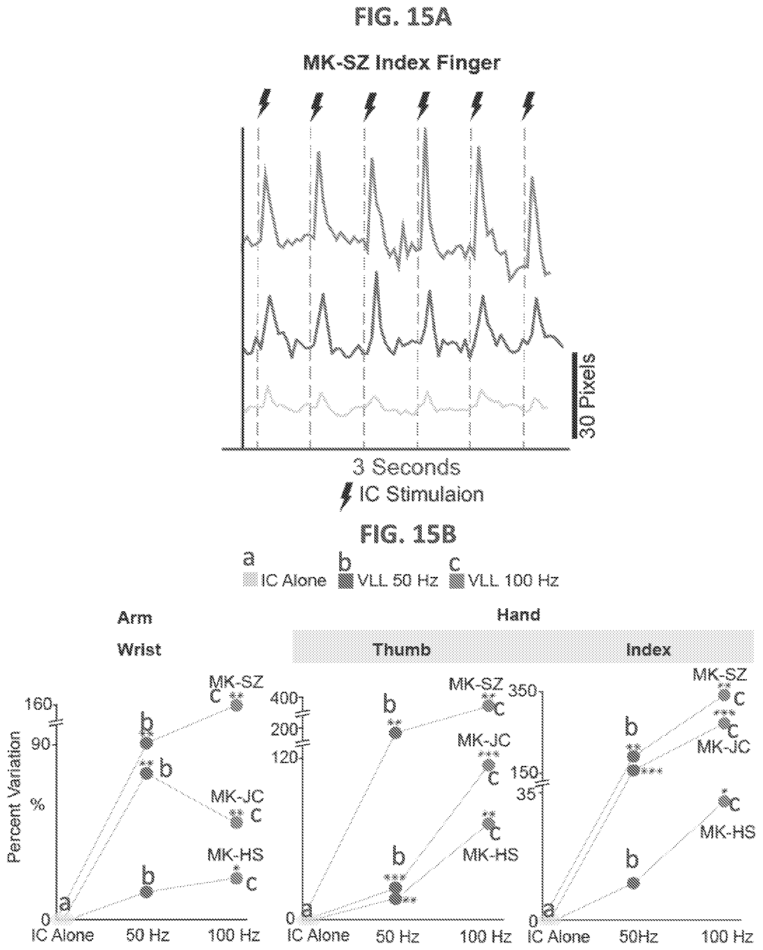

It was hypothesized that that the enhanced cortical excitability observed in our monkeys would lead to amplified MEPs recorded in arm, hand, and face muscles. Stimulation of the IC at 2 Hz was paired with continuous stimulation of VLL at 50 Hz or 100 Hz (FIG. 13A). Stimulation of the IC at 2 Hz generated MEP responses in hand, arm, and face muscles as well as movements of the arm and face (FIG. 13B) at short latencies (on average 10-15 ms) suggesting a monosynaptic activation of motoneurons. When 2 Hz IC stimulation was paired with continuous stimulation of the VLL at 50 Hz and 100 Hz, peak to peak amplitude of both MEPs and movements of the arm (n=2) and hand (n=3) were immediately and significantly increased (FIGS. 13B, 14, and 15). Interestingly, MEP peak to peak amplitudes increased proportionally to VLL stimulation amplitude (FIG. 13A). In one animal, VLL stimulation also increased face muscles MEPs (FIG. 13D). While the frequency at which maximal increases were obtained varied across animals and muscles (50 Hz or 100 Hz), MEPs and movement were augmented in all tested monkeys (FIGS. 14 and 15). More specifically, VLL stimulation at 50 Hz most often increased MEP amplitudes in the upper-limb and face; whereas, stimulation at 100 Hz would produce either greater amplification or in some instances a suppression of MEPs (FIG. 13B, 19A).

Finally, in order to demonstrate that VLL stimulation could enhance induced functional movements, bursts of IC stimulation were delivered at about 50 Hz which induced a grasping motion producing measurable isometric forces (FIG. 13C). The functional IC stimulation was paired with VLL stimulation at 50 Hz or 100 Hz. VLL stimulation at 50 Hz, but not at 100 Hz, immediately and significantly increased the grip force as compared to no VLL stimulation (n=1) (FIG. 13C).

In summary, targeted motor thalamus stimulation increased upper-limb motor output as measured by the amplitude of arm and hand muscle MEPs and movement kinematics, and stimulation-induced grip forces.

### Example 9

### Motor Output Potentiation Occurs Through Orthodromic Thalamocortical Pathways

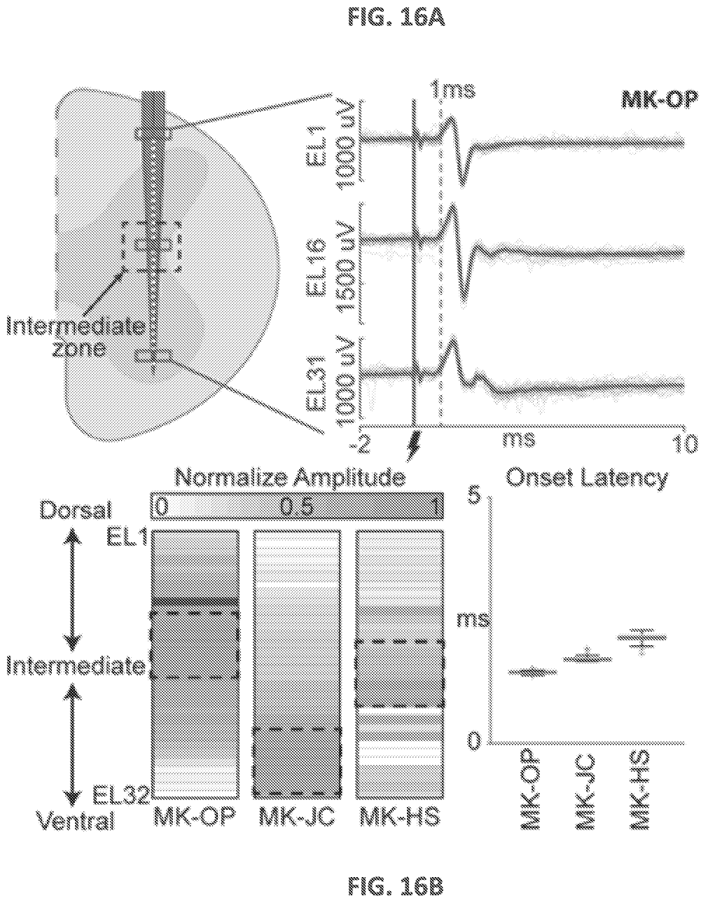

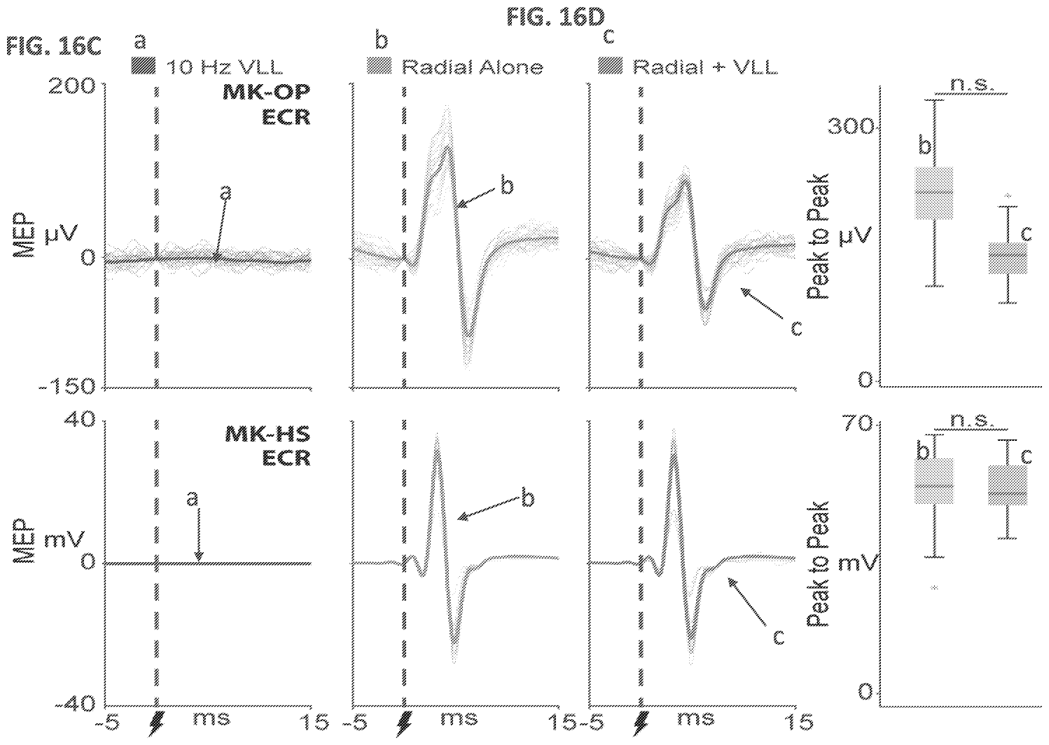

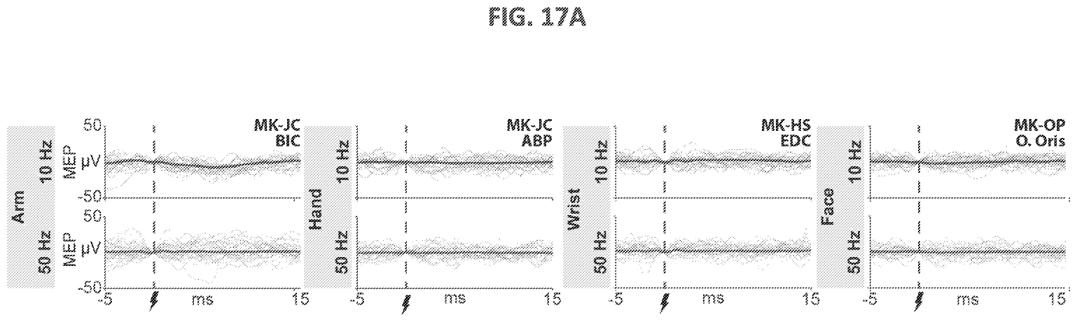

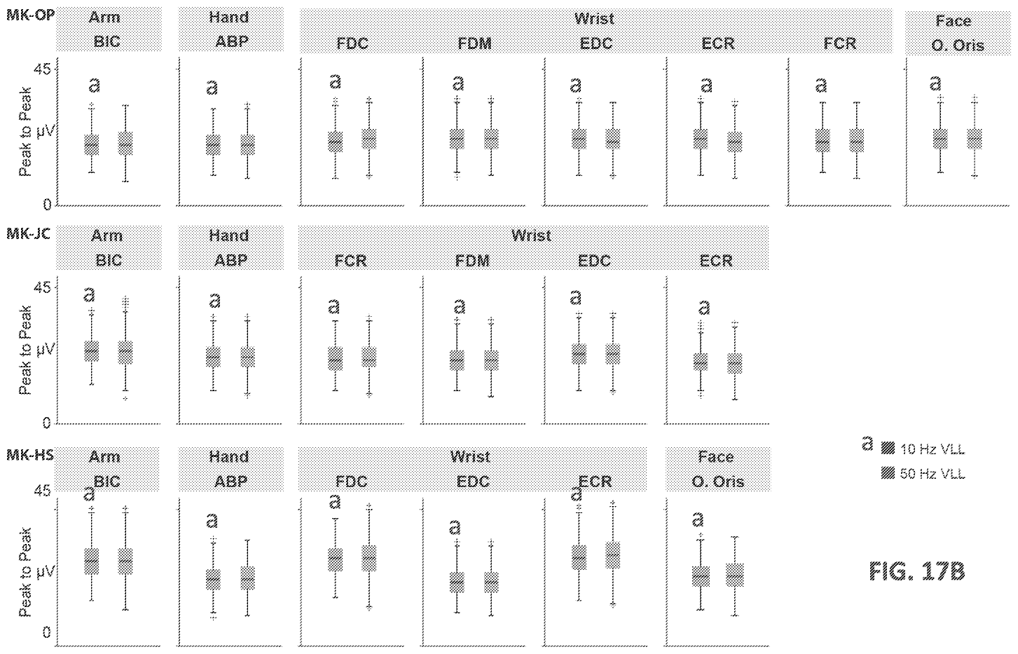

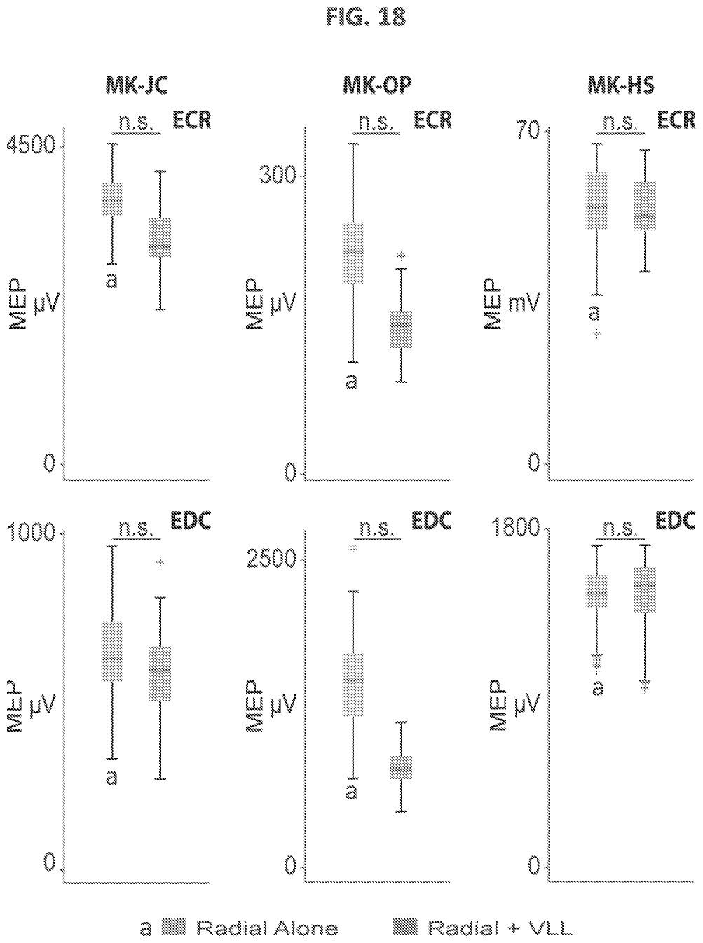

This example illustrates that the enhanced motor outputs were the result of an increased excitability of the motor cortex and did not result instead from inadvertent activation of descending excitatory tracts from current spread. It was reasoned that if the observed MEP potentiation was occurring within the spinal cord as a result of inadvertent descending action potentials in other excitatory tracts, then peripheral inputs to spinal circuits, such as H-reflexes, would also be modulated by VLL stimulation. To control for this, a multi-channel linear probe (NeuroNexus) was implanted at the C6-C7 spinal level with dorsal-ventral orientation (FIG. 10B). Consistent volleys were observed in the spinal cord in the first 3 ms following VLL stimulation (1.44 ms, 1.73, and 2.13 ms for Mk-OP, Mk-JC, and Mk-HS, FIG. 16A) with largest peak to peak amplitude in the intermediate-ventral spinal cord gray zone (FIG. 16B). These evoked potentials could either represent antidromic recruitment of ascending pathways such as spino-thalamic axons, or orthodromic activation of descending excitatory pathways. However, there were no MEPs in either the upper-limb or facial muscles when stimulating the VLL nucleus alone FIG. 16C, FIG. 17) suggesting that the observed spinal responses were not caused by inadvertent stimulation of the CST. Moreover, the frequency of the movements when the VLL and the IC were simultaneously stimulated was at 2 Hz (i.e., same frequency of IC stimulation) and not at the frequencies of VLL stimulation (50 or 100 Hz) (FIG. 15), further demonstrating that the increase in MEPs and movement kinematic was not induced by current spread from VLL to the CST. It was still possible that the observed spinal responses and the enhanced movements could be carried by other descending tracts that do not have direct spinal motor neuron connections or generate MEPs but that are able to excite the spinal circuits and facilitate movement. However, when continuous VLL stimulation at 50 Hz was paired with stimulation of the radial nerve no significant increase of reflex-mediated responses was observed (FIGS. 16D and 18). These results demonstrate that 1) the observed volleys in the spine are likely the result of antidromic activation of ascending pathways such as spino-thalamic axons; and 2) the observed potentiation of MEPs (FIG. 13) is not occurring inside the spinal cord as a result of current spread to descending axons, but is, instead, a trans-cortically mediated effect.

### Example 10

### Efficacy of VLL Stimulation in Promoting Motor Augmentation is Frequency Dependent

The results of this disclosure suggest that the potentiation of descending CST activity is mediated by the orthodromic recruitment of cortico-spinal neurons in M1 via thalamocortical excitatory synaptic projections. Excitatory synaptic transmission is known to be affected by stimulation frequency via mechanisms such as presynaptic inhibition or homosynaptic neurotransmitter suppression. Accordingly, the effect of multiple frequencies on the efficacy of motor potentiation was explored. IC stimulation at 2 Hz was delivered with continuous VLL stimulation at 10, 50, 80, 100, and 200 Hz. The MEP response to the individual IC stimulation pulses was analyzed over time; a variety of modulation patterns were observed (FIG. 19A). These patterns were classified into five distinct categories: (1) no potentiation, as responses were comparable to IC stimulation alone, (2) initial adaptation, as responses were amplified upon activation of VLL stimulation but only after an initial transient period of 5-20 responses, (3) sustained potentiation, as responses were potentiated, (4) attenuation, as the response amplitudes progressively decreased compared to the initial responses, and (5) suppression, as the responses were completely suppressed after a few pulses. Generally, the absence of potentiation of the IC stimulation responses was characteristic of low frequencies (10 Hz), whereas stimulation frequencies between 50 Hz and 80 Hz consistently increased the MEP amplitudes with sustained outputs (FIG. 19B). Stimulation at 100 Hz, instead, resulted in potentiation and initial adaptation of responses, but effects varied across muscles with some instances of MEP suppression. Finally, attenuation and suppression of responses were more likely to happen at higher frequencies and particularly at 200 Hz. Therefore, we identified the 50-100 Hz range as optimal to achieve sustained potentiation of MEPs.

The neural correlates of these effects were explored with intra-cortical and intra-spinal neural recordings. Consistent with the EMG recordings, intra-spinal neural responses elicited by IC stimulation pulses in the ventral zone, where spinal motoneurons are located, had significantly larger peak to peak amplitudes when the VLL nucleus was stimulated at 50 or 80 Hz (FIG. 19C). Spinal responses were, instead, suppressed at higher frequencies. Similarly, cortical evoked potentials in M1 when stimulating the VLL alone showed stronger peak to peak amplitudes with frequencies of stimulation in the 50-80 Hz range as compared to 100 Hz (FIG. 19D). These combined results demonstrate that the effects observed in the MEP and spinal cord responses were a consequence of frequency dependent excitation of the motor cortex. This frequency dependent characteristic of excitation could be either the result of presynaptic inhibition or homosynaptic neurotransmitter suppression at the level of the cortico-spinal neurons.

In summary, these results confirm a frequency dependent effect that is consistent with motor cortical potentiation of thalamic afferents to M1 cortex via excitatory synaptic inputs. From these findings, it was identified that the optimal stimulation frequencies to increase muscle outputs are in the 50-100 Hz range, notably lower than clinical DBS stimulation frequencies (˜130 Hz).

### Example 11

Potentiation of Motor Outputs Persists after CST Lesions

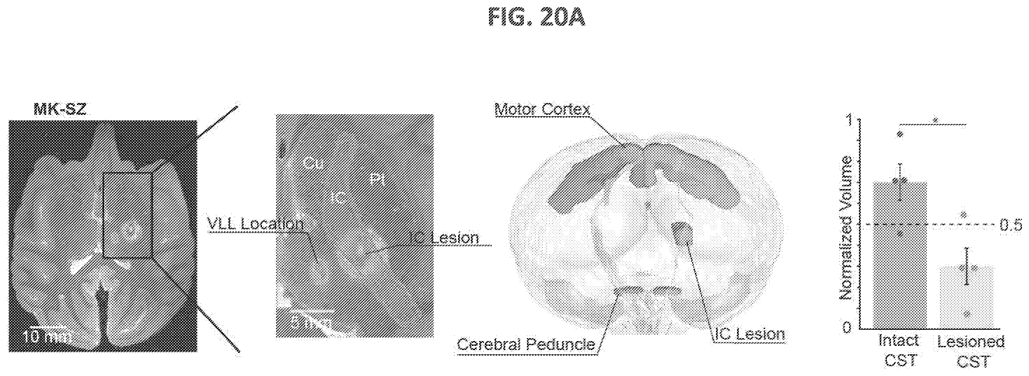

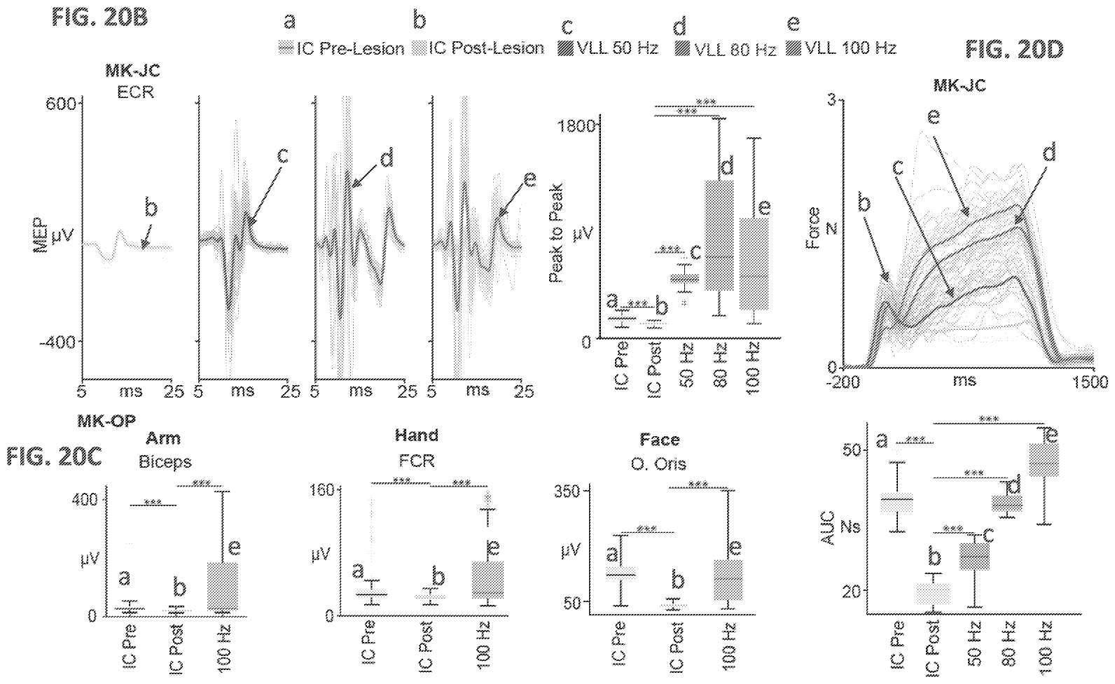

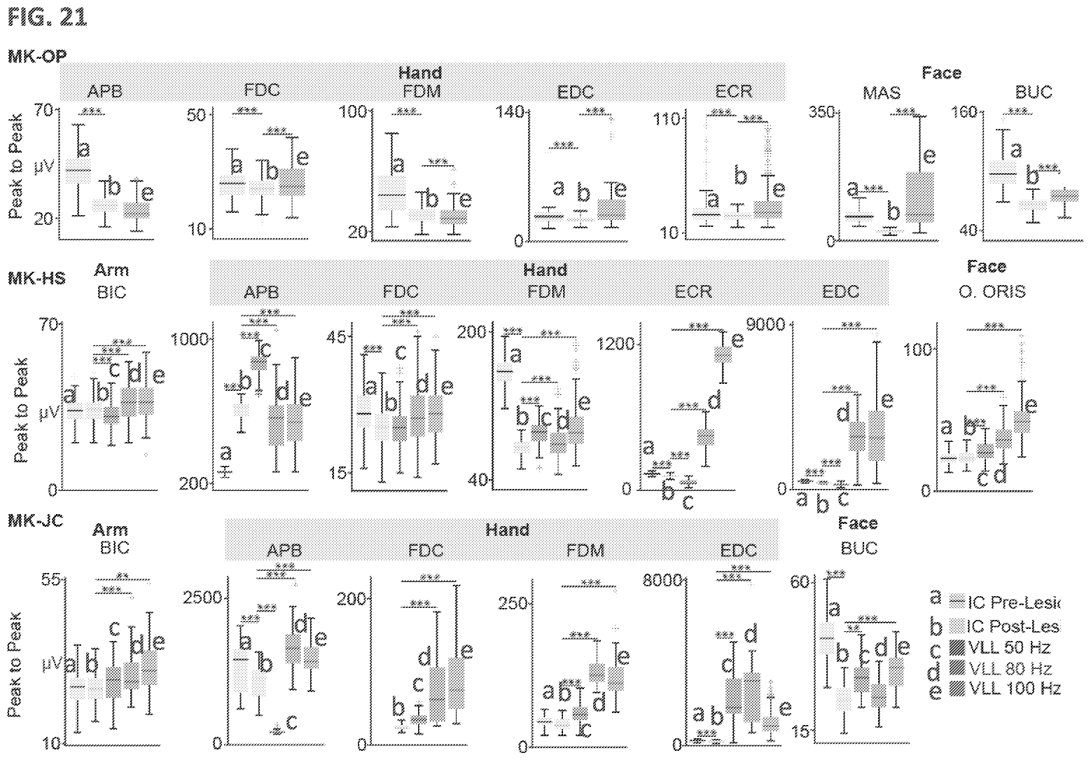

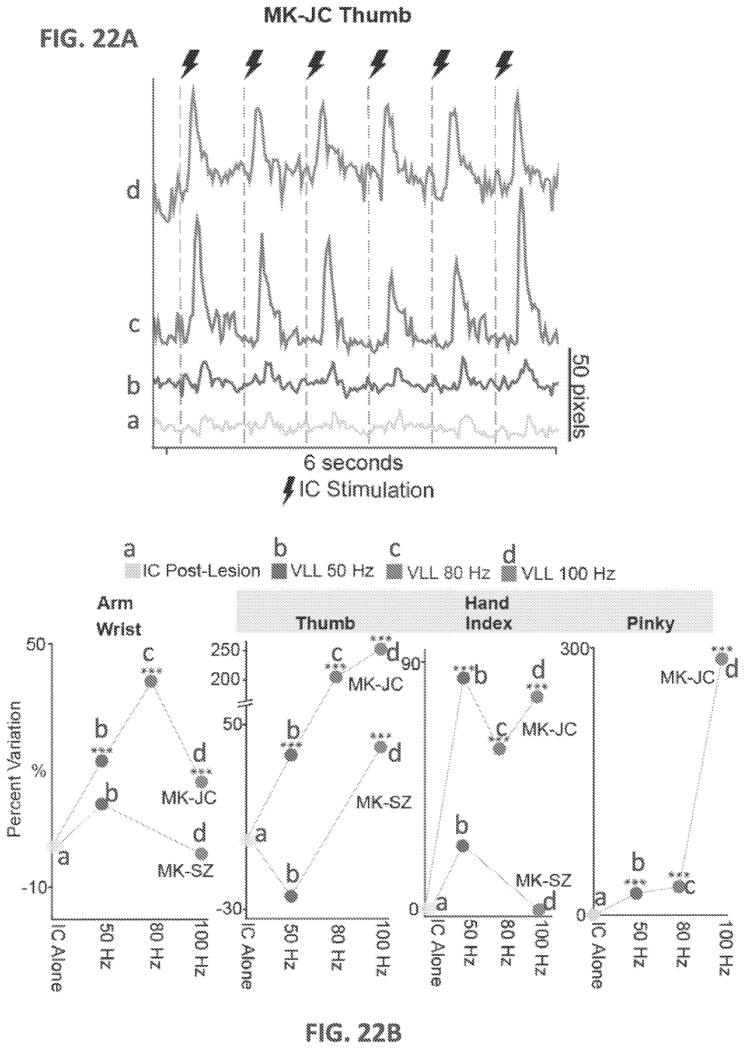

To demonstrate the potential clinical relevance of this approach, it was verified that electrical stimulation of the VLL nucleus would still augment motor outputs after a lesion of the CST. A partial lesion of the IC would reduce the number of CST fibers projecting to the cervical spinal cord, leading to motor deficits similar to those observed in stroke and TBI patients. To create such a lesion, a second depth electrode was implanted in the posterior limb of the IC, approximately 2 cm ventral from the IC stimulating electrode. A controlled and reproducible lesion of the CST fibers was then generated by performing thermo-frequency ablations (as described in Vinas et al. Stereotactic and functional neurosurgery (1992) 58:121-33) through the ventral electrode (FIG. 20A). Reduced MEP amplitudes were observed when stimulating the dorsal IC electrode after the ventral lesion had been made (FIGS. 20B and 20C). Post-mortem HDFT confirmed that the lesion consistently and significantly reduced the volume of CST fibers compared to the intact hemisphere in all tested monkeys (70% of reduction) (n=4) (FIG. 20A). After the IC lesion, the smaller MEPs recorded from the arm, hand and face muscles were significantly increased when the VLL nucleus was simultaneously stimulated at 50 and 100 Hz (FIGS. 20B, 20C, 21, and 22). MEP amplitudes when VLL stimulation was ON were comparable to pre-lesional MEP amplitudes in the same muscles. Importantly, also the grip force elicited by IC stimulation bursts significantly increased with continuous VLL stimulation at 50 Hz and an even greater increase occurred with VLL stimulation at 80 and 100 Hz (FIG. 20D).

Overall, these results demonstrate that VLL stimulation can potentiate motor output even in the presence of hemiparesis caused by lesions located within the CST.

### Example 12

### Motor Thalamus DBS Potentiates Motor Output in Humans

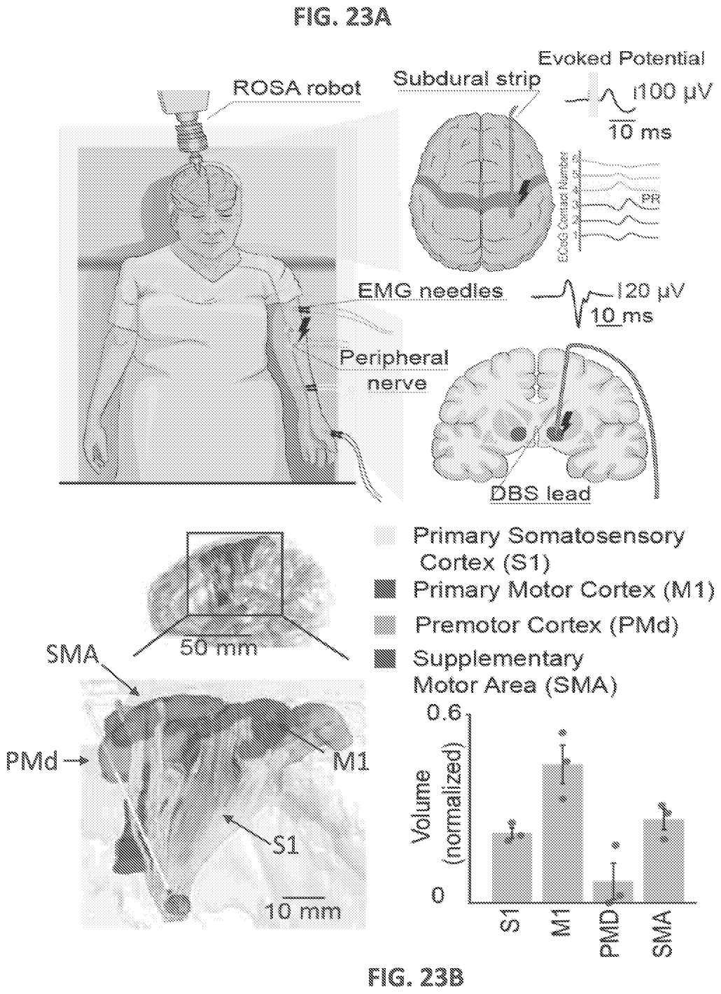

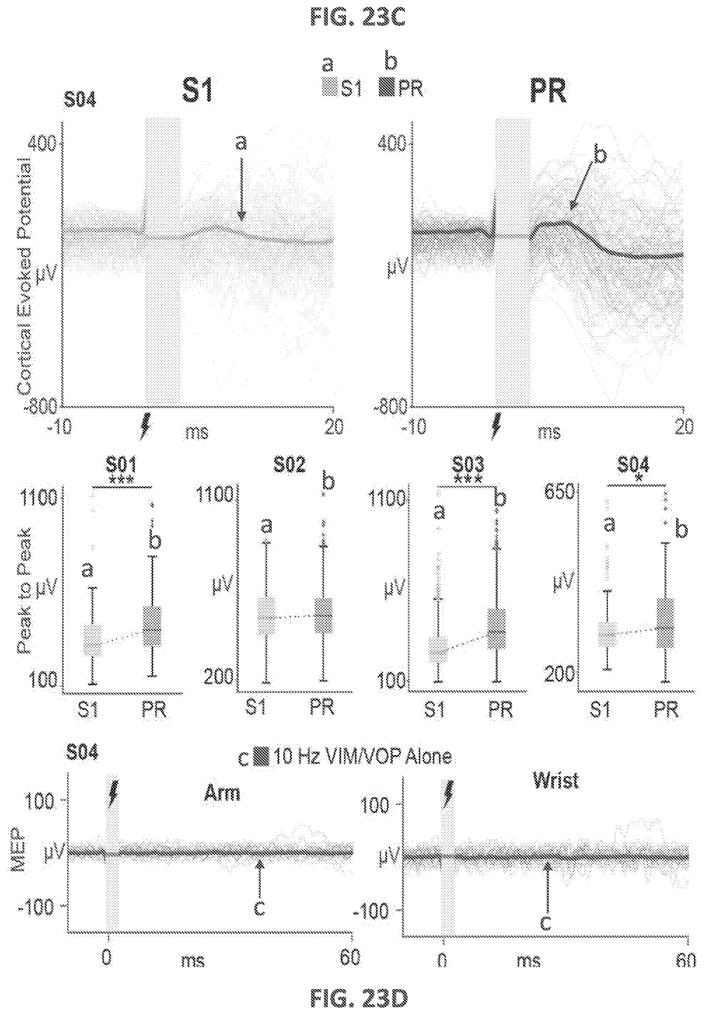

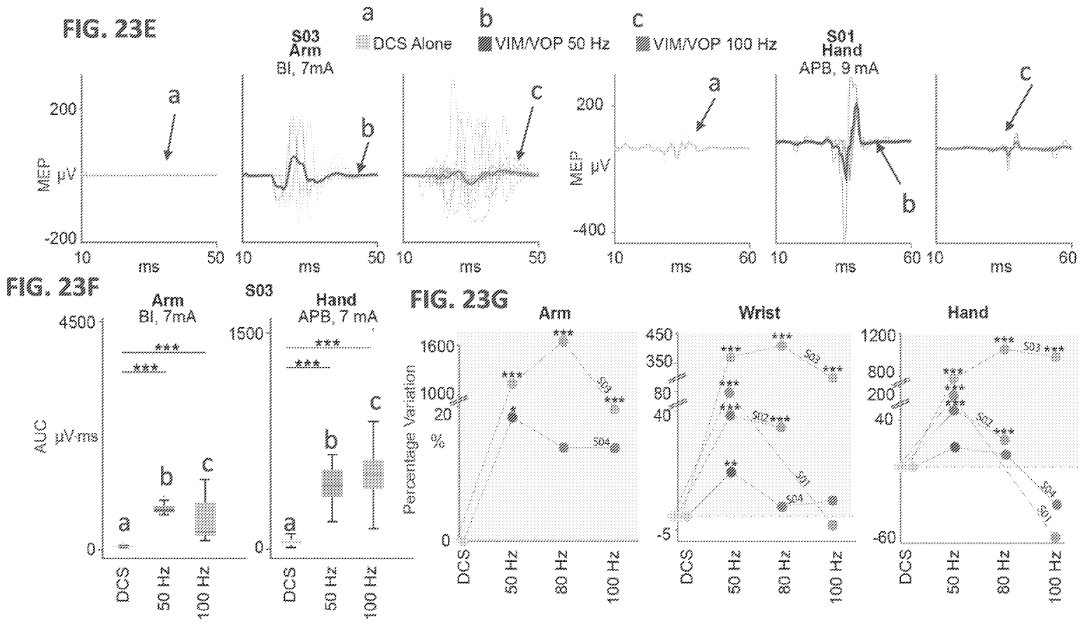

To demonstrate the translational capabilities of these DBS methods intraoperative neurophysiological experiments were performed in human subjects (n=4, 2 male, 2 female) after obtaining informed written consent. The subjects underwent surgical DBS implantation of the motor thalamus for the treatment of Essential Tremor (ET) (FIG. 23A). Experiments were designed to closely mimic the animal study protocols. The human motor thalamus nuclei comparable to the VLL nucleus are the ventralis intermediate (VIM) and the ventralis oralis posterior (VOP). HDFT analysis confirmed preferential anatomical projections from the VIM/VOP to M1 (FIG. 23B). The connectivity of these pathways was tested using evoked potential methods. For this, a 6-channel subdural strip electrode (Adtech, Oak Creek, WI, USA) was implanted over the upper-limb representation of the primary motor and somatosensory cortices. The validated clinical technique of somatosensory evoked potential (SSEP) phase reversal (PR) mapping (described in Cedzich et al., Neurosurgery (1996) 962-70) was used to locate both the hand representation of S1 (largest amplitude N20/P30 cortical SSEP) and the approximate location of the central sulcus, where the polarity of the SSEP reverses in order to confirm electrode placement (FIG. 23A). The upper extremity representation of M1 at the precentral gyrus was mapped by direct cortical stimulation (DCS) of the electrode contacts that were anterior to the SSEP PR. MEPs were recorded from six contralateral upper extremity muscles (Deltoid, Biceps, Triceps, Flexor carpi, Extensor carpi and APB). The strip electrode was adjusted so that the lowest threshold (˜6-12 mA) MEPs were recorded from at least three muscles, and the electrode was fixed in place.

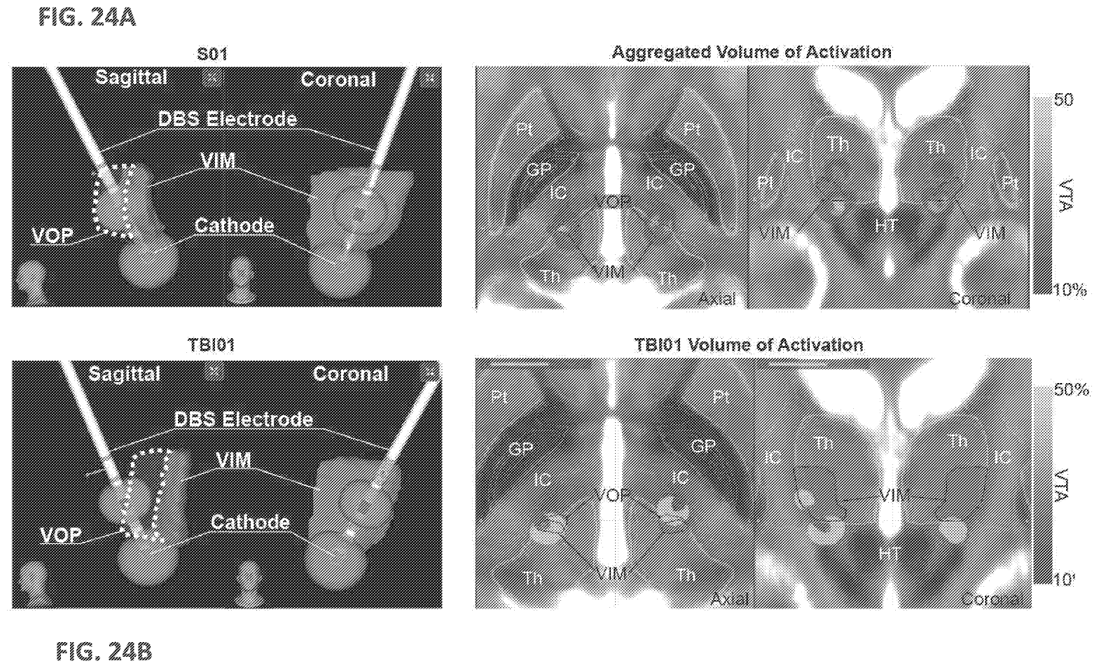

The projections from VIM/VOP to S1 and M1 upper extremity representations were examined by recording cortical evoked potentials at each of the 6 electrode contacts in response to low frequency (2 or 10 Hz) stimulation of the VIM/VOP DBS electrode (Boston Scientific) implanted in the same hemisphere at the AC/PC plane (X: 12 mm lateral to the AC/PC line, Y: 6 mm anterior to PC, Z: 0 mm to the AC/PC horizontal plane, FIG. 24). Cortical evoked potentials, occurring within 20 ms after the stimulus, were recorded at the M1 contacts (rostral to the PR location) (FIG. 23C). In all subjects but one, the peak to peak amplitude of these evoked responses was significantly larger for precentral contacts than for postcentral contacts. This supports orthodromic synaptic transmission through thalamocortical projections from VIM/VOP to the M1 hand/arm representation as was observed in monkeys during stimulation of the VLL.

Next, DCS MEPs from up to six contralateral upper extremity muscles were recorded with and without paired VIM/VOP DBS stimulation at 50, 80, or 100 Hz. Similar to the monkey experiments, a consistent increase in the DCS MEP amplitudes across arm, wrist and hand muscles with VIM/VOP stimulation at 50 Hz was observed compared to DCS alone.

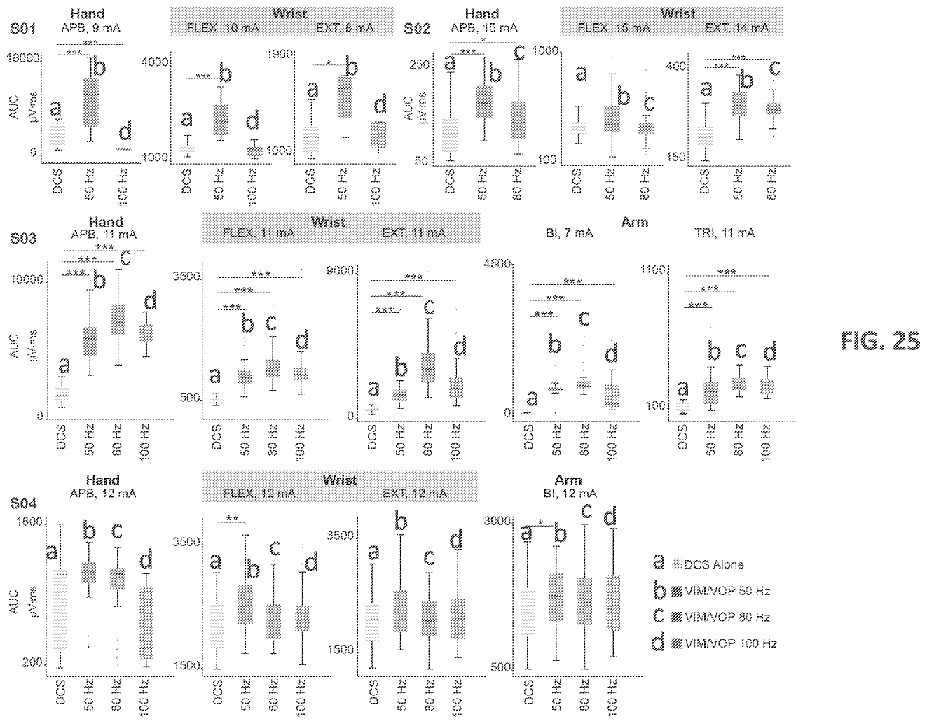

A more variable behavior was found with VIM/VOP stimulation at higher frequencies (100 Hz, FIG. 23F). Indeed, when quantifying the effect of VIM/VOP stimulation across stimulation frequencies and muscles, it was found that 50 Hz DBS consistently enhanced muscular responses in a statistically significant way, whereas higher frequencies such as 80 and 100 Hz might further amplify or suppress these responses (FIGS. 23G and 25). Finally, it was confirmed that, similar to the monkey experiments, electrical stimulation of the VIM/VOP alone did not produce motor evoked potentials in the arm, wrist, nor hand muscles (FIG. 23D).

These results observed in human subjects were equivalent to those obtained in monkeys, demonstrating the translational potential of the disclosed approach and associated mechanisms of action. Specifically, electrical stimulation of the motor thalamus enhanced motor cortex excitability and consequently potentiated motor output via the CST in human subjects.

### Example 13

Motor Thalamus DBS Increases Motor Output after TBI

This example illustrates application of motor Thalamus DBS to a patient who had suffered a severe traumatic brain injury as a consequence of a motor vehicle accident (FIG. 26A). The severe trauma resulted in bilateral diffuse axonal injury to the CST, cerebral peduncles, and pons. Consequently, TBI01 suffered from hemiparesis and tremor of the right and left upper extremities. Functionally, TBI01 required maximal assistance for eating, grooming, bathing, and dressing. With the clinical goal of treating the post-traumatic tremor, TBI01 was implanted with bilateral DBS electrodes in the VIM/VOP using standard stereotactic coordinates (X: 12 mm lateral to the AC/PC line, Y: 6 mm anterior to PC, Z: 0 mm to the AC/PC horizontal plane, FIG. 24). As was the case in the non-TB subjects, a 6-contact subdural strip electrode was implanted over S1 and M1 cortex of the left hemisphere (i.e., greater TBI damage). DCS MEPs were consistently recorded from the APB and flexor muscles of the hand and wrist (FIG. 26C, left panels). Consistent with the non-TBI subjects, stimulation of the VIM/VOP nucleus resulted in larger amplitude cortical evoked potentials over motor cortical areas as compared to somatosensory cortex (FIG. 26B). Then DCS of M1 was paired with DBS of the motor thalamus to assess the effect of VIM/VOP stimulation on the hemiparetic arm. Again, DCS MEPs were significantly larger with concurrent stimulation of the motor thalamus at 50 Hz and modulated in a frequency-dependent manner (FIG. 26C, right panels). 100 Hz stimulation resulted in suppression of MEPs, demonstrating the consistency of our findings across monkeys, non-TBI subjects, and a patient suffering from a chronic (>6 months) CST lesion.

### Example 14

### Motor Thalamus Stimulation Improves Voluntary Force Control

To assess whether DBS of the motor thalamus achieves functional improvements for patients with chronic motor deficits, the effect of VIM/VOP DBS in TBI01 during a functional task was tested over several testing sessions using the chronically (>5 months) implanted DBS system. After informed consent was obtained, TBI01 performed an isometric roadway test to measure his voluntary force control with and without bilateral VIM/VOP stimulation at 55 Hz. The task involved matching grip force to a time series of thresholds, gradually increasing, sustaining, and decreasing the force between set percentages of maximum voluntary force levels that were established when DBS was OFF (FIG. 26D). This experiment mimics those performed in anesthetized monkeys (FIG. 13C and FIG. 20). Across multiple days of testing (n=2 sessions), when the stimulation was turned ON at 55 Hz, TBI01 was able to reduce the amount of grip force deviation compared to when the stimulation was OFF. DBS-ON performances were smoother and more accurate when compared to DBS-OFF (FIG. 26E, RMSE, Day 1: 0.05 (DBS ON) vs 0.07 (DBS OFF), Day 2: 0.05 (DBS ON) vs 0.14 (DBS OFF)). Importantly, the tremor was consistent with and without stimulation at 55 Hz demonstrating that the improved voluntary control was not caused by a reduction in the level of tremor (FIG. 26F). Additionally, these effects persisted regardless of the testing order of DBS-ON and DBS-OFF, and they were therefore not induced by learning effects.

These results suggest that motor thalamus DBS at optimal stimulation frequencies (50-100 Hz) can be applied to improve volitional fine motor control in patients with chronic lesions of the CST.

### Example 15

DBS Potentiates Kinematics and MEP in the Face during Volitional Movements

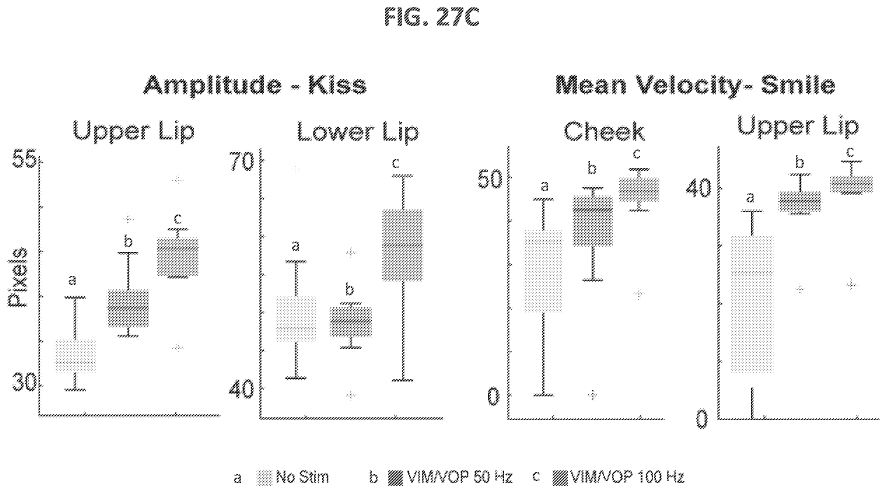

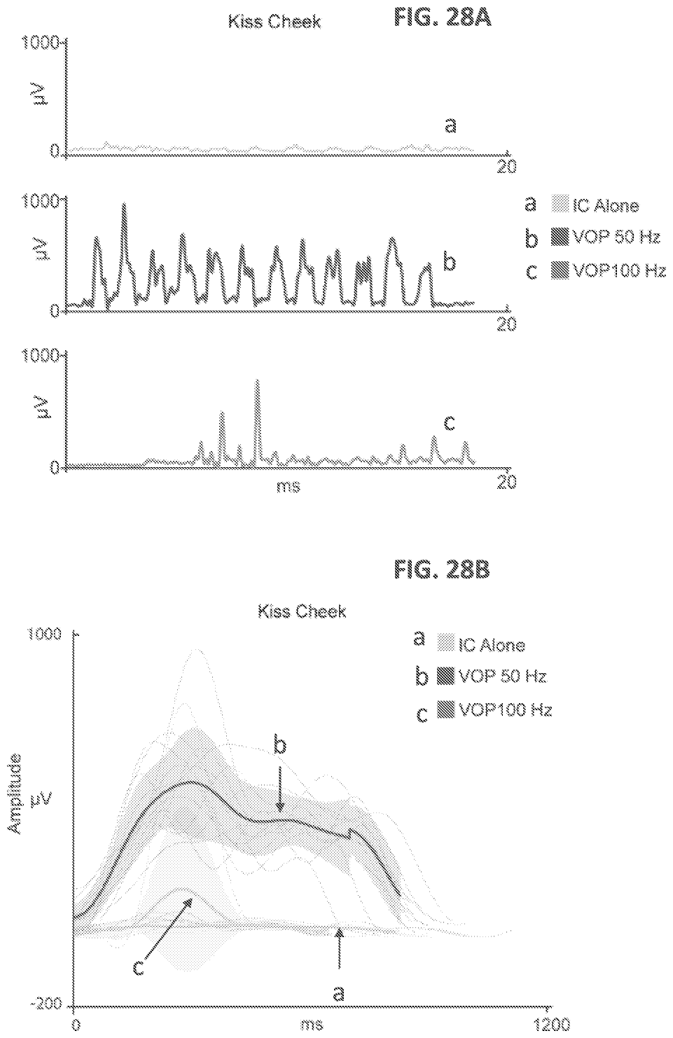

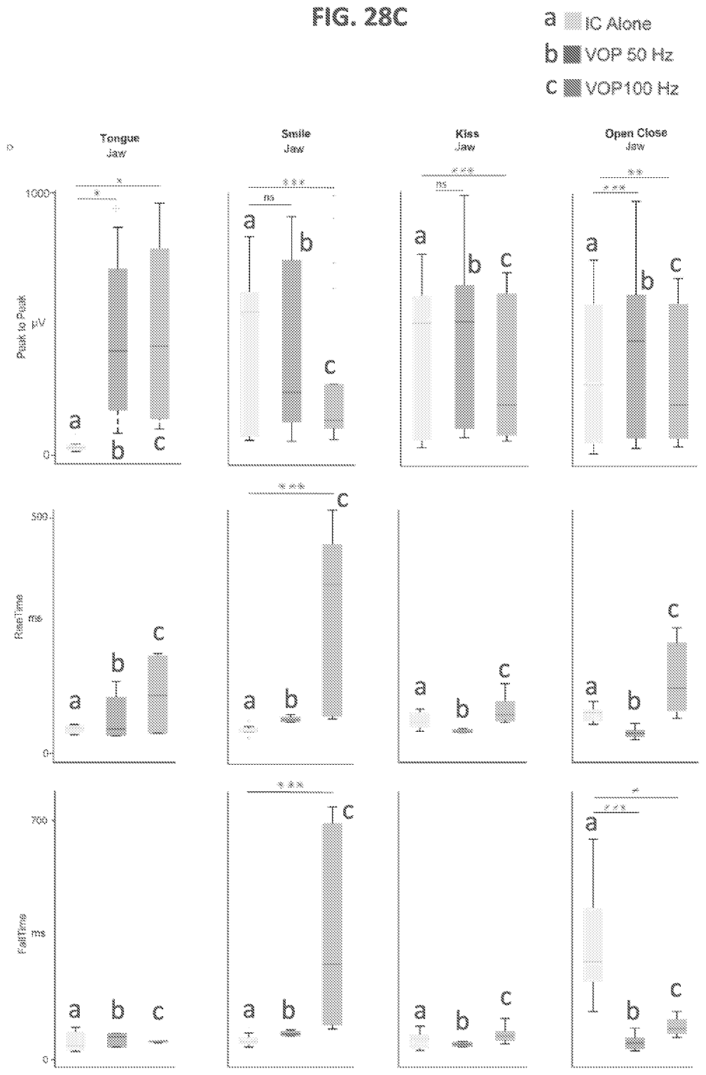

Human participants with a history of seizures were recruited and implanted with 10-15 SEEG electrodes in the thalamus and frontal cortical area for seizure monitoring. Participants had no history of stroke or observed impairments of speech. During the epilepsy monitoring data was collected from 60 minute sessions of VOP stimulation paradigms. Surface EMGs were used to monitor the contralateral muscles: geniohyoid (tongue), masseter (jaw), and zygomaticus (cheek). Subjects performed speech-therapy exercises that involve moving from an instructed facial expression to a neutral expression as many times as possible in a 20-second window. These tasks were repeated without VOP stimulation and with VOP stimulation at 50 Hz stimulation and 100 Hz. Movements of the mouth were tracked using DeepLabCut on the video recordings (FIG. 27A). Kinematic trace features including amplitude, task duration width, rise velocity, and fall velocity were extracted directionally for each of the face markers (e.g. chin, corner of mouth, bottom lip, top lip, tongue). Dimensionality reduction using PCA was performed to visualize separation between the stimulation conditions (FIG. 27B). Individual kinematic features were assessed across stimulation conditions, and significant differences were identified between the stim and no stim conditions (FIG. 27C). EMG data were assessed for different stimulation conditions (FIG. 28A). There was clear oscillation in the muscle activity as the participants performed the task, with increased amplitudes of the 50 Hz and 100 Hz EMGs. The EMG traces were cut by visual inspection to identify only the traces of the instructed facial expression. These traces were then averaged to assess the differences in condition traces (FIG. 28B). Muscle trace features were extracted and compared for significant potentiation between the conditions (FIG. 28C).

It will be apparent that the precise details of the methods or compositions described may be varied or modified without departing from the spirit of the described examples. We claim all such modifications and variations that fall within the scope and spirit of the claims below

1. A method for treating a human subject having a motor disorder, the method comprising:
applying an electrical stimulus to neurons in the thalamus, wherein the neurons comprise axons projecting to premotor or motor cortex, and wherein the electrical stimulus is applied with one or more electrodes controlled by a neurostimulator,
wherein the electrical stimulus improves at least one motor output associated with the motor disorder.

2. The method according to claim 1, wherein the electrical stimulus comprises electrical pulses having an amplitude of less than about 10 mA, pulse widths between about 80 μs and about 2 ms, and/or a pulse frequency between about 20 Hz and about 100 Hz.

3. The method according to claim 2, wherein the electrical stimulus comprises electrical pulses having an amplitude of between about 1.5 and about 3.5 mA, pulse widths between about 100 μs and about 200 μs, and/or a pulse frequency between about 40 Hz and about 80 Hz.

4. The method according to claim 1, wherein the one or more electrodes are configured to selectively apply the electrical stimulus to axons of the neurons in at least one motor and/or sensory area(s) of the ventral thalamus.

5. The method according to claim 4, wherein the at least one motor and/or sensory area(s) of the ventral thalamus is selected from the group consisting of the ventral anterior nucleus (VA), the ventral oral posterior nucleus (VOP), the ventral intermediate nucleus (VIM), the ventral oral anterior nucleus (VOA), the ventral caudal nucleus (VC), a lateral area of the VOP and/or VIM, a lateral area of the VOP and/or VIM associated with arm movements, and a medial area of the VOP associated with face movements.

6. The method according to claim 5, wherein the at least one motor and/or sensory area(s) of the ventral thalamus comprise the VIM and the VOP;
the electrical stimulus comprises electrical pulses having a pulse frequency between about 50 Hz and about 80 Hz; and
the motor disorder of the subject comprises a motor impairment of the arms, fingers, or hands causing a loss of muscle strength, partial paralysis, paresis, loss of dexterity, reduced movement, uncontrollable muscle tone, or essential tremor.

7. The method according to claim 5, wherein the at least one motor and/or sensory area(s) of the ventral thalamus comprises the VOA;
the electrical stimulus comprises electrical pulses having a pulse frequency between about 50 Hz and about 100 Hz; and
the motor disorder of the subject is a speech disorder resulting in at least one speech motor impairment.

8. The method according to claim 1, wherein the neurostimulator satisfies at least one condition selected from the group of conditions consisting of:
the neurostimulator is externalized;
the neurostimulator is implanted in the subject;
the neurostimulator is implanted in the chest of the subject;
the neurostimulator is implanted in the belly of the subject;
the neurostimulator is fully implanted in the brain of the subject;
the neurostimulator is a daily assistive device or is a component of a daily assistive device;
the neurostimulator is a current or voltage-controlled stimulator;
the neurostimulator comprises at least one multiple contact lead configured to produce orientation selective axonal stimulation; and
the neurostimulator controls the applying of an electrical stimulus comprising at least one of:
charge balanced pulses,
a continuous electrical stimulus, and
a closed-loop electrical stimulus.

9. The method according to claim 1,
wherein the motor disorder comprises a motor impairment causing a loss of muscle strength, a speech deficit, oral apraxia, dysarthria, partial paralysis, paresis, loss of dexterity, reduced hand movement, reduced finger movement, discoordination of the oral/deglutition function, dysphagia, uncontrollable muscle tone, essential tremor, and/or dystonia, and
wherein the motor disorder results from at least one of ischemic brain injury, hemorrhagic brain injury, traumatic brain injury, brain injury caused by stroke, a neurodegenerative disorder, Parkinson's disease, brain tumor, muscular dystrophy, myasthenia gravis, cerebral palsy, multiple sclerosis, amyotrophic lateral sclerosis (ALS), and transection of eloquent motor-related and/or speech-related gray or white matter.

10. The method according to claim 9, wherein the motor disorder results from ALS.

11. The method according to claim 9, wherein the motor disorder results from traumatic brain injury.

12. The method according to claim 1, wherein the at least one motor output associated with the motor disorder is a voluntary motor output comprising at least one of:
a motor output associated with hand movement;
a motor output associated with finger movement;
a motor output associated with arm paresis;
a motor output associated with hand paresis;
the amplitude of an arm movement of the subject;
the fine motor control of an arm movement of the subject; and
the amplitude of grip strength of the subject.

13. The method according to claim 1, wherein the motor disorder of the subject is a speech disorder resulting in at least one speech motor impairment.

14. The method according to claim 13, wherein the at least one speech motor impairment comprises dysarthria, speech arrest, oral apraxia, and/or dysphagia.

15. The method according to claim 14, wherein the at least one speech motor impairment comprises dysarthria or oral apraxia.

16. The method according to claim 13, wherein the at least one motor output associated with the speech disorder is a voluntary motor output comprising at least one of:
a motor output associated with a lost speech deficit;
a motor output associated with an impaired speech deficit;
reduced amplitude and/or fine motor control of facial movement;
reduced amplitude and/or fine motor control of neck movement;
reduced amplitude and/or fine motor control of tongue movement;
reduced speech production;
reduced syllabic articulation;
reduced syllabic volume;
increased delay between syllables;
speech arrest;
involuntary repetition of syllables;
prolongation of sounds;
interruptions in speech;
indistinct pronunciation of syllables;
mumbling of syllables;
a motor output associated with dysphasia;
reduced swallowing; and
a motor output associated with painful swallowing.

17. The method according to claim 1, wherein the neurostimulator controls the application of an electrical stimulus in a phasic manner, and wherein the stimulation parameters change over the course of a voluntary movement or while the subject is speaking.

18. The method according to claim 1, wherein the electrical stimulus is applied at or below a motor threshold of the human subject, and/or at or below the perceptual threshold of the human subject.
